(19) United States
(12) Patent Application Publication
(10) Pub. No.: US 2025/0145726 A1
(43) Pub. Date: May 8, 2025

(54) INNATE IMMUNE CHECKPOINT MODULATORS

(71) Applicant: Whitehead Institute for Biomedical Research, Cambridge, MA (US)

(72) Inventors: Kipp Weiskopf, Wellesley, MA (US); Jonathan S. Weissman, Cambridge, MA (US); Dian Yang, Cambridge, MA (US)

(21) Appl. No.: 18/837,529

(22) PCT Filed: Feb. 10, 2023
(86) PCT No.: PCT/US2023/012857

§ 371 Date: Aug. 9, 2024

Related U.S. Application Data
(60) Provisional application No. 63/411,612, filed on Sep. 29, 2022.
(60) Provisional application No. 63/309,448, filed on Feb. 11, 2022.

(51) Int. Cl. C07K 16/28; A61P 35/00; C12N 5/0786; C12N 9/16; G01N 33/50

(52) U.S. Cl. CPC C07K 16/2878; A61P 35/00; C07K 16/2896; C12N 5/0645; C12N 9/16; G01N 33/502; G01N 33/5055; G01N 2500/10

(57) ABSTRACT

Provided herein are compositions and methods of modulating myeloid cell-mediated killing of cancer cells and modulating the activity of myeloid cell immune checkpoint inhibitors. Also provided herein are methods of screening for modulators of myeloid cell-mediated killing of cancer cells and modulators of myeloid cell immune checkpoint inhibitors.

### RELATED APPLICATION(S)

This application claims the benefit of U.S. Provisional Application No. 63/309,448 filed on Feb. 11, 2022, and U.S. Provisional Application No. 63/411,612, filed on Sep. 29, 2022. The entire teachings of the above applications are incorporated herein by reference.

### BACKGROUND OF THE INVENTION

Immunotherapy has revolutionized cancer treatment with most efforts focused on enhancing T cell responses using immune checkpoint inhibitors. Yet, despite its success, only a small subset of patients actually benefits from immunotherapy, and it can cause severe side effects. The lack of progress in developing next-generation agents that target T cells has renewed interest in identifying new targets.

As a major component of the innate immune system, myeloid cells, especially macrophages, are attractive targets for several reasons. 1) Myeloid cells can kill tumor cells by multiple mechanisms—phagocytosis, trogocytosis and secretion of cytotoxic factors; 2) in contrast to T cells, they are highly infiltrative, sometimes accounting for up to 50% of the cells within solid tumors; and 3) myeloid cells can activate the adaptive immune system via antigen presentation. Given the crucial role of myeloid cells in antitumor immunity, an improved understanding of the mechanisms that regulate tumor-myeloid cell interactions and the identification of novel myeloid-directed therapies could identify new opportunities to enable tumor destruction.

Although there is no FDA-approved therapy that targets myeloid cells in cancer yet, many therapeutic strategies are being pursued. The main approaches include blocking myeloid recruitment and survival, activating myeloid-mediated killing, and reprogramming myeloid activation states. Among those, myeloid immune checkpoints (myeICs) have been demonstrated as essential mechanisms for tumor immune evasion through inhibition of phagocytosis and suppression of innate immune signaling, making them appealing targets for immunotherapy. Targeting the CD47-SIRPa axis, the most well-characterized myeIC, has shown remarkable efficacy in preclinical studies and early clinical trials of multiple cancer types. The anti-tumor effects of CD47 blockade are through macrophage-mediated effects, and through cross-priming of T cells by dendritic cells (DCs) and macrophages for tumor elimination. However, CD47 blockade has limited efficacy in some solid tumors and on-target toxicity to red blood cells. Currently, only a limited number of myeICs have been reported, including CD24, PD-L1, MHC-I and APMAP. Importantly, blocking these myeICs individually could inhibit tumor growth and synergize with CD47-blocking therapy to overcome resistance to CD47 blockade, highlighting the importance of identifying new myeICs.

Despite these encouraging results, myeICs remain poorly characterized for three major reasons: 1) focus has been placed primarily on adaptive immune checkpoints; 2) there are few unbiased and systematic studies of myeICs in the context of tumor-immune evasion 42; 3) current research has focused on immune cells with less emphasis on the evolution of cancer cells. Thus, the identification of myeICs in cancer using unbiased approaches will greatly expand our understanding of cancer-myeloid cell interactions and might identify new therapeutic strategies.

### SUMMARY OF THE INVENTION

Some aspects of the present disclosure are directed to a method of screening for a modulator of myeloid cell-mediated killing of cancer cells, comprising a) providing a population of cancer cells expressing a targetable endonuclease and an sgRNA library targeting genes, b) contacting the cancer cells with myeloid cells capable of having an anticancer response, c) coculturing the cancer cells and the myeloid cells, and d) measuring the relative abundance of each sgRNA of the sgRNA library in the cocultured cancer cells as compared to the abundance of each gRNA in control cancer cells not contacted with the myeloid cells, wherein the differential relative abundance of an sgRNA as compared to the control indicates that the gene targeted by the sgRNA is a candidate modulator of myeloid cell-mediated killing of cancer cells.

In some embodiments, the population of cancer cells of step a) has been transduced with a pool of sgRNA targeting every gene expressed on the cell surface in the genome of the cancer cell at a multiplicity of infection of about 0.2 to 0.4. In some embodiments, the population of cancer cells of step a) has been transduced with a pool of sgRNA targeting every gene in the genome of the cancer cell at a multiplicity of infection of about 0.2 to 0.4.

In some embodiments, least two sgRNA target each gene of the targeted genes.

In some embodiments, the cancer cells are a cancer cell line (e.g., a lung cancer cell line including, but not limited to, PC9 and NCI-H358). In some embodiments, the myeloid cells are macrophages (e.g., macrophages that have been produced by ex vivo differentiation of monocytes), such as human macrophages. In some embodiments, the population of cancer cells expresses the targetable endonuclease. In some embodiments, the cancer cells and myeloid cells are cocultured for 1 day or more in step c).

In some embodiments, an increased abundance of sgRNA targeting a gene as compared to the control indicates that the product of the gene enhances myeloid cell-mediated killing of the cancer cells. In some embodiments, a decreased abundance of sgRNA targeting a gene as compared to the control indicates that the product of the gene inhibits myeloid cell-mediated killing of the cancer cells.

In some alternate aspects, the cancer cell is replaced with a target cell. For example, it may be desirable to modulate myeloid cell-mediated killing of allogenic cells, IPSC-derived cells, damaged cells, or cells of particular cell types such as neuronal cells, pre-cancerous cells, liver cells, etc. Thus, some alternate aspects of the present disclosure are directed to a method of screening for a modulator of myeloid cell-mediated killing of target cells, comprising a) providing a population of target cells expressing a targetable endonuclease and an sgRNA library targeting genes or an interfering RNA library, b) contacting the target cells with myeloid cells capable of having an anti-target cell response, c) coculturing the target cells and the myeloid cells, and d) measuring the relative abundance of each sgRNA of the sgRNA library or interfering RNA of the interfering RNA library in the cocultured target cells as compared to the abundance of each in control target cells not contacted with the myeloid cells, wherein the differential relative abundance of an sgRNA or interfering RNA as compared to the control indicates that the gene targeted by the sgRNA or interfering RNA is a candidate modulator of myeloid cell-mediated killing of the target cells. The target cell is not limited and may be any suitable cell. In some embodiments, the target cell is muscle cell, brain cell, neuronal cell, liver cell, kidney cell, digestive tract cell, bone cell, cartilage cell, heart cell, lung cell, infected cell, fetal cell, endocrine cell, lymphatic cell, or epidermal cell.

Some aspects of the present disclosure are directed to a method of screening for a modulator of myeloid cell-mediated killing of cancer cells, comprising a) providing a population of cancer cells expressing an RNAi library, b) contacting the cancer cells with myeloid cells capable of having an anticancer response, c) coculturing the cancer cells and the myeloid cells, and d) measuring the relative abundance of each RNAi agent of the RNAi library in the cocultured cancer cells as compared to the abundance of each RNAi agent in control cancer cells not contacted with the myeloid cells, wherein the differential relative abundance of an RNAi agent as compared to the control indicates that the gene targeted by the RNAi is a candidate modulator of myeloid cell-mediated killing of cancer cells.

Some aspects of the present disclosure are directed to a method of screening for a modulator of myeloid cell-mediated killing of cancer cells, comprising a) providing a population of myeloid cells capable of having an anticancer response and expressing a targetable endonuclease and an sgRNA library targeting genes, b) contacting cancer cells with the myeloid cells, c) coculturing the cancer cells and the myeloid cells, and d) identifying targeted genes in the myeloid cells that enhance or inhibit myeloid cell-mediated killing of cancer cells.

Some aspects of the present disclosure are directed to a method of screening for a modulator of myeloid cell-mediated killing of cancer cells, comprising a) providing a population of myeloid cells capable of having an anticancer response and expressing an RNAi library, b) contacting cancer cells with the myeloid cells, c) coculturing the cancer cells and the myeloid cells, and d) identifying targeted genes in the myeloid cells that enhance or inhibit myeloid cell-mediated killing of cancer cells.

Some aspects of the present disclosure are directed to a method of screening for a modulator of myeloid cell-mediated killing of cancer cells in the presence of a myeloid cell checkpoint inhibitor, comprising a) providing a population of cancer cells expressing a targetable endonuclease and an sgRNA library targeting genes, b) contacting the cancer cells with myeloid cells capable of having an anticancer response and the myeloid cell checkpoint inhibitor, c) coculturing the cancer cells and myeloid cells with the myeloid cell checkpoint inhibitor, and d) measuring the relative abundance of each sgRNA of the sgRNA library in the cocultured cancer cells as compared to the abundance of each gRNA in control cancer cells cocultured with myeloid cells but not the myeloid cell checkpoint inhibitor, wherein the differential relative abundance of an sgRNA as compared to the control indicates that the gene targeted by the sgRNA is a candidate modulator of myeloid cell-mediated killing of cancer cells in the presence of the myeloid cell checkpoint inhibitor.

In some embodiments, the population of cancer cells of step a) has been transduced with a pool of sgRNA targeting every gene expressed on the cell surface in the genome of the cancer cell at a multiplicity of infection of about 0.2 to 0.4. In some embodiments, the population of cancer cells of step a) has been transduced with a pool of sgRNA targeting every gene in the genome of the cancer cell at a multiplicity of infection of about 0.2 to 0.4.

In some embodiments, at least two sgRNA target each gene of the targeted genes.

In some embodiments, the cancer cells are a cancer cell line, such as a lung cancer cell line (e.g., Pc9 or NCI-H358). In some embodiments, the myeloid cells are macrophages, e.g., human macrophages. In some embodiments, the population of cancer cells expresses the targetable endonuclease.

In some embodiments, the myeloid cell checkpoint inhibitor is a CD24 antibody, a CD47 antagonist, a CD40 agonist, or a PD-L1 antagonist.

In some embodiments, the cancer cells and myeloid cells are cocultured with the myeloid cell checkpoint inhibitor for 1, 2, 3, 4, or 5 days or more in step c). In some embodiments, an increased abundance of sgRNA targeting a gene as compared to the control indicates that the product of the gene enhances myeloid cell-mediated killing of cancer cells in the presence of a myeloid cell checkpoint inhibitor. In some embodiments, a decreased abundance of sgRNA targeting a gene as compared to the control indicates that the product of the gene inhibits myeloid cell-mediated killing of cancer cells in the presence of a myeloid cell checkpoint inhibitor.

Some aspects of the present disclosure are directed to a method of screening for a modulator of myeloid cell-mediated killing of cancer cells in the presence of a myeloid cell checkpoint inhibitor, comprising a) providing a population of cancer cells expressing a RNAi library, b) contacting the cancer cells with myeloid cells capable of having an anticancer response and the myeloid cell checkpoint inhibitor, c) coculturing the cancer cells and myeloid cells with the myeloid cell checkpoint inhibitor, and d) measuring the relative abundance of each RNAi agent of the RNAi library in the cocultured cancer cells as compared to the abundance of each RNAi agent in control cancer cells cocultured with myeloid cells but not the myeloid cell checkpoint inhibitor, wherein the differential relative abundance of an RNAi agent as compared to the control indicates that the gene targeted by the RNAi agent is a candidate modulator of myeloid cell-mediated killing of cancer cells in the presence of the myeloid cell checkpoint inhibitor.

Some aspects of the present disclosure are directed to a method of screening for a modulator of myeloid cell-mediated-killing of cancer cells in the presence of a myeloid cell checkpoint inhibitor, comprising a) providing a population of myeloid cells capable of having an anticancer response and expressing a targetable endonuclease and an sgRNA library targeting genes, b) contacting cancer cells with the myeloid cells, c) coculturing the cancer cells and myeloid cells with the myeloid cell checkpoint inhibitor, and d) identifying targeted genes in the myeloid cells that enhance or inhibit myeloid cell-mediated killing of cancer cells.

Some aspects of the present disclosure are directed to a method of screening for a modulator of myeloid cell-mediated killing of cancer cells in the presence of a myeloid cell checkpoint inhibitor, comprising a) providing a population of myeloid cells capable of having an anticancer response and expressing an RNAi library, b) contacting cancer cells with the myeloid cells, c) coculturing the cancer cells and myeloid cells with the myeloid cell checkpoint inhibitor, and d) identifying targeted genes in the myeloid cells that enhance or inhibit myeloid cell-mediated killing of cancer cells.

Some aspects of the present disclosure are directed to a method of treating cancer in a subject comprising administering to the subject an agent that modulates the level or activity of a cancer cell gene that modulates macrophage-mediated cancer cell killing (MMCCK).

In some embodiments, the agent decreases the level or activity of the cancer cell gene, and the cancer cell gene inhibits MMCCK (e.g., Don't Eat Me (DEM) signal).

In some embodiments, the cancer cell gene is selected from Met, Cd47, Igf1r, Arf1, Notch2, Afdn, Art1, Msn, Slc16a1, Gnai2, Sdc1, Cd4, Cd163, Cftr, Cd8a, Jam2, Icos, Nrg1, Ide, I112rb2, Has2, Gpc1, Insr, Epha2, Jmjd6, and Lrrc4.

In some embodiments, the agent is an antibody to a cell surface receptor or functional fragment or derivative of the antibody.

In some embodiments, the agent increases the level or activity of the cancer cell gene and the cancer cell gene enhances MMCCK (e.g., Eat Me (EM) signal).

In some embodiments, the cancer cell gene is selected from Acvr1b, Acvr2a, Adam9, Adcy1, Atp6ap2, Bmpr2, C5ar2, Cd320, Cd7, Cdc20, Cdh1, Cdh11, Epha4, Fxyd6, Gjb1, Hras, Ifn1r1, I110ra, I113ra1, I121r, Itgav, Itgb1, Itgb3, Lamc2, Lrfn3, Plxnb2, Po1r1c, Psen1, Ptdss1, Pth2r, Ror2, Rtn4r12, Sor11, St14, Stx4a, Tfrc, T1r6, and Tspan1.

Some aspects of the present disclosure are directed to a method of treating cancer in a subject comprising administering to the subject a myeloid cell checkpoint inhibitor and an agent that modulates the level or activity of a cancer cell gene that modulates myeloid cell-mediated killing of cancer cells in the presence of the myeloid cell checkpoint inhibitor.

In some embodiments, the agent decreases the level or activity of the cancer cell gene and the cancer cell gene inhibits myeloid cell-mediated killing of cancer cells in the presence of the myeloid cell checkpoint inhibitor.

In some embodiments, the myeloid cell checkpoint inhibitor is an anti-CD24 antibody that binds, blocks or opsonizes, and the cancer cell gene is selected from Cd24a, Acvr1b, Acvr2a, Ncstn, Psen1, Itgb1, Tgfbr1, Epha2, Cd320, F2r, Nt5e, Sdc1, Efnb3, Pdcd11g2, Hjv, Rnf43, Adam23, Havcr2, Lag3, Erbb2, Art1, Insr, T1r6, Cdh11, T1r2, I117rc, Adora2b, Tfrc, Dnajb11, Ramp3, Igf1r, Arf1, Acvr1, Afdn, Tnfsf13, Ld1r, Atp5b, Atp6ap2, Stx4a, Cdh1, and Cd47. In some embodiments, the cancer cell gene inhibits the activity of the anti-CD24 blocking antibody, and the agent inhibits a level or activity of the cancer cell gene. In some embodiments, the cancer cell gene that inhibits the activity of the anti-CD24 blocking antibody is selected from Cd24a, Acvr1b, Acvr2a, Ncstn, Psen1, Itgb1, Tgfbr1, Epha2, Cd320, F2r, Nt5e, and Sdc1. In some embodiments, the cancer cell gene increases or synergizes with the activity of the anti-CD24 blocking antibody, and the agent increases a level or activity of the cancer cell gene. In some embodiments, the cancer cell gene that increases or synergizes with the activity of the anti-CD24 blocking antibody is selected from Efnb3, Pdcd11g2, Hjv, Rnf43, Adam23, Havcr2, Lag3, Erbb2, Art1, Insr, T1r6, Cdh11, T1r2, I117rc, Adora2b, Tfrc, Dnajb11, Ramp3, Igf1r, Arf1, Acvr1, Afdn, Tnfsf13, Ld1r, Atp5b, Atp6ap2, Stx4a, Cdh1, and Cd47.

In some embodiments, the myeloid cell checkpoint inhibitor is an anti-CD47 blocking antibody, and the cancer cell gene is selected from Rpsa, Acvr1b, Acvr2a, Ncstn, Alcam, Tmem222, Psen1, Igsf11, Fzd5, Plxnb2, Cadm1, Lrp5, Itgb3, Cd99, Retn, Egfr, Atp6ap2, K1rb1a, Adam10, Lamp1, C5ar1, Sstr5, Lrfn3, Sema4b, Igf1r, Ld1r, Fam3c, Met, Erbb2, Cdh11, I121r, I117rc, Adgrb2, Atp5b, Arf1, Copa, Acvr1, and Stx4a. In some embodiments, the cancer cell gene inhibits the activity of the anti-CD47 blocking antibody, and the agent inhibits a level or activity of the cancer cell gene. In some embodiments, the cancer cell gene that inhibits the activity of the anti-CD47 blocking antibody is selected from Rpsa, Acvr1b, Acvr2a, Ncstn, Alcam, Tmem222, Psen1, Igsf11, Fzd5, Plxnb2, Cadm1, and Lrp5. In some embodiments, the cancer cell gene increases or synergizes with the activity of the anti-CD47 blocking antibody, and the agent increases a level or activity of the cancer cell gene. In some embodiments, the cancer cell gene that increases or synergizes with the activity of the anti-CD47 blocking antibody is selected from Itgb3, Cd99, Retn, Egfr, Atp6ap2, K1rb1a, Adam10, Lamp1, C5ar1, Sstr5, Lrfn3, Sema4b, Igf1r, Ld1r, Fam3c, Met, Erbb2, Cdh11, I121r, I117rc, Adgrb2, Atp5b, Arf1, Copa, Acvr1, and Stx4a.

In some embodiments, the myeloid cell checkpoint inhibitor is an anti-CD40 agonizing antibody, and the cancer cell gene is selected from Rpsa, Cdc20, Mfrp, Igf1r1, I118r1, I127ra, Ephb2, Adam19, Pdcd1, and Copa. In some embodiments, the cancer cell gene inhibits the activity of the anti-CD40 agonizing antibody, and the agent inhibits a level or activity of the cancer cell gene. In some embodiments, the cancer cell gene that inhibits the activity of the anti-CD40 agonizing antibody is selected from Rpsa, Cdc20, Mfrp, and Igf1r1. In some embodiments, the cancer cell gene increases or synergizes with the activity of the anti-CD40 agonizing antibody, and the agent increases a level or activity of the cancer cell gene. In some embodiments, the cancer cell gene that increases or synergizes with the activity of the anti-CD40 agonizing antibody is selected from I118r1, I127ra, Ephb2, Adam19, Pdcd1, and Copa.

In some embodiments, the myeloid cell checkpoint inhibitor is an anti-PD-L1 (sometimes referred to as anti-PDL1) blocking antibody, and the cancer cell gene is selected from Nectin2, Ltk, Erbb3, Mp1, Ptprd, Mrc1, Tspan1, Egfr, I117rc, Sdc2, Stx3, Ntrk1, Sstr5, Cdh11, and Copa. In some embodiments, the cancer cell gene inhibits the activity of the anti-PD-L1 blocking antibody, and the agent inhibits a level or activity of the cancer cell gene. In some embodiments, the cancer cell gene that inhibits the activity of the anti-PD-L1 blocking antibody is selected from Nectin2 and Ltk. In some embodiments, the cancer cell gene increases or synergizes with the activity of the anti-PD-L1 blocking antibody, and the agent increases a level or activity of the cancer cell gene. In some embodiments, the cancer cell gene that increases or synergizes with the activity of the anti-PD-L1 blocking antibody is selected from Erbb3, Mp1, Ptprd, Mrc1, Tspan1, Egfr, I117rc, Sdc2, Stx3, Ntrk1, Sstr5, Cdh11, and Copa.

In some embodiments, the agent increases the level or activity of the cancer cell gene, and the cancer cell gene enhances myeloid cell-mediated killing of cancer cells in the presence of the myeloid cell checkpoint inhibitor.

In some embodiments, the myeloid cell checkpoint inhibitor is an anti-CD24 blocking antibody, and the cancer cell gene is selected from Efnb3, Pdcd11g2, Hjv, Rnf43, Adam23, Havcr2, Lag3, Erbb2, Art1, Insr, T1r6, Cdh11, T1r2, I117rc, Adora2b, Tfrc, Dnajb11, Ramp3, Igf1r, Arf1, Acvr1, Afdn, Tnfsf13, Ld1r, Atp5b, Atp6ap2, Stx4a, Cdh1, and Cd47.

In some embodiments, the myeloid cell checkpoint inhibitor is an anti-CD47 blocking antibody, and the cancer cell gene is selected from Itgb3, Cd99, Retn, Egfr, Atp6ap2, K1rb1a, Adam10, Lamp1, C5ar1, Sstr5, Lrfn3, Sema4b, Igf1r, Ld1r, Fam3c, Met, Erbb2, Cdh11, I121r, I117rc, Adgrb2, Atp5b, Arf1, Copa, Acvr1, and Stx4a.

In some embodiments, the myeloid cell checkpoint inhibitor is an anti-CD40 agonizing antibody, and the cancer cell gene is selected from I118r1, I127ra, Ephb2, Adam19, Pdcd1, and Copa.

In some embodiments, the myeloid cell checkpoint inhibitor is an anti-PD-L1 blocking antibody, and the cancer cell gene is selected from Erbb3, Mp1, Ptprd, Mrc1, Tspan1, Egfr, I117rc, Sdc2, Stx3, Ntrk1, Sstr5, Cdh11, and Copa.

In some embodiments, the subject is a mouse, a human, or other mammal.

Some aspects of the present disclosure are directed to a pharmaceutical composition comprising a pharmaceutically acceptable excipient and an antibody or functional fragment or derivative thereof specifically binding to Ermp1, Cflar, Slc35a1, Chst2, Copx, Map3k7, Efr3a, Dpm1, Dpm2, Dpm3, or PigP.

Some aspects of the present disclosure are directed to a pharmaceutical composition comprising a pharmaceutically acceptable excipient and an agent that increases the level or activity of Ptdss1, Mtf1, Zbtb14, or Pomp.

All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

The above discussed, and many other features and attendant advantages of the present inventions will become better understood by reference to the following detailed description of the invention.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a graph comparing the representation of sgRNAs in cancer cells (using cell line KP-238N1) when co-culturing with primary macrophage vs. cancer cells cultured alone. Gene hits in the upper left quadrant (having negative enrichment) indicate that these genes provide “Don't Eat Me” signals. Gene hits in the upper right quadrant (having positive enrichment) indicate that these genes provide “Eat Me” signals.

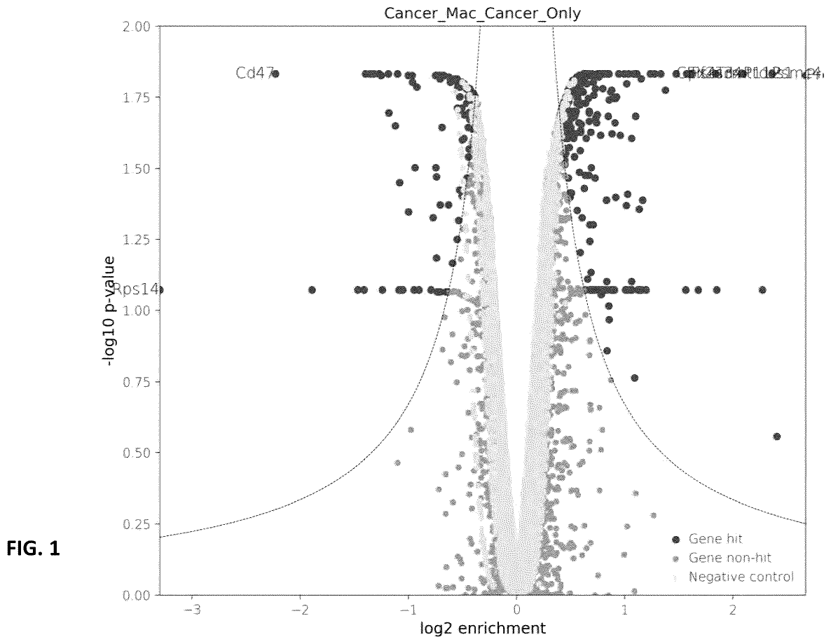

FIG. 2 is a graph comparing the representation of sgRNA in cancer cells (cell line KP-368T1) when co-culturing with primary macrophage vs. cancer cells cultured alone. Gene hits in the upper left quadrant (having negative enrichment) indicate that these genes provide “Don't Eat Me” signals. Gene hits in the upper right quadrant (having positive enrichment) indicate that these genes provide “Eat Me” signals.

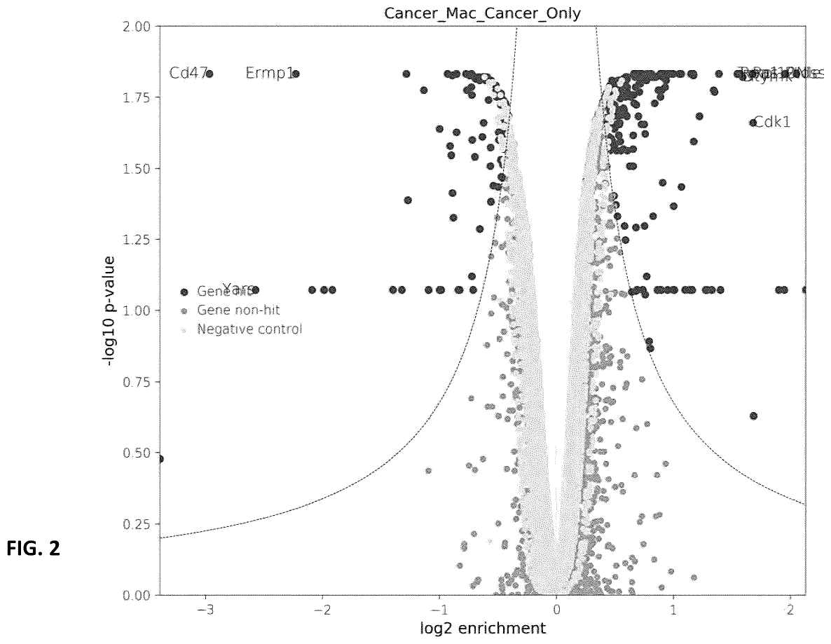

FIG. 3 is a graph comparing the representation of sgRNA in cancer cells (cell line KP-238N1) when co-culturing with primary macrophage with vs. without anti-CD24 binding antibody treatment. Gene hits in the upper left quadrant (having negative enrichment) indicate that these genes enhance the therapeutic effect of Anti-CD24 treatment. Gene hits in the upper right quadrant (having positive enrichment) indicate that these genes decrease the therapeutic effect of Anti-CD24 treatment.

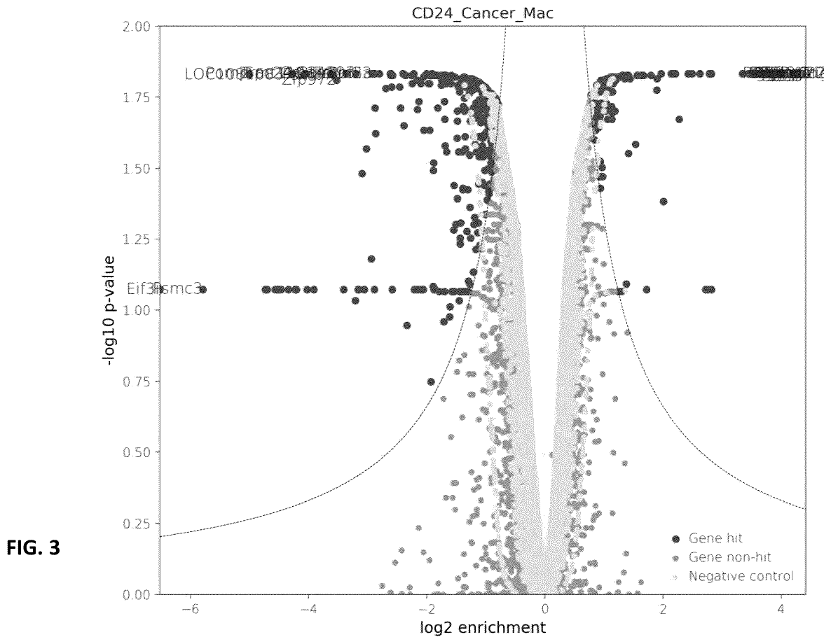

FIG. 4 is a graph comparing the representation of sgRNA in cancer cells (cell line KP-238N1) when co-culturing with primary macrophage with vs. without anti-CD47 blocking antibody treatment. Gene hits in the upper left quadrant (having negative enrichment) indicate that these genes enhance the therapeutic effect of anti-CD24 treatment. Gene hits in the upper right quadrant (having positive enrichment) indicate that these genes decrease the therapeutic effect of anti-CD24 treatment.

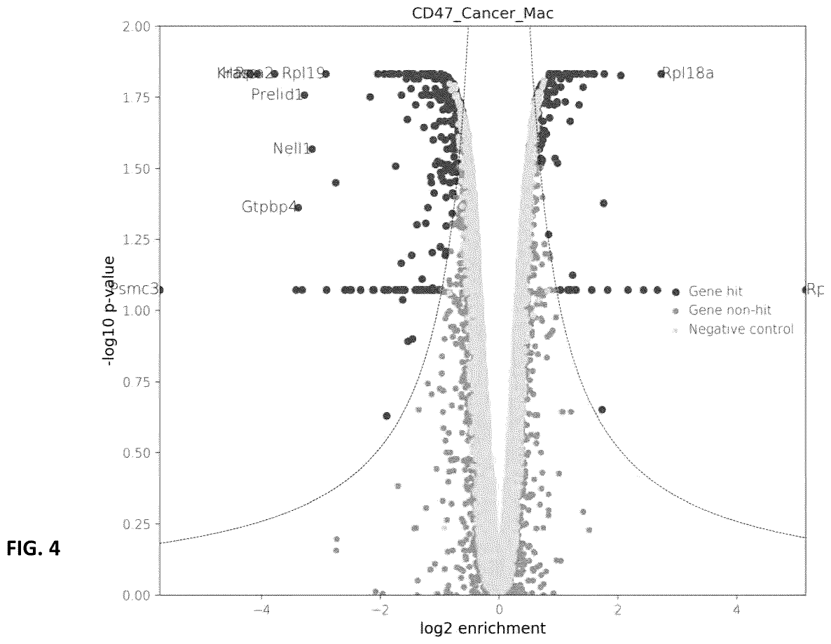

FIG. 5 is a schematic showing the details of genomic screens performed using primary human macrophages differentiated ex vivo from monocytes of human blood donors in the context of human lung cancer cell lines (PC9 and NCI-H358). Cancer cell lines were cultured either alone, in co-culture with human macrophages (monotherapy), or in co-culture with human macrophages and anti-CD47 agent (combination therapy).

FIG. 6 is a graph comparing the representation of sgRNAs in cancer cells co-cultured with human macrophages (left panel) or cancer cells co-cultured with human macrophages and anti-CD47 (right panel) as compared with cancer cells alone. Gene hits in the upper left quadrant (having negative enrichment) indicate that these genes provide “Eat Me” signals; knocking out expression of such genes reduces myeloid cell-mediated killing. Gene hits in the upper right quadrant (having positive enrichment) indicate that these genes provide “Don't Eat Me” signals; knocking out expression of such genes increases myeloid cell-mediated killing.

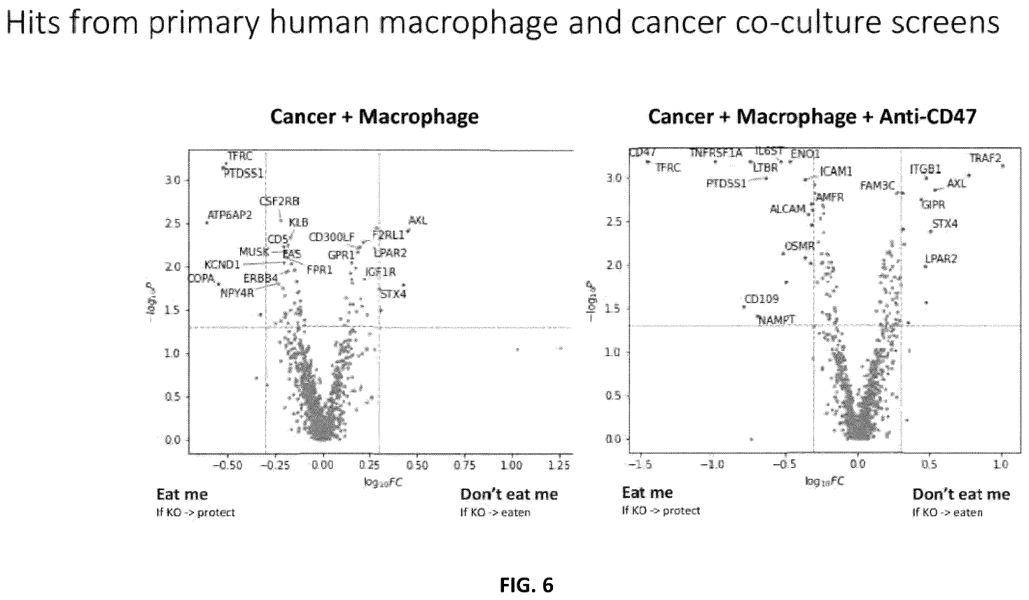

FIG. 7 is a graph showing that human macrophage polarization with IFN-γ (to M1-like) or IL-10 (to M2-like) does not substantially alter the screening results.

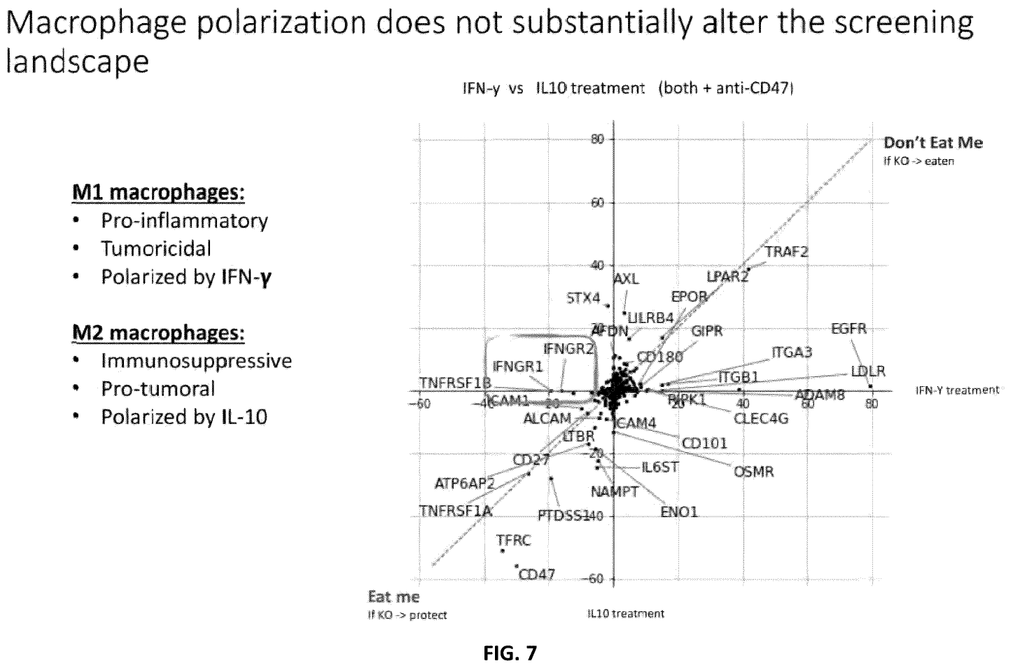

FIG. 8 is a graph demonstrating that consistent results are observed across multiple human macrophage donors.

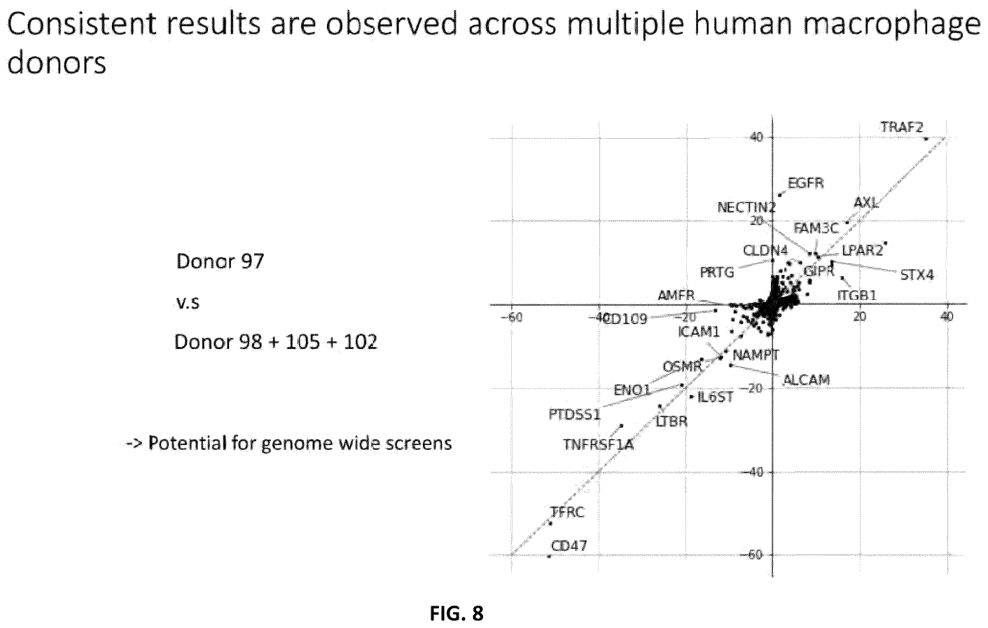

### DETAILED DESCRIPTION OF THE INVENTION

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, NJ, 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™ McKusick-Nathans Institute of Genetic Medicine, Johns Hopkins University (Baltimore, MD) and National Center for Biotechnology Information, National Library of Medicine (Bethesda, MD), as of May 1, 2010, ncbi.nlm.nih.gov/omim/and in Online Mendelian Inheritance in Animals (OMIA), a database of genes, inherited disorders and traits in animal species (other than human and mouse), at omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

The terms “decrease”, “reduced”, “reduction”, “decreases”, and “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase”, “increases”, “enhance” or “activate” are all used herein to generally mean an increase by a statically significant amount; for the avoidance of any doubt, the terms “increased”, “increase”, “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two-standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

### Screening for Modulators of Myeloid Cell-Mediated Killing

Some aspects of the present disclosure are directed to a method of screening for a modulator of myeloid cell-mediated killing of cancer cells, comprising a) providing a population of cancer cells expressing or otherwise comprising a targetable endonuclease and an sgRNA library targeting genes, b) contacting the cancer cells with myeloid cells capable of having an anticancer response, c) coculturing the cancer cells and the myeloid cells, and d) measuring the relative abundance of each sgRNA of the sgRNA library in the cocultured cancer cells as compared to the abundance of each gRNA in control cancer cells not contacted with the myeloid cells, wherein the differential relative abundance of an sgRNA as compared to the control indicates that the gene targeted by the sgRNA is a candidate modulator of myeloid cell-mediated killing of cancer cells. In some alternate embodiments, the population of myeloid cells instead of the population of cancer cell (or target cell as detailed below) is provided with a targetable endonuclease and an sgRNA library targeting genes (or RNA interference library as disclosed herein).

The type of cancer cell is not limited. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). The source organism for the cancer is not limited and may be any suitable animal. In some embodiments, the animal is a rat, mouse, dog, cat, horse, human, or non-human primate.

In some embodiments, the cancer is a cancer responsive to an immune checkpoint inhibitor (ICI). In some embodiments, the cancer is resistant or has developed resistance to an immune checkpoint inhibitor. In some embodiments, the ICI is a CD24 antibody, a CD47 antagonist, a CD40 agonist, a PD-1 antagonist, a CTLA-4 antagonist, a PD-L1 antagonist, a PD-L2 antagonist, or a LAG-3 agonist. In some embodiments, the ICI is an anti-CD24 antibody, an anti-CD47 antibody, an anti-CD40 antibody, an anti-PD-1 antibody, an anti-CTLA-4 antibody, an anti-PD-L1 antibody, or an anti-PD-L2 antibody, or a functional fragment or functional derivative thereof. In some embodiments, the immune checkpoint inhibitor is nivolumab, pembrolizumab, atezolizumab, durvalumab, pidilizumab, PDR001, BMS-936559, avelumab, magrolimab, TTI-621, TTI-622, or SHR-1210.

In some embodiments, the cancer cells are a cancer cell line. In some embodiments, the cancer cell line is lung cell line KP-238N1 or lung cancer cell line KP-368T1.

In some embodiments, the targetable endonuclease is a Cas protein or functional fragment or functional derivative thereof. The Cas protein or functional fragment or derivative thereof is not limited and may be any suitable Cas protein or functional fragment or derivative having a desired activity. Specific examples of Cas proteins include Cas1, Cas2, Cas3, Cas4, Cas5, Cas6, Cas7, Cas8, Cas9 and Cas10. In a particular aspect, the Cas nucleic acid or protein used in the methods is Cas9. In some embodiments a Cas protein, e.g., a Cas9 protein, may be from any of a variety of prokaryotic species. In some embodiments a particular Cas protein, e.g., a particular Cas9 protein, may be selected to recognize a particular protospacer-adjacent motif (PAM) sequence. In certain embodiments a Cas protein, e.g., a Cas9 protein, may be obtained from a bacteria or archaea or synthesized using known methods. In certain embodiments, a Cas protein may be from a gram-positive bacteria or a gram-negative bacteria. In certain embodiments, a Cas protein may be from a Streptococcus, (e.g., a S. pyogenes, a S. thermophilus) a Cryptococcus, a Corynebacterium, a Haemophilus, a Eubacterium, a Pasteurella, a Prevotella, a VeiUonella, or a Marinobacter. In some embodiments nucleic acids encoding two or more different Cas proteins, or two or more Cas proteins, may be introduced into a cell to allow for recognition and modification of sites comprising the same, similar or different PAM motifs.

In some embodiments, the Cas protein is Cpf1 protein or a functional portion or derivative thereof. In some embodiments, the Cas protein is Cpf1 from any bacterial species or functional portion thereof. In certain embodiments, a Cpf1 protein is a Francisella novicida U112 protein or a functional portion thereof, an Acidaminococcus sp. BV3L6 protein or a functional portion thereof, or a Lachnospiraceae bacterium ND2006 protein or a function portion thereof. Cpf1 protein is a member of the type V CRISPR systems. Cpf1 protein is a polypeptide comprising about 1300 amino acids. Cpf1 contains a RuvC-like endonuclease domain.

In some embodiments, the cas is has a nuclease-dead RNA-guided DNA binding domain, e.g., dCas, optionally tethered to transcriptional repressor domains that promote epigenetic silencing (e.g., KRAB), to be used for “CRISPRi” transcription repression. In some embodiments, a dCas and a guide RNA is engineered to carry RNA binding motifs (e.g., MS2) that recruit effector domains fused to RNA-motif binding proteins, increasing transcription (CRISPRa).

In some embodiments, the Cas protein or functional fragment thereof comprises a detectable label. The term “detectable tag” or “detectable label” as used herein includes, but is not limited to, detectable labels, such as fluorophores, radioisotopes, colorimetric substrates, or enzymes; heterologous epitopes for which specific antibodies are commercially available, e.g., FLAG-tag; heterologous amino acid sequences that are ligands for commercially available binding proteins, e.g., Strep-tag, biotin; fluorescence quenchers typically used in conjunction with a fluorescent tag on the other polypeptide; and complementary bioluminescent or fluorescent polypeptide fragments. A tag that is a detectable label or a complementary bioluminescent or fluorescent polypeptide fragment may be measured directly (e.g., by measuring fluorescence or radioactivity of, or incubating with an appropriate substrate or enzyme to produce a spectrophotometrically detectable color change for the associated polypeptides as compared to the unassociated polypeptides). A tag that is a heterologous epitope or ligand is typically detected with a second component that binds thereto, e.g., an antibody or binding protein, wherein the second component is associated with a detectable label. In some embodiments, the detectable tag is a fluorescent tag.

In some embodiments, expression of the Cas protein or functional fragment thereof is under control of an inducible promoter or constitutive promoter and/or wherein expression of the sgRNAs in the library are under control of an inducible promoter or constitutive promoter. The term “inducible promoter,” as used herein, refers to a promoter that, in the absence of an inducer (such as a chemical and/or biological agent), does not direct expression, or directs low levels of expression of an operably linked gene (including cDNA), and, in response to an inducer, its ability to direct expression is enhanced. Exemplary inducible promoters include, for example, promoters that respond to heavy metals (CRC Boca Raton, Fla. (1991), 167-220; Brinster et al. Nature (1982), 296, 39-42), to thermal shocks, to hormones (Lee et al. P.N.A.S. USA (1988), 85, 1204-1208; (1981), 294, 228-232; Klock et al. Nature (1987), 329, 734-736; Israel and Kaufman, Nucleic Acids Res. (1989), 17, 2589-2604), promoters that respond to chemical agents, such as glucose, lactose, galactose or antibiotic (e.g., tetracycline or doxycycline).

In some specific embodiments, expression of the Cas protein or functional fragment thereof is induced with a site-specific recombinase. In some specific embodiments, expression of the plurality of sgRNAs are induced with a site-specific recombinase. The term “site-specific recombinase” (also referred to simply as a “recombinase” herein) refers to a protein that can recognize and catalyze the recombination of DNA between specific sequences in a DNA molecule. Such sequences may be referred to as “recombination sequences” or “recombination sites” for that particular recombinase. Tyrosine recombinases and serine recombinases are the two main families of site-specific recombinase. Examples of site-specific recombinase systems include the Cre/Lox system (Cre recombinase mediates recombination between loxP), the Flp/Frt system (Flp recombinase mediates recombination between FRT sites), and the PhiC31 system (PhiC31 recombinase mediates DNA recombination at sequences known as attB and attP sites). Recombinase systems similar to Cre include the Dre-rox, VCre/VloxP, and SCre/SloxP systems (Anastassiadis K, et al. (2009) Dis Model Mech 2(9-10):508-515; Suzuki E, Nakayama M (2011) Nucl. Acids Res. (2011) 39 (8): e49. It should be understood that reference to a particular recombinase system is intended to encompass the various engineered and mutant forms of the recombinases and recombination sites and codon-optimized forms of the coding sequences known in the art. DNA placed between two loxP sites is said to be “floxed”. A gene may be modified by the insertion of two loxP sites that allow the excision of the floxed gene segment through Cre-mediated recombination. In some embodiments, expression of Cre may be under control of a cell type specific, cell state specific, or inducible expression control element (e.g., cell type specific, cell state specific, or inducible promoter) or Cre activity may be regulated by a small molecule. For example, Cre may be fused to a ligand binding domain of a receptor (e.g., a steroid hormone receptor) so that its activity is regulated by receptor ligands. Cre-ER(T) or Cre-ER(T2) recombinases may be used, which comprise a fusion protein between a mutated ligand binding domain of the human estrogen receptor (ER) and Cre, the activity of which can be induced by, e.g., 4-hydroxy-tamoxifen. Placing Lox sequences appropriately allows a variety of genomic manipulations. In some embodiments, a nucleotide sequence coding for the site-specific recombinase (e.g., Cre) is introduced into the cell. In some embodiments, a nucleotide sequence coding for the site-specific recombinase (e.g., Cre) is introduced with a viral vector (e.g., AAV vector).

In some embodiments, the targetable nuclease is expressed in the cell. In some embodiments, the targetable nuclease protein is electroporated or otherwise transduced into the cell.

sgRNAs are known in the art. sgRNA refers to a single, contiguous RNA sequence that interacts with a cognate Cas protein equivalently as described for tracrRNA/crRNA polynucleotides. For example, a Cas9 single-guide RNA is a guide RNA wherein the Cas9-crRNA is covalently joined to the Cas9-tracrRNA, often through a tetraloop, and forms an RNA polynucleotide secondary structure through base-pair hydrogen bonding. See, e.g., Jinek, et al, Science (2012) 337:816-821; PCT Publication No. WO 2013/176772, published Nov. 28, 2013; (each of which is incorporated herein by reference in its entirety).

In some embodiments, the population of cancer cells of step a) has been transduced with a pool of sgRNA targeting at least 80%, 85%, 90%, 95%, 97%, 99%, 99.9%, 99.99%, 99.995%, or 99.999% of all the genes expressed on the cell surface in the genome of the cancer cell. In some embodiments, the population of cancer cells of step a) has been transduced with a pool of sgRNA targeting every gene expressed on the cell surface in the genome of the cancer cell. In some embodiments, the population of cancer cells of step a) has been transduced with a pool of sgRNA at a multiplicity of infection (MOI—ratio of gRNA to cancer cells) of 0.1 to 0.6. In some embodiments, the population of cancer cells of step a) has been transduced with a pool of sgRNA targeting every gene expressed on the cell surface in the genome of the cancer cell at a multiplicity of infection of about 0.2 to 0.4. In some embodiments, the population of cancer cells of step a) has been transduced with a pool of sgRNA targeting every gene expressed on the cell surface in the genome of the cancer cell at a multiplicity of infection of about 0.3.

In some embodiments, the population of cancer cells of step a) has been transduced with a pool of sgRNA targeting at least 80%, 85%, 90%, 95%, 97%, 99%, 99.9%, 99.99%, 99.995%, or 99.999% of all the genes expressed in the genome of the cancer cell. In some embodiments, the population of cancer cells of step a) has been transduced with a pool of sgRNA targeting every gene in the genome of the cancer cell. In some embodiments, the population of cancer cells of step a) has been transduced with a pool of sgRNA at a multiplicity of infection (MOI—ratio of gRNA to cancer cells) of 0.1 to 0.6. In some embodiments, the population of cancer cells of step a) has been transduced with a pool of sgRNA targeting every gene in the genome of the cancer cell at a multiplicity of infection of about 0.2 to 0.4. In some embodiments, the population of cancer cells of step a) has been transduced with a pool of sgRNA targeting every gene in the genome of the cancer cell at a multiplicity of infection of about 0.3.

The method of transduction is not limited. In some embodiment, transduction is via dextran-mediated transfection, calcium phosphate precipitation, polybrene mediated transfection, protoplast fusion, electroporation, and encapsulation in liposomes. In some embodiments, transduction is via electroporation. In some embodiments, transduction is via liposomes.

In some embodiments, least one sgRNA target each gene of the targeted genes. In some embodiments, least about two sgRNA target each gene of the targeted genes. In some embodiments, least about three sgRNA target each gene of the targeted genes. In some embodiments, least about four sgRNA target each gene of the targeted genes.

The myeloid cell type is not limited and may be any suitable myeloid cell type. In some embodiments, the myeloid cells are granulocytes, monocytes, macrophages, or dendritic cells. In some embodiments, the myeloid cells are macrophages. In some embodiments, the macrophages are polarized macrophages, M1 macrophages, M2 macrophages, or tumor-associated macrophages. In some embodiments, the macrophages are polarized macrophages. In some embodiments, the macrophages are M1 polarized macrophages. In some embodiments, the macrophages are M2 polarized macrophages. In some embodiments, the macrophages are tumor-associated macrophages. In some embodiments, the macrophages are resident macrophages. Resident macrophages include but are not limited to splenic macrophages, Kupffer cells, microglia, alveolar macrophages, pleural macrophages, peritoneal macrophages, osteoclasts, and histiocytes.

In some embodiments, the cancer cells and myeloid cells are cocultured for at least 1 minute, 5 minutes, 10 minutes, 30 minutes, 1 hour, 2 hours, 6 hours, 12 hours, 18 hours, or 1, 2, 3, 4, 5, 6, 7, 8 or more days in step c). In some embodiments, the cancer cells and myeloid cells are cocultured for 1 day or more in step c). In some embodiments, the cancer cells and myeloid cells are cocultured for about 1 day in step c).

In some embodiments, an increased abundance of sgRNA targeting a gene as compared to the control (e.g., identical cancer cells not contacted with myeloid cells) indicates that the product of the gene enhances myeloid cell-mediated killing of the cancer cells (e.g., an “Eat Me” or “EM” signal)—in other words, the sgRNA knocks out the “Eat Me” signal and promotes survival of a cancer cell having that sgRNA. In some embodiments, a decreased abundance of sgRNA targeting a gene as compared to the control indicates that the product of the gene inhibits myeloid cell-mediated killing of the cancer cells (e.g., a “Don't Eat Me” or “DME” signal)—in other words, the sgRNA knocks out the “Don't Eat Me” signal and inhibits survival of a cancer cell having that sgRNA.

In some embodiments, a cancer cell gene is identified as an EM signal when the gene has a positive “average phenotype of strongest 3” and a discovery score (discScore) of greater than about 3, about 4, or about 5. The discovery score can be calculated as follows: discScore=−log 10 (Mann-Whitney p value)*z-scored log 2Fold change. As is apparent, the discScore takes into consideration of both phenotype difference and statistical significance when selecting hits. “Average phenotype of the strongest 3” is wherein each gene is targeted by 4-5 sgRNA and the top 3 sgRNAs that give the strongest phenotype are picked. The average log 2FoldChange for the strongest 3 is then used for the “average phenotype of strongest 3” value. In some embodiments, a cancer cell gene is identified as an EM signal when the gene has a positive “average phenotype of strongest 3” and a discovery score (discScore) of greater than or equal to 5 (i.e., an FDR<5%). The mathematical basis and the analytical pipeline of this analysis is described in Horlbeck et al., eLife 2016; 5:e19760 DOI: 10.7554/eLife.19760, incorporated herein by reference.

In some embodiments, a cancer cell gene is identified as a DEM signal when the gene has a negative “average phenotype of strongest 3” and a discovery score (discScore) of greater than about 3, about 4, or about 5. In some embodiments, a cancer cell gene is identified as a DEM signal when the gene has a negative “average phenotype of strongest 3” and a discovery score (discScore) of greater than or equal to 5.

Methods of detecting the relative abundance of each sgRNA are not limited and may be any suitable method. In some embodiments, the relative abundance of each gRNA is detected by sequencing, e.g., next generation sequencing (NGS). In some embodiments, the sequencing method is multiplexed PCR-based NGS. In some embodiments, the sequencing method is a comprehensive amplicon NGS.

Next-generation sequencing technologies can include any one or more of high-throughput sequencing (e.g., facilitated through high-throughput sequencing technologies; massively parallel signature sequencing, Polony sequencing, 454 pyrosequencing, Illumina sequencing, SOLiD sequencing, Ion Torrent semiconductor sequencing and/or other suitable semiconductor-based sequencing technologies, DNA nanoball sequencing, Heliscope single molecule sequencing, Single molecule real time (SMRT) sequencing, Nanopore DNA sequencing, etc.), any generation number of sequencing technologies (e.g., second-generation sequencing technologies, third-generation sequencing technologies, fourth-generation sequencing technologies, etc.), sequencing-by-synthesis, tunneling currents sequencing, sequencing by hybridization, mass spectrometry sequencing, microscopy-based techniques, and/or any suitable next-generation sequencing technologies.

Additionally or alternatively, sequencing technologies can include any one or more of: capillary sequencing, Sanger sequencing (e.g., microfluidic Sanger sequencing, etc.), pyrosequencing, nanopore sequencing (Oxford nanopore sequencing, etc.), and/or any other suitable types of sequencing facilitated by any suitable sequencing technologies.

In some alternate embodiments of the methods disclosed herein, instead of using a targetable endonuclease and an sgRNA library targeting genes, RNA interference is used. The term “RNA interference” (RNAi) encompasses processes in which a molecular complex known as an RNA-induced silencing complex (RISC) reduces gene expression in a sequence-specific manner. RISC may incorporate a short nucleic acid strand (e.g., about 16- about 30 nucleotides (nt) in length) that pairs with and directs or “guides” sequence-specific degradation or translational repression of RNA (e.g., mRNA) to which the strand has complementarity. The short nucleic acid strand may be referred to as a “guide strand” or “antisense strand”. An RNA strand to which the guide strand has complementarity may be referred to as a “target RNA.” A guide strand may initially become associated with RISC components (in a complex sometimes termed the RISC loading complex) as part of a short double-stranded RNA (dsRNA), e.g., a short interfering RNA (siRNA). The other strand of the short dsRNA may be referred to as a “passenger strand” or “sense strand”. The complementarity of the structure formed by hybridization of a target RNA and the guide strand may be such that the strand can (i) guide cleavage of the target RNA in the RNA-induced silencing complex (RISC) and/or (ii) cause translational repression of the target RNA. Reduction of expression due to RNAi may be essentially complete (e.g., the amount of a gene product is reduced to background levels) or may be less than complete in various embodiments. For example, mRNA and/or protein level may be reduced by 50%, 60%, 70%, 75%, 80%, 85%, 90%, 95%, or more, in various embodiments. As known in the art, the complementarity between the guide strand and a target RNA need not be perfect (100%) but need only be sufficient to result in inhibition of gene expression. For example, in some embodiments 1, 2, 3, 4, 5, or more nucleotides of a guide strand may not be matched to a target RNA. “Not matched” or “unmatched” refers to a nucleotide that is mismatched (not complementary to the nucleotide located opposite it in a duplex, i.e., wherein Watson-Crick base pairing does not take place) or forms at least part of a bulge. Examples of mismatches include, without limitation, an A opposite a G or A, a C opposite an A or C, a U opposite a C or U, a G opposite a G. A bulge refers to a sequence of one or more nucleotides in a strand within a generally duplex region that are not located opposite to nucleotide(s) in the other strand. “Partly complementary” refers to less than perfect complementarity. In some embodiments a guide strand has at least about 80%, 85%, or 90%, e.g., at least about 91%, 92%, 93%, 94%, 95%, 96%, 97%, 98%, 99%, or 100% sequence complementarity to a target RNA over a continuous stretch of at least about 15 nt, e.g., between 15 nt and 30 nt, between 17 nt and 29 nt, between 18 nt and 25 nt, between 19 nt and 23 nt, of the target RNA. In some embodiments at least the seed region of a guide strand (the nucleotides in positions 2-7 or 2-8 of the guide strand) is perfectly complementary to a target RNA. In some embodiments, a guide strand and a target RNA sequence may form a duplex that contains no more than 1, 2, 3, or 4 mismatched or bulging nucleotides over a continuous stretch of at least 10 nt, e.g., between 10-30 nt. In some embodiments a guide strand and a target RNA sequence may form a duplex that contains no more than 1, 2, 3, 4, 5, or 6 mismatched or bulging nucleotides over a continuous stretch of at least 12 nt, e.g., between 10-30 nt. In some embodiments, a guide strand and a target RNA sequence may form a duplex that contains no more than 1, 2, 3, 4, 5, 6, 7, or 8 mismatched or bulging nts over a continuous stretch of at least 15 nt, e.g., between 10-30 nt. In some embodiments, a guide strand and a target RNA sequence may form a duplex that contains no mismatched or bulging nucleotides over a continuous stretch of at least 10 nt, e.g., between 10-30 nt. In some embodiments, between 10-30 nt is 10, 11, 12, 13, 14, 15, 16, 17, 18, 19, 20, 21, 22, 23, 24, 25, 26, 27, 28, 29, or 30 nt.

As used herein, the term “RNAi agent” encompasses nucleic acids that can be used to achieve RNAi in eukaryotic cells. Short interfering RNA (siRNA), short hairpin RNA (shRNA), and microRNA (miRNA) are examples of RNAi agents. siRNAs typically comprise two separate nucleic acid strands that are hybridized to each other to form a structure that contains a double stranded (duplex) portion at least 15 nt in length, e.g., about 15- about 30 nt long, e.g., between 17-27 nt long, e.g., between 18-25 nt long, e.g., between 19-23 nt long, e.g., 15, 16, 17, 18, 19, 20, 21, 22, 23, 24, 25, 26, 27, 28, 29, or 30 nucleotides. In some embodiments the strands of an siRNA are perfectly complementary to each other within the duplex portion. In some embodiments the duplex portion may contain one or more unmatched nucleotides, e.g., one or more mismatched (non-complementary) nucleotide pairs or bulged nucleotides. In some embodiments either or both strands of an siRNA may contain up to about 1, 2, 3, or 4 unmatched nucleotides within the duplex portion. In some embodiments a strand may have a length of between 15-35 nt, e.g., between 17-29 nt, e.g., 19-25 nt, e.g., 21-23 nt. Strands may be equal in length or may have different lengths in various embodiments. In some embodiments, strands may differ by 1-10 nt in length. A strand may have a 5′ phosphate group and/or a 3′ hydroxyl (—OH) group. Either or both strands of an siRNA may comprise a 3′ overhang of, e.g., about 1-10 nt (e.g., 1-5 nt, e.g., 2 nt). Overhangs may be the same length or different in lengths in various embodiments. In some embodiments an overhang may comprise or consist of deoxyribonucleotides, ribonucleotides, or modified nucleotides or modified ribonucleotides such as 2′-O-methylated nucleotides, or 2′-O-methyl-uridine. An overhang may be perfectly complementary, partly complementary, or not complementary to a target RNA in a hybrid formed by the guide strand and the target RNA in various embodiments.

shRNAs are nucleic acid molecules that comprise a stem-loop structure and a length typically between about 40-150 nt, e.g., about 50-100 nt, e.g., about 60-80 nt. A “stem-loop structure” (also referred to as a “hairpin” structure) refers to a nucleic acid having a secondary structure that includes a region of nucleotides which are known or predicted to form a double strand (stem portion; duplex) that is linked on one side by a region of (usually) predominantly single-stranded nucleotides (loop portion). Such structures are well known in the art and the term is used consistently with its meaning in the art. A guide strand sequence may be positioned in either arm of the stem, i.e., 5ÿ with respect to the loop or 3ÿ with respect to the loop in various embodiments. As is known in the art, the stem structure does not require exact base-pairing (perfect complementarity). Thus, the stem may include one or more unmatched residues or the base-pairing may be exact, i.e., it may not include any mismatches or bulges. In some embodiments the stem is between 15-30 nt, e.g., between 17-29 nt, e.g., between 19-25 nt. In some embodiments the stem is between 15-19 nt. In some embodiments the stem is between 19-30 nt. The primary sequence and number of nucleotides within the loop may vary. Examples of loop sequences include, e.g., UGGU; ACUCGAGA; UUCAAGAGA. In some embodiments a loop sequence found in a naturally occurring miRNA precursor molecule (e.g., a pre-miRNA) may be used. In some embodiments a loop sequence may be absent (in which case the termini of the duplex portion may be directly linked). In some embodiments a loop sequence may be at least partly self-complementary. In some embodiments the loop is between 1 and 20 nt in length, e.g., 1-15 nt, e.g., 4-9 nt. The shRNA structure may comprise a 5′ or 3′ overhang. As known in the art, an shRNA may undergo intracellular processing, e.g., by the ribonuclease (RNase) III family enzyme known as Dicer, to remove the loop and generate an siRNA.

Mature endogenous miRNAs are short (typically 18-24 nt, e.g., about 22 nt), single-stranded RNAs that are generated by intracellular processing from larger, endogenously encoded precursor RNA molecules termed miRNA precursors (see, e.g., Bartel, D., Cell. 116(2):281-97 (2004); Bartel D P. Cell. 136(2):215-33 (2009); Winter, J., et al., Nature Cell Biology 11: 228-234 (2009). Artificial miRNA may be designed to take advantage of the endogenous RNAi pathway in order to silence a target RNA of interest. The sequence of such artificial miRNA may be selected so that one or more bulges is present when the artificial miRNA is hybridized to its target sequence, mimicking the structure of naturally occurring miRNA:mRNA hybrids. Those of ordinary skill in the art are aware of how to design artificial miRNA.

An RNAi agent that contains a strand sufficiently complementary to an RNA of interest so as to result in reduced expression of the RNA of interest (e.g., as a result of degradation or repression of translation of the RNA) in a cell or in an in vitro system capable of mediating RNAi and/or that comprises a sequence that is at least 80%, 90%, 95%, or more (e.g., 100%) complementary to a sequence comprising at least 10, 12, 15, 17, or 19 consecutive nucleotides of an RNA of interest may be referred to as being “targeted to” the RNA of interest. An RNAi agent targeted to an RNA transcript may also be considered to be targeted to a gene from which the transcript is transcribed.

In some embodiments an RNAi agent is a vector (e.g., an expression vector) suitable for causing intracellular expression of one or more transcripts that give rise to a siRNA, shRNA, or miRNA in the cell. Such a vector may be referred to as an “RNAi vector”. An RNAi vector may comprise a template that, when transcribed, yields transcripts that may form a siRNA (e.g., as two separate strands that hybridize to each other), shRNA, or miRNA precursor (e.g., pri-miRNA or pre-mRNA).

Antisense oligonucleotides (ASO) are small sequences of DNA or RNA (e.g., about 8-50 base pairs in length) able to target RNA transcripts by Watson-Crick base pairing, resulting in reduced or modified protein expression. In some embodiments, oligonucleotides are unmodified. In other embodiments oligonucleotides include one or more modifications, e.g., to improve solubility, binding, potency, and/or stability of the antisense oligonucleotide. Modified oligonucleotides may comprise at least one modification relative to unmodified RNA or DNA. In some embodiments, oligonucleotides are modified to include internucleoside linkage modifications, sugar modifications, and/or nucleobase modifications. Examples of such modifications are known to those of skill in the art.

In some embodiments the oligonucleotide is modified by the substitution of at least one nucleotide with a modified nucleotide, such that in vivo stability is enhanced as compared to a corresponding unmodified oligonucleotide. In some aspects, the modified nucleotide is a sugar-modified nucleotide. In another aspect, the modified nucleotide is a nucleobase-modified nucleotide.

In some embodiments, oligonucleotides, may contain at least one modified nucleotide analogue. The nucleotide analogues may be located at positions where the target-specific activity, e.g., the splice site selection modulating activity is not substantially affected, e.g., in a region at the 5′-end and/or the 3′-end of the oligonucleotide molecule. In some aspects, the ends may be stabilized by incorporating modified nucleotide analogues.

In some aspects preferred nucleotide analogues include sugar- and/or backbone-modified ribonucleotides (i.e., include modifications to the phosphate-sugar backbone). For example, the phosphodiester linkages of a ribonucleotide may be modified to include at least one of a nitrogen or sulfur heteroatom. In preferred backbone-modified ribonucleotides the phosphoester group connecting to adjacent ribonucleotides is replaced by a modified group, e.g., of phosphothioate group. In preferred sugar-modified ribonucleotides, the 2′ OH-group is replaced by a group selected from H, OR, R, halo, SH, SR, NH2, NHR, NR2 or ON, wherein R is C₁-C₆ alkyl, alkenyl or alkynyl and halo is F, Cl, Br or I.

In some embodiments, modified oligonucleotides comprise one or more modified nucleosides comprising a modified sugar moiety.

Modified oligonucleotides may comprise one or more nucleosides comprising an unmodified nucleobase. In some embodiments modified oligonucleotides comprise one or more nucleosides comprising a modified nucleobase. In some embodiments, modified oligonucleotides comprise one or more nucleosides that does not comprise a nucleobase.

In some embodiments, nucleosides of modified oligonucleotides are linked together using any internucleoside linkage. Additional modifications are known by those of skill in the art and examples can be found in WO 2019/241648, U.S. Pat. Nos. 10,307,434, 9,045,518, and 10,266,822, each of which is incorporated herein by reference.

In some alternate aspects, the cancer cell is replaced with a target cell. For example, it may be desirable to modulate myeloid cell-mediated killing of allogenic cells, IPSC derived cells, damaged cells, or cells of particular cell types such as neuronal cells, pre-cancerous cells, liver cells, etc. In some embodiments, target cells are abnormally reactive and/or autoantibody-secreting plasma cells and/or B cells. Depletion of such cells (e.g., by myeloid cell-mediated killing) may be useful in the treatment of a wide variety of autoimmune diseases, such as systemic lupus erythematosus (SLE), rheumatoid arthritis (RA), Sjogren's syndrome, or other autoimmune diseases, e.g., any autoimmune disease characterized by production of autoantibodies. In some embodiments such cells express BCMA, which can be a target of opsonizing antibodies.

Thus, some alternate aspects of the present disclosure are directed to a method of screening for a modulator of myeloid cell-mediated killing of target cells, comprising a) providing a population of target cells expressing a targetable endonuclease and an sgRNA library targeting genes or an interfering RNA library, b) contacting the target cells with myeloid cells capable of having an anti-target cell response, c) coculturing the target cells and the myeloid cells, and d) measuring the relative abundance of each sgRNA of the sgRNA library or interfering RNA of the interfering RNA library in the cocultured target cells as compared to the abundance of each in control target cells not contacted with the myeloid cells, wherein the differential relative abundance of an sgRNA or interfering RNA as compared to the control indicates that the gene targeted by the sgRNA or interfering RNA is a candidate modulator of myeloid cell-mediated killing of the target cells. The target cell is not limited and may be any suitable cell. In some embodiments, the target cell is muscle cell, brain cell, neuronal cell, liver cell, kidney cell, digestive tract cell, bone cell, cartilage cell, heart cell, lung cell, infected cell, fetal cell, endocrine cell, lymphatic cell, or epidermal cell.

Some aspects of the present disclosure are directed to a method of screening for an agent that increases MMCCK which comprises contacting a target or cancer cell with a test agent and detecting a change in the level or activity of a target or cancer cell gene identified as a EM signal or DME signal as compared to a control cell not contacted with the test agent, wherein a test agent that increases the level or activity an EM signal or decreases the level or activity of an DEM signal is identified as an agent that increases MMCCK. The test agent is not limited and may be any agent described herein. In some embodiments, the agent is a small molecule. In some embodiments, a reporter molecule, or a gene product activity (e.g., protease activity, kinase activity, enzymatic activity, etc) is used to detect whether a test agent increases MMCCK.

In some embodiments of the screening methods disclosed herein, a high throughput screen (HTS) is performed. High throughput screens often involve testing large numbers of compounds with high efficiency, e.g., in parallel. For example, tens or hundreds of thousands of compounds can be routinely screened in short periods of time, e.g., hours to days. Often such screening is performed in multiwell plates containing, at least 96 wells or other vessels in which multiple physically separated cavities or depressions are present in a substrate. High throughput screens often involve use of automation, e.g., for liquid handling, imaging, data acquisition and processing, etc. Certain general principles and techniques that may be applied in embodiments of a HTS of the present invention are described in Macarr6n R & Hertzberg R P. Design and implementation of high-throughput screening assays. Methods Mol Biol., 565:1-32, 2009 and/or An WF & Tolliday N J., Introduction: cell-based assays for high-throughput screening. Methods Mol Biol. 486:1-12, 2009, and/or references in either of these. Useful methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jorg Hüser.

The term “hit” generally refers to an agent that achieves an effect of interest in a screen or assay, e.g., an agent that has at least a predetermined level of modulating effect on cell survival, cell proliferation, gene expression, protein activity, or other parameter of interest being measured in the screen or assay. Test agents that are identified as hits in a screen may be selected for further testing, development, or modification. In some embodiments a test agent is retested using the same assay or different assays. Additional amounts of the test agent may be synthesized or otherwise obtained, if desired. Physical testing or computational approaches can be used to determine or predict one or more physicochemical, pharmacokinetic and/or pharmacodynamic properties of compounds identified in a screen. For example, solubility, absorption, distribution, metabolism, and excretion (ADME) parameters can be experimentally determined or predicted. Such information can be used, e.g., to select hits for further testing, development, or modification. For example, small molecules having characteristics typical of “drug-like” molecules can be selected and/or small molecules having one or more unfavorable characteristics can be avoided or modified to reduce or eliminated such unfavorable characteristic(s).

Additional compounds, e.g., analogs, that have a desired activity can be identified or designed based on compounds identified in a screen. In some embodiments structures of hit compounds are examined to identify a pharmacophore, which can be used to design additional compounds. An additional compound may, for example, have one or more altered, e.g., improved, physicochemical, pharmacokinetic (e.g., absorption, distribution, metabolism and/or excretion) and/or pharmacodynamic properties as compared with an initial hit or may have approximately the same properties but a different structure. For example, a compound may have higher affinity for the molecular target of interest, lower affinity for a non-target molecule, greater solubility (e.g., increased aqueous solubility), increased stability, increased bioavailability, oral bioavailability, and/or reduced side effect(s), modified onset of therapeutic action and/or duration of effect. An improved property is generally a property that renders a compound more readily usable or more useful for one or more intended uses. Improvement can be accomplished through empirical modification of the hit structure (e.g., synthesizing compounds with related structures and testing them in cell-free or cell-based assays or in non-human animals) and/or using computational approaches. Such modification can make use of established principles of medicinal chemistry to predictably alter one or more properties. An analog that has one or more improved properties may be identified and used in a composition or method described herein. In some embodiments a molecular target of a hit compound is identified or known. In some embodiments, additional compounds that act on the same molecular target may be identified empirically (e.g., through screening a compound library) or designed.

Data or results from testing an agent or performing a screen may be stored or electronically transmitted. Such information may be stored on a tangible medium, which may be a computer-readable medium, paper, etc. In some embodiments a method of identifying or testing an agent comprises storing and/or electronically transmitting information indicating that a test agent has one or more propert(ies) of interest or indicating that a test agent is a “hit” in a particular screen, or indicating the particular result achieved using a test agent. A list of hits from a screen may be generated and stored or transmitted. Hits may be ranked or divided into two or more groups based on activity, structural similarity, or other characteristics

Once a candidate agent is identified, additional agents, e.g., analogs, may be generated based on it. An additional agent, may, for example, have increased cell uptake, increased potency, increased stability, greater solubility, or any improved property. In some embodiments a labeled form of the agent is generated. The labeled agent may be used, e.g., to directly measure binding of an agent to a molecular target in a cell. In some embodiments, a molecular target of an agent identified as described herein may be identified. An agent may be used as an affinity reagent to isolate a molecular target. An assay to identify the molecular target, e.g., using methods such as mass spectrometry, may be performed. Once a molecular target is identified, one or more additional screens maybe performed to identify agents that act specifically on that target.

Any of a wide variety of agents may be used as a test agent in various embodiments. For example, a test agent may be a small molecule, polypeptide, peptide, amino acid, nucleic acid, oligonucleotide, lipid, carbohydrate, or hybrid molecule. In some embodiments a nucleic acid used as a test agent comprises a siRNA, shRNA, antisense oligonucleotide, aptamer, or random oligonucleotide. In some embodiments a test agent is cell permeable or provided in a form or with an appropriate carrier or vector to allow it to enter cells.

Agents can be obtained from natural sources or produced synthetically. Agents may be at least partially pure or may be present in extracts or other types of mixtures. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. A compound library may comprise natural products and/or compounds generated using non-directed or directed synthetic organic chemistry. In some embodiments a library is a small molecule library, peptide library, peptoid library, cDNA library, oligonucleotide library, or display library (e.g., a phage display library). In some embodiments a library comprises agents of two or more of the foregoing types. In some embodiments oligonucleotides in an oligonucleotide library comprise siRNAs, shRNAs, antisense oligonucleotides, aptamers, or random oligonucleotides.

A library may comprise, e.g., between 100 and 500,000 compounds, or more. In some embodiments a library comprises at least 10,000, at least 50,000, at least 100,000, or at least 250,000 compounds. In some embodiments compounds of a compound library are arrayed in multiwell plates. They may be dissolved in a solvent (e.g., DMSO) or provided in dry form, e.g., as a powder or solid. Collections of synthetic, semi-synthetic, and/or naturally occurring compounds may be tested. Compound libraries can comprise structurally related, structurally diverse, or structurally unrelated compounds. Compounds may be artificial (having a structure invented by man and not found in nature) or naturally occurring. In some embodiments compounds that have been identified as “hits” or “leads” in a drug discovery program and/or analogs thereof. In some embodiments a library may be focused (e.g., composed primarily of compounds having the same core structure, derived from the same precursor, or having at least one biochemical activity in common). Compound libraries are available from a number of commercial vendors such as Tocris BioScience, Nanosyn, BioFocus, and from government entities such as the U.S. National Institutes of Health (NIH). In some embodiments a test agent is not an agent that is found in a cell culture medium known or used in the art, e.g., for culturing vertebrate, e.g., mammalian cells, e.g., an agent provided for purposes of culturing the cells. In some embodiments, if the agent is one that is found in a cell culture medium known or used in the art, the agent may be used at a different, e.g., higher, concentration when used as a test agent in a method or composition described herein.

### Screening for ICI Enhancers/Inhibitors

Some aspects of the present disclosure are directed to a method of screening for a modulator of myeloid cell-mediated killing of cancer cells in the presence of a myeloid cell checkpoint inhibitor (also referred to myeloid cell immune checkpoint inhibitors, i.e., myeICI) or other anti-cancer agent, comprising a) providing a population of cancer cells expressing or otherwise comprising a targetable endonuclease and an sgRNA library targeting genes, b) contacting the cancer cells with myeloid cells capable of having an anticancer response and the myeloid cell checkpoint inhibitor or other anti-cancer agent, c) coculturing the cancer cells and myeloid cells with the myeloid cell checkpoint inhibitor or other anti-cancer agent, and d) measuring the relative abundance of each sgRNA of the sgRNA library in the cocultured cancer cells as compared to the abundance of each gRNA in control cancer cells cocultured with myeloid cells but not the myeloid cell checkpoint inhibitor or other anti-cancer agent, wherein the differential relative abundance of an sgRNA as compared to the control indicates that the gene targeted by the sgRNA is a candidate modulator of myeloid cell-mediated killing of cancer cells in the presence of the myeloid cell checkpoint inhibitor or other anti-cancer agent. In some alternate embodiments, the population of myeloid cells instead of the population of cancer cell (or target cell as detailed below) is provided with a targetable endonuclease and an sgRNA library targeting genes (or RNA interference library as disclosed herein).

As used herein “modulating” (and verb forms thereof, such as “modulates”) means causing or facilitating a qualitative or quantitative change, alteration, or modification. Without limitation, such change may be an increase or decrease in a qualitative or quantitative aspect.

The type of cancer cell is not limited and may be any cancer disclosed herein. In some embodiments, the cancer is a cancer responsive to an immune checkpoint inhibitor (ICI). In some embodiments, the cancer is resistant or has developed resistance to an immune checkpoint inhibitor. In some embodiments, the cancer cells are a cancer cell line. In some embodiments, the cancer cell line is lung cell line KP-238N1 or lung cancer cell line KP-368T1. The ICI is not limited and may be any ICI disclosed herein. In some embodiments, the ICI is an anti-CD24 antibody, an anti-CD47 antibody, an anti-CD40 antibody, an anti-PD-1 antibody, an anti-CTLA-4 antibody, an anti-PD-L1 antibody, or an anti-PD-L2 antibody or a functional fragment or functional derivative thereof.

The targetable endonuclease is not limited and may be any targetable endonuclease provided herein. In some embodiments, the targetable endonuclease is a Cas9. In some embodiments, the targetable nuclease is expressed in the cell. In some embodiments, the targetable nuclease protein is electroporated or otherwise transduced into the cell.

In some embodiments, the population of cancer cells of step a) has been transduced with a pool of sgRNA targeting at least 80%, 85%, 90%, 95%, 97%, 99%, 99.9%, 99.99%, 99.995%, or 99.999% of all the genes expressed on the cell surface in the genome of the cancer cell. In some embodiments, the population of cancer cells of step a) has been transduced with a pool of sgRNA targeting every gene expressed on the cell surface in the genome of the cancer cell. In some embodiments, the population of cancer cells of step a) has been transduced with a pool of sgRNA at a multiplicity of infection (MOI—ratio of gRNA to cancer cells) of 0.1 to 0.6. In some embodiments, the population of cancer cells of step a) has been transduced with a pool of sgRNA targeting every gene expressed on the cell surface in the genome of the cancer cell at a multiplicity of infection of about 0.2 to 0.4. In some embodiments, the population of cancer cells of step a) has been transduced with a pool of sgRNA targeting every gene expressed on the cell surface in the genome of the cancer cell at a multiplicity of infection of about 0.3.

In some embodiments, the population of cancer cells of step a) has been transduced with a pool of sgRNA targeting at least 80%, 85%, 90%, 95%, 97%, 99%, 99.9%, 99.99%, 99.995%, or 99.999% of all the genes expressed in the genome of the cancer cell. In some embodiments, the population of cancer cells of step a) has been transduced with a pool of sgRNA targeting every gene in the genome of the cancer cell. In some embodiments, the population of cancer cells of step a) has been transduced with a pool of sgRNA at a multiplicity of infection (MOI—ratio of gRNA to cancer cells) of 0.1 to 0.6. In some embodiments, the population of cancer cells of step a) has been transduced with a pool of sgRNA targeting every gene in the genome of the cancer cell at a multiplicity of infection of about 0.2 to 0.4. In some embodiments, the population of cancer cells of step a) has been transduced with a pool of sgRNA targeting every gene in the genome of the cancer cell at a multiplicity of infection of about 0.3.

The method of transduction is not limited. In some embodiment, transduction is via dextran-mediated transfection, calcium phosphate precipitation, polybrene mediated transfection, protoplast fusion, electroporation, and encapsulation in liposomes. In some embodiments, transduction is via electroporation. In some embodiments, transduction is via liposomes.

In some embodiments, least one sgRNA target each gene of the targeted genes. In some embodiments, least about two sgRNA target each gene of the targeted genes. In some embodiments, least about three sgRNA target each gene of the targeted genes. In some embodiments, least about four sgRNA target each gene of the targeted genes.

The myeloid cell type is not limited and may be any suitable myeloid cell type. In some embodiments, the myeloid cells are granulocytes, monocytes, macrophages, microglia, or dendritic cells. In some embodiments, the myeloid cells are macrophages. In some embodiments, the macrophages are polarized macrophages, M1 macrophages, M2 macrophages, or tumor-associated macrophages. In some embodiments, the macrophages are polarized macrophages. In some embodiments, the macrophages are M1 polarized macrophages. In some embodiments, the macrophages are M2 polarized macrophages. In some embodiments, the macrophages are tumor-associated macrophages. In some embodiments, the macrophages are resident macrophages. Resident macrophages include but are not limited to splenic macrophages, Kupffer cells, microglia, alveolar macrophages, pleural macrophages, peritoneal macrophages, osteoclasts, and histiocytes.

In some embodiments, the cancer cells and myeloid cells are cocultured (e.g., in the presence of the myeloid cell checkpoint inhibitor or other anti-cancer agent for at least 1, 2, 3, 4, 5, 6, 7, 8 or more days in step c). In some embodiments, the cancer cells and myeloid cells are cocultured with the myeloid cell checkpoint inhibitor or other anti-cancer agent for 1 day or more in step c). In some embodiments, the cancer cells and myeloid cells are cocultured with the myeloid cell checkpoint inhibitor or other anti-cancer agent for about 1 day in step c). In some embodiments, the cancer cells and myeloid cells are cocultured with the myeloid cell checkpoint inhibitor or other anti-cancer agent for 5 days or more in step c). In some embodiments, the cancer cells and myeloid cells are cocultured with the myeloid cell checkpoint inhibitor or other anti-cancer agent for about 5 days in step c).

The myeloid cell checkpoint inhibitor (MyeICI) is not limited and may be any suitable myeloid cell checkpoint inhibitor. In some embodiments, the myeloid cell checkpoint inhibitor is a CD24 antibody, a CD47 antagonist, a CD40 agonist, or a PD-L1 antagonist. In some embodiments, the MyeICI is an anti-CD24 antibody, an anti-CD47 antibody, an anti-CD40 antibody, an anti-PD-L1 antibody, or a functional fragment or functional derivative thereof.

The anti-cancer agent is not limited. In some embodiments, the anti-cancer agent is an immunotherapy agent. The immunotherapy agent is not limited. In some embodiments, the immunotherapy agent includes, but is not limited to, atezolizumab, avelumab, bavituximab, bevacizumab (avastin), bivatuzumab, blinatumomab, conatumumab, daratumumab, duligotumab, dacetuzumab, dalotuzumab, durvalumab, elotuzumab (HuLuc63), gemtuzumab, ibritumomab, indatuximab, inotuzumab, ipilimumab, lorvotuzumab, lucatumumab, milatuzumab, moxetumomab, nivolumab, ocaratuzumab, ofatumumab, pembrolizumab, rituximab, siltuximab, teprotumumab, and ublituximab.

In some embodiments, the anti-cancer agent is ionizing radiation.

In some embodiments, the anti-cancer agent is a chemotherapeutic agent. The chemotherapeutic agent is not limited. Chemotherapeutic agents useful in methods disclosed herein include, but are not limited to, alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, bendamustine, chlorozotocin, fotemustine, lomustine, nimustine, ranimustine; antibiotics such as aclacinomysins, actinomycin, authramycin, azaserine, bleomycins, dactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel and docetaxel; chlorambucil; gemcitabine; 6-thioguanine; mercaptopurine; platinum analogs such as cisplatin and carboplatin; vinblastine; platinum; etoposide; ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine; retinoic acid; esperamicins; capecitabine; and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide, and irinotecan, as well as pharmaceutically acceptable salts, acids, or derivatives of any of these. In some embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain e embodiments, the antimitotic agent comprises a vinca alkaloid, such as vincristine, binblastine, vinorelbine, or vindesine, or pharmaceutically acceptable salts, acids, or derivatives thereof.

In some embodiments, an increased abundance of sgRNA targeting a gene as compared to the control indicates that the product of the gene enhances myeloid cell-mediated killing of cancer cells in the presence of a myeloid cell checkpoint inhibitor or other anti-cancer agent. In some embodiments, a decreased abundance of sgRNA targeting a gene as compared to the control indicates that the product of the gene inhibits myeloid cell-mediated killing of cancer cells in the presence of a myeloid cell checkpoint inhibitor or other anti-cancer agent. In some embodiments, statistical significance is calculated by the Mann-Whitney test.

In some embodiments, the cancer cell gene is identified as a modulator of myeICI or other anti-cancer agent when the gene has a discovery score (discScore) of greater than about 3, about 4, or about 5. In some embodiments, the cancer cell gene is identified as a modulator of myeICI or other anti-cancer agent when the gene has a discovery score (discScore) of greater than or equal to 5.

In some embodiments, a cancer cell gene is identified as inhibiting the activity of the myeICI or other anti-cancer agent when the gene has a positive “average phenotype of strongest 3” and a discovery score (discScore) of greater than about 3, about 4, or about 5. In some embodiments, a cancer cell gene is identified as inhibiting the activity of the myeICI or other anti-cancer agent when the gene has a positive “average phenotype of strongest 3” and a discovery score (discScore) of greater than or equal to 5.

In some embodiments, a cancer cell gene is identified as enhancing or being synergistic with the MyeICI or other anti-cancer agent when the gene has a negative “average phenotype of strongest 3” and a discovery score (discScore) of greater than about 3, about 4, or about 5. In some embodiments, a cancer cell gene is identified as enhancing or being synergistic with the MyeICI or other anti-cancer agent when the gene has a negative “average phenotype of strongest 3” and a discovery score (discScore) of greater than or equal to 5.

In some alternate embodiments of the methods disclosed herein, instead of using a targetable endonuclease and an sgRNA library targeting genes, RNA interference is used as described herein.

In some alternate aspects, the cancer cell is replaced with a target cell as described herein. For example, it may be desirable to modulate myeloid cell-mediated killing of allogenic cells, IPSC derived cells, damaged cells, or cells of particular cell types such as neuronal cells, pre-cancerous cells, liver cells, etc. Thus, some alternate aspects of the present disclosure are directed to a method of screening for a modulator of myeloid cell-mediated killing of target cells in the presence of an anti-target cell agent, comprising a) providing a population of target cells expressing or otherwise comprising a targetable endonuclease and an sgRNA library targeting genes or an interference RNA library, b) contacting the target cells with myeloid cells capable of having an anti-target cell response and the anti-target cell agent, c) coculturing the target cells and myeloid cells with the agent, and d) measuring the relative abundance of each sgRNA of the sgRNA library or RNAi agent in the cocultured target cells as compared to the abundance of each gRNA or RNAi in control target cells cocultured with myeloid cells but not the agent, wherein the differential relative abundance of an sgRNA or RNAi agent as compared to the control indicates that the gene targeted by the sgRNA or RNAi agent is a candidate modulator of myeloid cell-mediated killing of target cells in the presence of the agent.

Some aspects of the present disclosure are directed to a method of screening for an agent that increases the activity of an MyeICI or other anti-cancer agent which comprises contacting a cancer cell with a test agent and detecting a change in the level or activity of a cancer cell gene identified as inhibiting or enhancing (synergizing) the activity of the MyeICI or other anti-cancer agent as compared to a control cell not contacted with the test agent, wherein a test agent that increases the level or activity an EM signal or decreases the level or activity of an DEM signal is identified as an agent that increases MMCCK. The test agent is not limited and may be any agent described herein. In some embodiments, the agent is a small molecule. The MyeICI or other anti-cancer agent is not limited and may be any MyeICI or other anti-cancer agent described herein. In some embodiments of the screening methods disclosed herein, a high throughput screen (HTS) is performed.

Methods of Treating Cancer with MMCCK Modulators

Some aspects of the present disclosure are directed to a method of treating cancer in a subject comprising administering to the subject an agent that modulates the level or activity of a cancer cell gene that modulates macrophage-mediated cancer cell killing (MMCCK).

The terms “treating” and “treatment” refer to administering to a subject an effective amount of an agent so that the subject experiences a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment.

The cancer is not limited and may be any cancer disclosed herein. In some embodiments, the cancer is a lung cancer.

The term “subject” and “patient” may be used herein interchangeably. The subject is not limited and may be any suitable subject. In some embodiments, the subject is a mammal. In some embodiments, the subject is a rat, mouse, human, or non-human primate.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In some embodiments, the agent is selected from the group consisting of a nucleic acid, a small molecule, a polypeptide, and a peptide. In some embodiments the agent is an oligonucleotide, protein, or a small molecule. In some embodiments the agent comprises one or more oligonucleotides. In some aspects the oligonucleotide is a splice-switching oligonucleotide. In certain aspects the oligonucleotide is an antisense oligonucleotide (ASO). In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds. In some embodiments, the agent is a genomic modification system (e.g., a CRISPR/Cas, Zinc Finger Nuclease, or TALEN systems). CRISPR/Cas systems can employ a variety of Cas proteins (Haft et al. PLoS Comput Biol. 2005; 1 (6)e60). In some embodiments, the CRISPR/Cas system is a CRISPR type I system. In some embodiments, the CRISPR/Cas system is a CRISPR type II system. In some embodiments, the CRISPR/Cas system is a CRISPR type V system.

“Small molecule” is defined as a molecule with a molecular weight that is less than 10 kD, typically less than 2 kD, and preferably less than 1 kD. Small molecules include, but are not limited to, inorganic molecules, organic molecules, organic molecules containing an inorganic component, molecules comprising a radioactive atom, synthetic molecules, peptide mimetics, and antibody mimetics. As a therapeutic, a small molecule may be more permeable to cells, less susceptible to degradation, and less apt to elicit an immune response than large molecules.

As used herein, the term “polypeptide” or “protein” is used to designate a series of amino acid residues connected to the other by peptide bonds between the alpha-amino and carboxy groups of adjacent residues. The term “polypeptide” refers to a polymer of protein amino acids, including modified amino acids (e.g., phosphorylated, glycated, glycosylated, etc.) and amino acid analogs, regardless of its size or function. The term “peptide” is often used in reference to small polypeptides, but usage of this term in the art overlaps with “protein” or “polypeptide.” Exemplary polypeptides include gene products, naturally occurring proteins, homologs, orthologs, paralogs, fragments and other equivalents, as well as both naturally and non-naturally occurring variants, fragments, and analogs of the foregoing.

The term “nucleic acid” refers to polynucleotides such as deoxyribonucleic acid (DNA) and ribonucleic acid (RNA). The terms “nucleic acid” and “polynucleotide” are used interchangeably herein and should be understood to include double-stranded polynucleotides, single-stranded (such as sense or antisense) polynucleotides, and partially double-stranded polynucleotides. A nucleic acid often comprises standard nucleotides typically found in naturally occurring DNA or RNA (which can include modifications such as methylated nucleobases), joined by phosphodiester bonds. In some embodiments a nucleic acid may comprise one or more non-standard nucleotides, which may be naturally occurring or non-naturally occurring (i.e., artificial; not found in nature) in various embodiments and/or may contain a modified sugar or modified backbone linkage. Nucleic acid modifications (e.g., base, sugar, and/or backbone modifications), non-standard nucleotides or nucleosides, etc., such as those known in the art as being useful in the context of RNA interference (RNAi), aptamer, CRISPR technology, polypeptide production, reprogramming, or antisense-based molecules for research or therapeutic purposes may be incorporated in various embodiments. Such modifications may, for example, increase stability (e.g., by reducing sensitivity to cleavage by nucleases), decrease clearance in vivo, increase cell uptake, or confer other properties that improve the translation, potency, efficacy, specificity, or otherwise render the nucleic acid more suitable for an intended use. Various non-limiting examples of nucleic acid modifications are described in, e.g., Deleavey G F, et al., Chemical modification of siRNA. Curr. Protoc. Nucleic Acid Chem. 2009; 39:16.3.1-16.3.22; Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008; U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929, 226; 5,977,296; 6,140,482; 6,455,308 and/or in PCT application publications WO 00/56746 and WO 01/14398. Different modifications may be used in the two strands of a double-stranded nucleic acid. A nucleic acid may be modified uniformly or on only a portion thereof and/or may contain multiple different modifications. Where the length of a nucleic acid or nucleic acid region is given in terms of a number of nucleotides (nt) it should be understood that the number refers to the number of nucleotides in a single-stranded nucleic acid or in each strand of a double-stranded nucleic acid unless otherwise indicated. An “oligonucleotide” is a relatively short nucleic acid, typically between about 5 and about 100 nt long.

In certain aspects, the agent is or comprises an antibody (e.g., a monoclonal or polyclonal antibody). The antibodies of the present invention can be polyclonal or monoclonal, and the term “antibody” is intended to encompass both polyclonal and monoclonal antibodies.

Antibodies of the present invention can be raised against an appropriate marker or antigen. Antibodies can be raised against a selected marker (e.g., a cell surface marker) or antigen by methods known to those skilled in the art. Such methods for raising polyclonal antibodies are well known in the art and are described in detail, for example, in Harlow et al., 1988 in: Antibodies, A Laboratory Manual, Cold Spring Harbor, NY.

Typically, such antibodies are raised by immunizing an animal (e.g. a rabbit, rat, mouse, donkey, etc.) by multiple subcutaneous or intraperitoneal injections of the relevant antigen optionally conjugated to keyhole limpet hemocyanin (KLH), serum albumin, other immunogenic carrier, diluted in sterile saline and combined with an adjuvant (e.g. Complete or Incomplete Freund's Adjuvant) to form a stable emulsion. The polyclonal antibody is then recovered from blood or ascites of the immunized animal. Collected blood is clotted, and the serum decanted, clarified by centrifugation, and assayed for antibody titer. The polyclonal antibodies can be purified from serum or ascites according to standard methods in the art including affinity chromatography, ion-exchange chromatography, gel electrophoresis, dialysis, etc. Polyclonal antiserum can also be rendered monospecific using standard procedures (see, e.g., Agaton et al., “Selective Enrichment of Monospecific Polyclonal Antibodies for Antibody-Based Proteomics Efforts,” J Chromatography A 1043(1):33-40 (2004), which is hereby incorporated by reference in its entirety).

In some embodiments, monoclonal antibodies can be prepared using hybridoma methods, such as those described by Kohler and Milstein, “Continuous Cultures of Fused Cells Secreting Antibody of Predefined Specificity,” Nature 256:495-7 (1975), which is hereby incorporated by reference in its entirety. Using the hybridoma method, a mouse, hamster, or other appropriate host animal, is immunized to elicit the production by lymphocytes of antibodies that will specifically bind to an immunizing antigen. Alternatively, lymphocytes can be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies can then be propagated either in vitro culture using standard methods (James Goding, Monoclonal Antibodies: Principles and Practice (1986) which is hereby incorporated by reference in its entirety) or in vivo as ascites tumors in an animal. The monoclonal antibodies can then be purified from the culture medium or ascites fluid as described for polyclonal antibodies above.

In some embodiments, monoclonal antibodies can be made using recombinant DNA methods as described in U.S. Pat. No. 4,816,567 to Cabilly et al., which is hereby incorporated by reference in its entirety. The polynucleotides encoding a monoclonal antibody are isolated, such as from mature B-cells or hybridoma cells, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, and monoclonal antibodies are generated by the host cells. Recombinant monoclonal antibodies or fragments thereof of the desired species can also be isolated from phage display libraries as described (McCafferty et al., “Phage Antibodies: Filamentous Phage Displaying Antibody Variable Domains,” Nature 348:552-554 (1990); Clackson et al., “Making Antibody Fragments using Phage Display Libraries,” Nature 352:624-628 (1991); and Marks et al., “By-Passing Immunization. Human Antibodies from V-Gene Libraries Displayed on Phage,” J. Mol. Biol. 222:581-597 (1991), which are hereby incorporated by reference in their entirety).

The polynucleotides encoding a monoclonal antibody can further be modified in a number of different ways using recombinant DNA technology to generate alternative antibodies. In one embodiment, the constant domains of the light and heavy chains of, for example, a mouse monoclonal antibody can be substituted for those regions of a human antibody to generate a chimeric antibody. Alternatively, the constant domains of the light and heavy chains of a mouse monoclonal antibody can be substituted for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity and affinity of a monoclonal antibody.

Humanized antibodies can be produced using various techniques known in the art. An antibody can be humanized by substituting the complementarity determining region (CDR) of a human antibody with that of a non-human antibody (e.g. mouse, rat, rabbit, hamster, etc.) having the desired specificity, affinity, and capability (Jones et al., “Replacing the Complementarity-Determining Regions in a Human Antibody With Those From a Mouse,” Nature 321:522-525 (1986); Riechmann et al., “Reshaping Human Antibodies for Therapy,” Nature 332:323-327 (1988); Verhoeyen et al., “Reshaping Human Antibodies: Grafting an Antilysozyme Activity,” Science 239:1534-1536 (1988), which are hereby incorporated by reference in their entirety). The humanized antibody can be further modified by the substitution of additional residues either in the Fv framework region and/or within the replaced non-human residues to refine and optimize antibody specificity, affinity, and/or capability.

Human antibodies can be directly prepared using various techniques known in the art. Immortalized human B lymphocytes immunized in vitro or isolated from an immunized individual that produces an antibody directed against a target antigen can be generated (see, e.g. Reisfeld et al., Monoclonal Antibodies and Cancer Therapy 77 (Alan R. Liss 1985) and U.S. Pat. No. 5,750,373 to Garrard, which are hereby incorporated by reference in their entirety). Also, the human antibody can be selected from a phage library, where that phage library expresses human antibodies (Vaughan et al., “Human Antibodies with Sub-Nanomolar Affinities Isolated from a Large Non-immunized Phage Display Library,” Nature Biotechnology, 14:309-314 (1996); Sheets et al., “Efficient Construction of a Large Nonimmune Phage Antibody Library: The Production of High-Affinity Human Single-Chain Antibodies to Protein Antigens,” Proc Nat'l Acad Sci USA 95:6157-6162 (1998); Hoogenboom et al., “By-passing Immunisation. Human Antibodies From Synthetic Repertoires of Germline V H Gene Segments Rearranged In Vitro,” J Mol. Biol, 227:381-8 (1992); Marks et al., “By-passing Immunization. Human Antibodies from V-gene Libraries Displayed on Phage,” J. Mol. Biol, 222:581-97 (1991), which are hereby incorporated by reference in their entirety). Humanized antibodies can also be made in transgenic mice containing human immunoglobulin loci that are capable upon immunization of producing the full repertoire of human antibodies in the absence of endogenous immunoglobulin production. This approach is described in U.S. Pat. No. 5,545,807 to Surani et al.; U.S. Pat. No. 5,545,806 to Lonberg et al.; U.S. Pat. No. 5,569,825 to Lonberg et al.; U.S. Pat. No. 5,625,126 to Lonberg et al.; U.S. Pat. No. 5,633,425 to Lonberg et al.; and U.S. Pat. No. 5,661,016 to Lonberg et al., which are hereby incorporated by reference in their entirety.

In certain embodiments, it may be desirable to use an antibody fragment, rather than an intact antibody. Various techniques are known for the production of antibody fragments. Traditionally, these fragments are derived via proteolytic digestion of intact antibodies (e.g. Morimoto et al., “Single-step Purification of F(ab′)2 Fragments of Mouse Monoclonal Antibodies (immunoglobulins GI) by Hydrophobic Interaction High Performance Liquid Chromatography Using TSKgel Phenyl-5PW,” Journal of Biochemical and Biophysical Methods 24:107-117 (1992) and Brennan et al., “Preparation of Bispecific Antibodies by Chemical Recombination of Monoclonal Immunoglobulin G1 Fragments,” Science 229:81-3 (1985), which are hereby incorporated by reference in their entirety). However, these fragments are now typically produced directly by recombinant host cells as described above. Thus Fab, Fv, and scFv antibody fragments can all be expressed in and secreted from E. coli or other host cells, thus allowing the production of large amounts of these fragments. Alternatively, such antibody fragments can be isolated from the antibody phage libraries discussed above. The antibody fragment can also be linear antibodies as described in U.S. Pat. No. 5,641,870 to Rinderknecht et al., which is hereby incorporated by reference, and can be monospecific or bispecific. Other techniques for the production of antibody fragments will be apparent to the skilled practitioner.

The present invention further encompasses variants, derivatives, and equivalents which are substantially homologous to the chimeric, humanized and human antibodies, or antibody fragments thereof. These can contain, for example, conservative substitution mutations, (e.g., the substitution of one or more amino acids by similar amino acids, which maintain or improve the binding activity of the antibody or antibody fragment). Antibodies include members of the various immunoglobulin classes, e.g., IgG, IgM, IgA, IgD, IgE, or subclasses thereof such as IgG1, IgG2, functional Fc, non-functional Fc, etc. In various embodiments of the invention “antibody” refers to an antibody fragment or molecule such as an Fab′, F(ab′)2, scFv (single-chain variable) that retains an antigen binding site and encompasses recombinant molecules comprising one or more variable domains (VH or VL). In some embodiments, the antibody is a camelid antibody or fragment thereof. An antibody can be monovalent, bivalent or multivalent in various embodiments. The antibody may be a chimeric or “humanized” antibody.

For administration to a subject, the agents disclosed herein can be provided in pharmaceutically acceptable compositions. These pharmaceutically acceptable compositions comprise a therapeutically-effective amount of one or more of the agents, formulated together with one or more pharmaceutically acceptable carriers (additives) and/or diluents. The pharmaceutical compositions of the present invention can be specially formulated for administration in solid or liquid form, including those adapted for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intrathecal, intercranially, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, agents can be implanted into a patient or injected using a drug delivery system. (See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is herein incorporated by reference.)

As used herein, the term “pharmaceutically acceptable” refers to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject agent from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The phrase “therapeutically-effective amount” as used herein means that amount of an agent, material, or composition comprising an agent described herein which is effective for producing some desired therapeutic effect in at least a sub-population of cells in an animal at a reasonable benefit/risk ratio applicable to any medical treatment.

The determination of a therapeutically effective amount of the agents and compositions disclosed herein is well within the capability of those skilled in the art. Generally, a therapeutically effective amount can vary with the subject's history, age, condition, sex, and the administration of other pharmaceutically active agents.

As used herein, the term “administer” refers to the placement of an agent or composition into a subject (e.g., a subject in need) by a method or route which results in at least partial localization of the agent or composition at a desired site such that desired effect is produced. Routes of administration suitable for the methods of the invention include both local and systemic routes of administration. Generally, local administration results in more of the administered agents being delivered to a specific location as compared to the entire body of the subject, whereas systemic administration results in delivery of the agents to essentially the entire body of the subject.

The compositions and agents disclosed herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration. Exemplary modes of administration include, but are not limited to, injection, infusion, instillation, inhalation, or ingestion. “Injection” includes, without limitation, intravenous, intramuscular, intraarterial, intrathecal, intraventricular, intracranial, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments of the aspects described herein, the compositions are administered by intravenous infusion or injection.

In some embodiments, the agent decreases the level or activity of the cancer cell gene and the cancer cell gene inhibits MMCCK (e.g., Don't Eat Me (DEM) signal).

In some embodiments, the cancer cell gene is selected from Met, Cd47, Igf1r, Arf1, Notch2, Afdn, Art1, Msn, Slc16a1, Gnai2, Sdc1, Cd4, Cd163, Cftr, Cd8a, Jam2, Icos, Nrg1, Ide, I112rb2, Has2, Gpc1, Insr, Epha2, Jmjd6, and Lrrc4.

In some embodiments, the agent is an antibody or functional fragment or derivative thereof to a cell surface receptor. In some embodiments, the agent increases the level or activity of the cancer cell gene and the cancer cell gene enhances MMCCK (e.g., Eat Me (EM) signal).

In some embodiments, the cancer cell gene is selected from Acvr1b, Acvr2a, Adam9, Adcy1, Atp6ap2, Bmpr2, C5ar2, Cd320, Cd7, Cdc20, Cdh1, Cdh11, Epha4, Fxyd6, Gjb1, Hras, Ifn1r1, I110ra, I113ra1, I121r, Itgav, Itgb1, Itgb3, Lamc2, Lrfn3, Plxnb2, Po1r1c, Psen1, Ptdss1, Pth2r, Ror2, Rtn4r12, Sor11, St14, Stx4a, Tfrc, T1r6, and Tspan1.

In some embodiments, the agent reduces the level or activity of a “Don't Eat Me” signal as described herein or as obtained from a screen described herein. In some embodiments, the agent increases the level or activity of a “Eat Me” signal as described herein or as obtained from a screen described herein. In some embodiments, the agent is identified by an appropriate screen described herein.

Methods of Treating Cancer with Myeloid Cell Checkpoint Inhibitor Enhancers

Some aspects of the present disclosure are directed to a method of treating cancer in a subject comprising administering to the subject a myeloid cell checkpoint inhibitor (MyeICI) or other anti-cancer agent and an agent that modulates the level or activity of a cancer cell gene that modulates myeloid cell-mediated killing of cancer cells in the presence of the myeloid cell checkpoint inhibitor or other anti-cancer agent. Some aspects of the present disclosure are directed to a method of treating cancer in a subject comprising administering to the subject a myeloid cell checkpoint inhibitor (MyeICI) or other anti-cancer agent and an agent that modulates the level or activity of a myeloid cell gene that modulates myeloid cell-mediated killing of cancer cells in the presence of the myeloid cell checkpoint inhibitor or other anti-cancer agent.

The cancer is not limited and may be any cancer disclosed herein. In some embodiments, the cancer is a lung cancer.

The subject is not limited and may be any suitable subject. In some embodiments, the subject is a mammal. In some embodiments, the subject is a rat, mouse, human, or non-human primate. In some embodiments, the subject is a cat, dog, or livestock animal.

The agent is not limited and may be any agent described herein. In some embodiments, the agent is an antibody or functional fragment or functional derivative thereof. In some embodiments, the agent is a small molecule. In some embodiments, the agent is identified by an appropriate screen described herein. In some embodiments, the agent is a polypeptide specifically binding to a cancer cell gene product. In some embodiments, the agent is an RNAi agent, a receptor decoy, or an engineered protein. In some embodiments, the agent is an antibody that binds or blocks a cancer cell gene product. In some embodiments, the agent is an opsonizing antibody (e.g., an antibody that specifically binds to and opsonizes the cancer cell).

The myeloid cell checkpoint inhibitor is not limited and may be any suitable myeloid cell checkpoint inhibitor described herein. The anti-cancer agent is also not limited and may be any suitable anti-cancer agent described herein.

The method of administration is not limited and may be any method disclosed herein.

In some embodiments, the agent decreases the level or activity of the cancer cell gene and the cancer cell gene inhibits myeloid cell-mediated killing of cancer cells in the presence of the myeloid cell checkpoint inhibitor.

In some embodiments, the myeloid cell checkpoint inhibitor is an anti-CD24 antibody that binds, blocks or opsonizes or an agent as described herein that reduces the expression or activity of CD24 and the cancer cell gene is selected from Cd24a, Acvr1b, Acvr2a, Ncstn, Psen1, Itgb1, Tgfbr1, Epha2, Cd320, F2r, Nt5e, and Sdc1.

In some embodiments, the myeloid cell checkpoint inhibitor is an anti-CD47 antibody that binds, blocks or opsonizes or an agent as described herein that reduces the expression or activity of CD47 and the cancer cell gene is selected from Rpsa, Acvr1b, Acvr2a, Ncstn, Alcam, Tmem222, Psen1, Igsf11, Fzd5, Plxnb2, Cadm1, and Lrp5.

In some embodiments, the myeloid cell checkpoint inhibitor is an anti-CD40 agonizing antibody or an agent that increases the expression or activity of CD40 and the cancer cell gene is selected from Rpsa, Cdc20, Mfrp, and Igf1r1.

In some embodiments, the myeloid cell checkpoint inhibitor is an anti-PD-L1 antibody that binds, blocks or opsonizes or an agent as described herein that reduces the expression or activity of PD-L1 and the cancer cell gene is selected from Nectin2 and Ltk.

In some embodiments, the agent increases the level or activity of the cancer cell gene and the cancer cell gene enhances myeloid cell-mediated killing of cancer cells in the presence of the myeloid cell checkpoint inhibitor.

In some embodiments, the myeloid cell checkpoint inhibitor is an anti-CD24 antibody that binds, blocks or opsonizes or an agent as described herein that reduces the expression or activity of CD24 and the cancer cell gene is selected from Efnb3, Pdcd11g2, Hjv, Rnf43, Adam23, Havcr2, Lag3, Erbb2, Art1, Insr, T1r6, Cdh11, T1r2, I117rc, Adora2b, Tfrc, Dnajb11, Ramp3, Igf1r, Arf1, Acvr1, Afdn, Tnfsf13, Ld1r, Atp5b, Atp6ap2, Stx4a, Cdh1, and Cd47.

In some embodiments, the myeloid cell checkpoint inhibitor is an anti-CD47 antibody that binds, blocks or opsonizes or an agent as described herein that reduces the expression or activity of CD47 and the cancer cell gene is selected from Itgb3, Cd99, Retn, Egfr, Atp6ap2, K1rb1a, Adam10, Lamp1, C5ar1, Sstr5, Lrfn3, Sema4b, Igf1r, Ld1r, Fam3c, Met, Erbb2, Cdh11, 1121r, I117rc, Adgrb2, Atp5b, Arf1, Copa, Acvr1, and Stx4a.

In some embodiments, the myeloid cell checkpoint inhibitor is an anti-CD40 agonizing antibody or an agent that increases the expression or activity of CD40 and the cancer cell gene is selected from I118r, I127ra, Ephb2, Adam19, Pdcd1, and Copa.

In some embodiments, the myeloid cell checkpoint inhibitor is an anti-PD-L1 antibody that binds, blocks or opsonizes or an agent as described herein that reduces the expression or activity of PD-L1 and the cancer cell gene is selected from Erbb3, Mp1, Ptprd, Mrc1, Tspan1, Egfr, I117rc, Sdc2, Stx3, Ntrk1, Sstr5, Cdh11, and Copa.

In some embodiments, the subject is a human or a mouse. In some embodiments, the subject is a companion animal (e.g., dog or cat). In some embodiments, the subject is a livestock animal (cow, pig, sheep, goat, chicken, etc.).

### Compositions

Some aspects of the present disclosure are directed to a pharmaceutical composition comprising an agent for the treatment of cancer as described herein and a pharmaceutically acceptable excipient. In some embodiments, the agent modulates a cancer cell gene that modulates MMCCK. In some embodiments, the agent modulates a cancer cell gene that modulates the activity of an MyeICI.

Some aspects of the present disclosure are directed to a pharmaceutical composition comprising a pharmaceutically acceptable excipient and an antibody or functional fragment or derivative thereof, polypeptide, or small molecule specifically binding to and antagonizing Ermp1, Dpm family members (e.g., Dpm1, Dpm2, Dpm3), or Pig family members (e.g., Pigv, Pigk, Piga, Pigc, Pigx, Pigm, Pigl, Pigo, Pigs, Pigf, Pigu, Pigb, Pigh, Pigt, Pigw, Pigq, Pign, PigP). Some aspects of the present disclosure are directed to a pharmaceutical composition comprising a pharmaceutically acceptable excipient and an antibody or functional fragment or derivative thereof, polypeptide, or small molecule specifically binding to Ermp1, Cflar, Slc35a1, Chst2, Copx, Map3k7, Efr3a, Dpm1, Dpm2, Dpm3, or PigP.

Some aspects of the present disclosure are directed to a pharmaceutical composition comprising a pharmaceutically acceptable excipient and a small molecule agent specifically binding to and antagonizing Ermp1, Dpm family members (e.g., Dpm1, Dpm2, Dpm3), or Pig family members (e.g., Pigv, Pigk, Piga, Pigc, Pigx, Pigm, Pigl, Pigo, Pigs, Pigf, Pigu, Pigb, Pigh, Pigt, Pigw, Pigq, Pign, PigP). Some aspects of the present disclosure are directed to a pharmaceutical composition comprising a pharmaceutically acceptable excipient and a small molecule agent specifically binding to and antagonizing Ermp1, Cflar, Slc35a1, Chst2, Copx, Map3k7, Efr3a, Dpm1, Dpm2, Dpm3, or PigP.

Some aspects of the present disclosure are directed to a pharmaceutical composition comprising a pharmaceutically acceptable excipient and an agent that increases the level or activity of Ptdss1, Mtf1, Zbtb14, or Pomp.

Any of the variants described herein (e.g., embodiments, variations, examples, specific examples, figures, etc.) and/or any portion of the variants described herein can be additionally or alternatively combined, aggregated, excluded, used, performed serially, performed in parallel, and/or otherwise applied.

Portions of embodiments of the methods and systems can be embodied and/or implemented at least in part as a machine (e.g., processor) configured to receive a computer-readable medium storing computer-readable instructions. The instructions can be executed by computer-executable components that can be integrated with embodiments of the systems and methods described herein. The computer-readable medium can be stored on any suitable computer-readable media such as RAMs, ROMs, flash memory, EEPROMs, optical devices (CD or DVD), hard drives, floppy drives, or any suitable device. The computer-executable component can be a general or application specific processor, but any suitable dedicated hardware or hardware/firmware combination device can alternatively or additionally execute the instructions.

As a person skilled in the art will recognize from the previous detailed description and from the figures and claims, modifications and changes can be made to embodiments of the methods and systems disclosed herein, and/or variants without departing from the scope defined in the claims. Variants described herein not meant to be restrictive. Certain features included in the drawings may be exaggerated in size, and other features may be omitted for clarity and should not be restrictive. The figures are not necessarily to scale. Section titles herein are used for organizational convenience and are not meant to be restrictive. The description of any variant is not necessarily limited to any section of this specification.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The term “statistically significant” or “significantly” refers to statistical significance and generally means a “p” value greater than 0.05 (calculated by the relevant statistical test). Those skilled in the art will readily appreciate that the relevant statistical test for any particular experiment depends on the type of data being analyzed. Additional definitions are provided in the text of individual sections below.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); The ELISA guidebook (Methods in molecular biology 149) by Crowther J. R. (2000); Immunology by Werner Luttmann, published by Elsevier, 2006. Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Cun-ent Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001) and Davis et al., Basic Methods in Molecular Biology, Elsevier Science Publishing, Inc., New York, USA (1995) which are both incorporated by reference herein in their entireties.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

To systematically investigate myeICs, Applicants have developed a cancer-macrophage co-culture CRISPR screens to identify myeICs in vitro. So far, Applicants have performed multiple genome-wide and cell-surface-gene focused CRISPR screens in two mouse lung cancer cell lines aiming to identify genes that enhance or protect cancer cells from macrophage mediated cytotoxicity.

Genome-wide CRISPR screens were performed in two separate mouse lung cancer cell lines KP-238N1 and KP-368T1. These cell lines are derived from tumors generated in the Kras; p53-driven lung cancer mouse model. Specifically, cancer cells were transduced with a pool of sgRNAs targeting every gene in the genome at low multiplicity of infection (MOI-0.3) to ensure each cancer cell carries a single and unique sgRNA. Transduced cancer cells were then co-cultured with primary mouse bone marrow derived macrophages (BMDMs) at 1:4 ratio or cultured alone for 5 days. All cells were harvested at the end with a minimum coverage of 500×. Genomic DNA from each sample was extracted and sgRNA libraries were amplified by PCR and sequenced by illumina high-seq. Differential enrichment was calculated by comparing the representation of each sgRNA in the co-culture condition relative to the control.

CRISPR screening details are listed below. In the KP-238N1 screen, Applicants have 4 different arms: 1) cancer cell alone, 2) cancer cell co-cultured with macrophages, 3) cancer cell co-cultured with macrophages with anti-CD47 blocking antibody and 4) cancer cell co-cultured with macrophages with anti-CD24 blocking antibody. By comparing 2) vs 1), Applicants uncovered genes that, upon perturbation, enhance or inhibit macrophage mediated cancer cell killing when co-cultured with primary macrophages. In addition, Applicants examined genes that interact with CD47 or CD24 by comparing 3) or 4) to 2). Similar to the set-up of 1) and 2) in KP-238N1, Applicants also performed a parallel screen in KP-368T1 as a biological replicate (Applicants didn't have condition 3 and 4 in this cell line).

### Results

The screens identified many novel putative myeICs by filtering genes with a Discovery Score more than 5 (which integrates the fold-change, p value and number of independent sgRNAs for hit discovery). Encouragingly, the screen identified several known myeICs, such as Cd47 and Qpctl. Importantly, Applicants have uncovered a whole list of undescribed candidates that significantly enhance (e.g., Yars2, Psma6 and Tmem30a) or decrease (e.g., Nlel and Ptdss1) macrophage-mediated cancer cell killing.

Detailed Screen results are listed below:
Genome-wide CRISPR Screens

### Result 0:

By comparing the representation of sgRNAs in cancer cells (using cell line KP-238N1) when co culturing with primary macrophage vs. cancer cells cultured alone, Applicants identified genes that enhance macrophage-mediated cancer cell killing (MMCCK) (what Applicants call “Eat Me” (EM) signals). Knockout of this gene makes cancer cells more resistant to MMCCK, thus the sgRNAs targeting this gene become enriched in the macrophage co-culture vs cancer cell cultured alone. Applicants have also identified genes that inhibit MMCCK (what Applicants call “Don't Eat Me” (DEM) signals). Knockout of this gene makes cancer cells more sensitive to MMCCK, thus the sgRNAs targeting this gene become depleted in the macrophage co-culture vs cancer cell cultured alone.

In this comparison in KP-238N1 cells, Applicants identified 112 putative Don't Eat Me genes and 251 putative Eat Me genes.

TABLE 0
Genes identified as providing EM and DEM signals:

| | average phenotype | | | |
|---|---|---|---|---|
| gene | of strongest 3 | discScore | thres | |
| Met | −1.72288264 | 63.89964653 | TRUE | DEM |
| Cd47 | −1.659760508 | 41.11325513 | TRUE | DEM |
| Igf1r | −1.351999633 | 50.14404153 | TRUE | DEM |
| Arf1 | −0.767788603 | 10.16886901 | TRUE | DEM |
| Notch2 | −0.615208652 | 19.43046911 | TRUE | DEM |
| Afdn | −0.567941253 | 16.9524048 | TRUE | DEM |
| Rpl37a | −0.543063543 | 5.145417826 | TRUE | Control |
| Art1 | −0.465552657 | 6.647458683 | TRUE | DEM |
| Msn | −0.388726245 | 11.45583024 | TRUE | DEM |
| Slc16a1 | −0.34986681 | 8.130899396 | TRUE | DEM |
| Gnai2 | −0.348431429 | 10.1372439 | TRUE | DEM |
| Sdc1 | −0.328646356 | 9.015208209 | TRUE | DEM |
| Cd4 | −0.319230247 | 5.344711998 | TRUE | DEM |
| Cd163 | −0.303400731 | 7.567937946 | TRUE | DEM |
| Cftr | −0.299252676 | 7.309606854 | TRUE | DEM |
| Cd8a | −0.295855403 | 6.727960764 | TRUE | DEM |
| Jam2 | −0.287301314 | 5.661202888 | TRUE | DEM |
| Icos | −0.286122102 | 7.848711157 | TRUE | DEM |
| Nrg1 | −0.275815375 | 5.100179319 | TRUE | DEM |
| Ide | −0.260853676 | 7.155564507 | TRUE | DEM |
| Il12rb2 | −0.251942748 | 5.729356 | TRUE | DEM |
| Has2 | −0.242906678 | 6.058992208 | TRUE | DEM |
| Gpc1 | −0.236306708 | 5.295841489 | TRUE | DEM |
| Insr | −0.226083177 | 5.877068973 | TRUE | DEM |
| Epha2 | −0.2239075 | 5.017964303 | TRUE | DEM |
| Lamc2 | 0.190794672 | 5.130121237 | TRUE | EM |
| C5ar2 | 0.204428205 | 5.423303889 | TRUE | EM |
| St14 | 0.222536204 | 5.359616789 | TRUE | EM |
| Psen1 | 0.225950806 | 6.198132214 | TRUE | EM |
| Sorl1 | 0.227858831 | 7.466436207 | TRUE | EM |
| Ror2 | 0.238514195 | 6.413212307 | TRUE | EM |
| Adcy1 | 0.239104801 | 5.202558048 | TRUE | EM |
| Gjb1 | 0.268038401 | 8.623570812 | TRUE | EM |
| Il13ra1 | 0.272092511 | 5.832549968 | TRUE | EM |
| Fxyd6 | 0.272742435 | 5.043356714 | TRUE | EM |
| Plxnb2 | 0.291674024 | 9.791416845 | TRUE | EM |
| Il21r | 0.311019684 | 5.207246654 | TRUE | EM |
| Cd7 | 0.322806535 | 8.220813146 | TRUE | EM |
| Hras | 0.329128467 | 11.58642534 | TRUE | EM |
| Amfr | 0.410058866 | 14.69086151 | TRUE | EM |
| Acvr2a | 0.415342485 | 15.40455361 | TRUE | EM |
| Ndc80 | 0.419220665 | 5.446342267 | TRUE | Control |
| Stx4a | 0.433119793 | 9.706596081 | TRUE | EM |
| Atp6ap2 | 0.502322383 | 18.63053349 | TRUE | EM |
| Tlr6 | 0.503763876 | 9.090956955 | TRUE | EM |
| Cd320 | 0.543029268 | 11.72774581 | TRUE | EM |
| Acvr1b | 0.550782982 | 20.42787885 | TRUE | EM |
| Rpl15 | 0.560274403 | 5.869521797 | TRUE | Control |
| Itgb3 | 0.612394467 | 22.58315458 | TRUE | EM |
| Cdh1 | 0.689390717 | 25.42253065 | TRUE | EM |
| Itgb1 | 0.917605727 | 34.03289361 | TRUE | EM |
| Tfrc | 0.980833397 | 36.37793193 | TRUE | EM |
| Itgav | 1.254519672 | 46.52862693 | TRUE | EM |
| Ptdss1 | 1.309770996 | 45.5685247 | TRUE | EM |

### Result 1:

By comparing the representation of sgRNA in cancer cells (a different lung cancer cell line KP-368T1) when co-culturing with primary macrophage vs. cancer cells cultured alone, Applicants identified 86 putative Don't Eat Me genes and 245 putative Eat Me genes.

TABLE 1
Genes identified as providing EM and DEM signals (genes
labeled as control are spiked into the reaction):

| | average phenotype | | | |
|---|---|---|---|---|
| gene | of strongest 3 | discScore | thres | |
| Rpl23 | 1.948105761 | 57.57580189 | TRUE | Control |
| Ptdss1 | 1.44975931 | 18.20970232 | TRUE | EM |
| Cdk1 | 0.887444182 | 11.48317343 | TRUE | Control |
| Polr1c | 0.447092967 | 15.20841503 | TRUE | EM |
| Rpl15 | 0.424541103 | 5.276289978 | TRUE | Control |
| Mrpl4 | 0.346254583 | 10.39837686 | TRUE | Control |
| Cdh11 | 0.339927636 | 6.291323835 | TRUE | EM |
| Cdc20 | 0.329385624 | 5.607921002 | TRUE | EM |
| Amfr | 0.31205244 | 10.48692263 | TRUE | Control |
| Lrfn3 | 0.296827202 | 5.951757716 | TRUE | EM |
| Il10ra | 0.296195998 | 7.577276119 | TRUE | EM |
| Acvr1b | 0.271845766 | 5.623343872 | TRUE | EM |
| Tspan1 | 0.270803675 | 6.831703586 | TRUE | EM |
| Il27ra | 0.269029419 | 7.720058961 | TRUE | EM |
| Adam9 | 0.263005263 | 6.916610317 | TRUE | EM |
| Rtn4rl2 | 0.261515112 | 6.143686107 | TRUE | EM |
| Ifnlr1 | 0.257308062 | 6.813270004 | TRUE | EM |
| Epha4 | 0.237660942 | 5.185743862 | TRUE | EM |
| Pth2r | 0.220587222 | 5.487230504 | TRUE | EM |
| Bmpr2 | 0.20257093 | 5.511543427 | TRUE | EM |
| Jmjd6 | −0.214062403 | 6.022271401 | TRUE | DEM |
| Lrrc4 | −0.26769457 | 7.732303045 | TRUE | DEM |
| Pcna | −0.945322263 | 7.090802334 | TRUE | Control |
| Cd47 | −2.398092996 | 32.26236601 | TRUE | DEM |

control = designed positive control genes, not necessarily cell surface genes. Essential genes/Cell cycle genes => to show Cas9 is functional

### Result 2:

By comparing the representation of sgRNA in cancer cells (lung cancer cell line KP-368T1) when co-culturing with primary macrophage with vs. without anti-CD47 blocking antibody treatment, Applicants identified genes that enhance the therapeutic effect of Anti-CD47 treatment and genes that decrease the therapeutic effect of Anti-CD47 treatment.

TABLE 2
Genes identified as providing genes that enhance the therapeutic
effect of Anti-CD47 treatment (synergize) and genes that decrease
the therapeutic effect of Anti-CD24 treatment (inhibit).

| | average phenotype | | | |
|---|---|---|---|---|
| gene | of strongest 3 | discScore | thres | Function |
| Cd47 | 2.268924982 | 68.7384074 | TRUE | I |
| Pcna | 1.067688332 | 39.0806705 | TRUE | Control |
| Rpsa | 0.887126713 | 12.8764747 | TRUE | I |
| Acvr1b | 0.716084405 | 31.1642792 | TRUE | I |
| Acvr2a | 0.532427924 | 23.1714758 | TRUE | I |
| Ncstn | 0.336485748 | 14.2279545 | TRUE | I |
| Alcam | 0.30815688 | 6.36631008 | TRUE | I |
| Tmem222 | 0.275346614 | 8.57142391 | TRUE | I |
| Psen1 | 0.264230855 | 7.41462396 | TRUE | I |
| Igsf11 | 0.262736801 | 6.95998369 | TRUE | I |
| Fzd5 | 0.233333751 | 5.95739383 | TRUE | I |
| Plxnb2 | 0.231883067 | 7.46390472 | TRUE | I |
| Cadm1 | 0.197695538 | 5.31381241 | TRUE | I |
| Lrp5 | 0.179918394 | 5.01294267 | TRUE | I |
| Itgb3 | −0.171228203 | 5.43865479 | TRUE | S |
| Cd99 | −0.17647753 | 5.27365829 | TRUE | S |
| Retn | −0.199687622 | 6.34260024 | TRUE | S |
| Egfr | −0.207053704 | 6.31576053 | TRUE | S |
| Atp6ap2 | −0.216079672 | 8.1574447 | TRUE | S |
| Klrb1a | −0.2207843 | 6.68878932 | TRUE | S |
| Adam10 | −0.221602653 | 7.71231408 | TRUE | S |
| Lamp1 | −0.235127371 | 7.66908372 | TRUE | S |
| C5ar1 | −0.267451156 | 8.26959067 | TRUE | S |
| Sstr5 | −0.271080563 | 10.046374 | TRUE | S |
| Lrfn3 | −0.285051977 | 8.34324713 | TRUE | S |
| Sema4b | −0.289005231 | 12.0084259 | TRUE | S |
| Igf1r | −0.29218579 | 11.3029917 | TRUE | S |
| Ldlr | −0.298341784 | 8.67162779 | TRUE | S |
| Mrpl4 | −0.306819241 | 12.9735367 | TRUE | Control |
| Fam3c | −0.32823947 | 13.242396 | TRUE | S |
| Met | −0.332137255 | 13.8006001 | TRUE | S |
| Erbb2 | −0.336459621 | 14.2268498 | TRUE | S |
| Cdh11 | −0.366954807 | 14.195376 | TRUE | S |
| Il21r | −0.369224166 | 5.62862709 | TRUE | S |
| Il17rc | −0.388052857 | 13.5915471 | TRUE | S |
| Adgrb2 | −0.390429726 | 6.84635627 | TRUE | S |
| Atp5b | −0.402902701 | 14.2918647 | TRUE | S |
| Arf1 | −0.409562254 | 13.0087665 | TRUE | S |
| Polr1c | −0.532421863 | 22.9067231 | TRUE | Control |
| Cdk1 | −0.563943047 | 8.02359646 | TRUE | Control |
| Copa | −0.585344905 | 21.5590775 | TRUE | S |
| Acvr1 | −0.616435051 | 26.674162 | TRUE | S |
| Stx4a | −0.644767793 | 27.1053178 | TRUE | S |
| Rpl23 | −0.717893332 | 10.3167756 | TRUE | Control |

I = inhibit
S = synergize
Control = positive control genes, all are essential genes related to cell cycle, basic cell function

### Result 3:

By comparing the representation of sgRNA in cancer cells (lung cancer cell line KP-368T1) when co-culturing with primary macrophage with vs. without anti-CD24 blocking antibody treatment, Applicants identified genes that enhance the therapeutic effect of Anti-CD24 treatment and genes that decrease the therapeutic effect of Anti-CD24 treatment.

TABLE 3
Genes identified as providing genes that enhance the therapeutic
effect of Anti-CD24 treatment (synergize) and genes that decrease
the therapeutic effect of Anti-CD24 treatment (inhibit).

| | average phenotype | | | |
|---|---|---|---|---|
| gene | of strongest 3 | discScore | thres | function |
| Cd24a | 2.316890583 | 78.7025731 | TRUE | I |
| Acvr1b | 0.988933784 | 33.5931416 | TRUE | I |
| Acvr2a | 0.673187519 | 20.573634 | TRUE | I |
| Ncstn | 0.588268555 | 19.5280763 | TRUE | I |
| Psen1 | 0.471884729 | 15.7554267 | TRUE | I |
| Itgb1 | 0.341346334 | 9.99672251 | TRUE | I |
| Tgfbr1 | 0.299650619 | 6.06070616 | TRUE | I |
| Epha2 | 0.255370915 | 7.43288516 | TRUE | I |
| Cd320 | 0.251772947 | 5.87248841 | TRUE | I |
| F2r | 0.244342366 | 5.01531652 | TRUE | I |
| Nt5e | 0.243642706 | 5.14869585 | TRUE | I |
| Sdc1 | 0.228761481 | 5.52093926 | TRUE | I |
| Efnb3 | −0.206659742 | 5.19210503 | TRUE | S |
| Pdcd1lg2 | −0.23063921 | 5.79456354 | TRUE | S |
| Hjv | −0.258092279 | 5.81456371 | TRUE | S |
| Rnf43 | −0.259474547 | 6.47584812 | TRUE | S |
| Adam23 | −0.268845781 | 6.18470421 | TRUE | S |
| Havcr2 | −0.283496914 | 5.23706619 | TRUE | S |
| Lag3 | −0.28971149 | 6.16654931 | TRUE | S |
| Erbb2 | −0.297227501 | 5.11541735 | TRUE | S |
| Art1 | −0.310884896 | 5.05467534 | TRUE | S |
| Insr | −0.320704192 | 7.1243484 | TRUE | S |
| Tlr6 | −0.323550841 | 9.18631887 | TRUE | S |
| Cdh11 | −0.337631099 | 8.2036328 | TRUE | S |
| Tlr2 | −0.338543769 | 10.0985358 | TRUE | S |
| Il17rc | −0.338743484 | 7.05530093 | TRUE | S |
| Adora 2b | −0.344715685 | 8.31938293 | TRUE | S |
| Tfrc | −0.347854375 | 5.84374755 | TRUE | S |
| Dnajb11 | −0.364928411 | 8.92679192 | TRUE | S |
| Ramp3 | −0.370242805 | 8.69527169 | TRUE | S |
| Ndc80 | −0.372871553 | 9.12109517 | TRUE | Control |
| Igf1r | −0.388099916 | 9.81538696 | TRUE | S |
| Arf1 | −0.404805432 | 11.1371614 | TRUE | S |
| Acvr1 | −0.424999985 | 13.8643281 | TRUE | S |
| Afdn | −0.436069878 | 7.6256412 | TRUE | S |
| Tnfsf13 | −0.443371641 | 7.38811761 | TRUE | S |
| Mrps14 | −0.450395894 | 6.73160641 | TRUE | Control |
| Ldlr | −0.459956286 | 9.79023345 | TRUE | S |
| Mrpl4 | −0.492476441 | 16.3481754 | TRUE | Control |
| Atp5b | −0.589986469 | 7.36394236 | TRUE | S |
| Atp6ap2 | −0.617195047 | 20.9655297 | TRUE | S |
| Stx4a | −0.688716632 | 21.3027089 | TRUE | S |
| Polr1c | −0.733579136 | 12.5243131 | TRUE | Control |
| Cdh1 | −0.745324132 | 25.02896 | TRUE | S |
| Rpl37a | −0.931296361 | 5.01291379 | TRUE | Control |
| Cdk1 | −1.25555684 | 40.00798 | TRUE | Control |
| Cd47 | −2.255848901 | 76.6290451 | TRUE | S |

I = inhibit
S = synergize
Control = positive control genes, all are essential genes related to cell cycle, basic cell function

### Example 4

By comparing the representation of sgRNA in cancer cells (lung cancer cell line KP-368T1) when co-culturing with primary macrophage with vs. without anti-PDL1 blocking antibody treatment, Applicants identified genes that enhance the therapeutic effect of Anti-PDL1 treatment and genes that decrease the therapeutic effect of Anti-PDL1 treatment.

TABLE 4
Genes identified as providing genes that enhance the therapeutic
effect of Anti- PDL1 treatment (synergize) and genes that decrease
the therapeutic effect of Anti-CD24 treatment (inhibit).

| | average phenotype | | | |
|---|---|---|---|---|
| gene | of strongest 3 | discScore | thres | function |
| Pcna | 0.930523446 | 11.2489049 | TRUE | Control |
| Nectin2 | 0.294266358 | 8.26672619 | TRUE | I |
| Ltk | 0.253413009 | 6.61080725 | TRUE | I |
| Erbb3 | −0.20701241 | 5.18045091 | TRUE | S |
| Mpl | −0.216903795 | 5.50424553 | TRUE | S |
| Ptprd | −0.218345131 | 5.01476311 | TRUE | S |
| Mrc1 | −0.239520188 | 6.12049988 | TRUE | S |
| Tspan1 | −0.258669014 | 5.06450557 | TRUE | S |
| Egfr | −0.264209201 | 6.11253826 | TRUE | S |
| Mrpl4 | −0.268705442 | 5.01417514 | TRUE | Control |
| Il17rc | −0.272226542 | 5.98124345 | TRUE | S |
| Sdc2 | −0.275461701 | 7.68737158 | TRUE | S |
| Stx3 | −0.322253663 | 7.3472784 | TRUE | S |
| Ntrk1 | −0.341075127 | 5.00034616 | TRUE | S |
| Sstr5 | −0.360596825 | 6.56624094 | TRUE | S |
| Cdh11 | −0.390224909 | 6.36739656 | TRUE | S |
| Rpl23 | −0.679113481 | 7.21622372 | TRUE | Control |
| Copa | −0.934788784 | 9.74751367 | TRUE | S |
| Rpl37a | −1.020150747 | 10.9770959 | TRUE | Control |

I = inhibit
S = synergize
Control = positive control genes, all are essential genes related to cell cycle, basic cell function

### Example 5

By comparing the representation of sgRNA in cancer cells (lung cancer cell line KP-368T1) when co-culturing with primary macrophage with vs. without anti-CD40 agonizing antibody treatment, Applicants identified genes that enhance the therapeutic effect of anti-CD40 treatment and genes that decrease the therapeutic effect of anti-CD40 treatment.

TABLE 5
Genes identified as enhancing the therapeutic effect
of anti-CD40 treatment (synergize) or decreasing the
therapeutic effect of anti-CD40 treatment (inhibit).

| | average phenotype | | | |
|---|---|---|---|---|
| gene | of strongest 3 | discScore | thres | Function |
| Rpl15 | 0.951944271 | 32.40144569 | TRUE | Control |
| Rpsa | 0.756038964 | 31.15167557 | TRUE | I |
| Cdc20 | 0.491200126 | 8.08610957 | TRUE | I |
| Mfrp | 0.340807318 | 6.305430843 | TRUE | I |
| Igflr1 | 0.210359196 | 5.207733183 | TRUE | I |
| Il18r1 | −0.161249912 | 5.146990774 | TRUE | S |
| Il27ra | −0.19814514 | 6.451166396 | TRUE | S |
| Ephb2 | −0.248997376 | 5.576024486 | TRUE | S |
| Adam19 | −0.265170572 | 7.696499227 | TRUE | S |
| Pdcd1 | −0.315558397 | 5.686542559 | TRUE | S |
| Polr1c | −0.336738481 | 6.39415793 | TRUE | Control |
| Copa | −0.357525388 | 7.024479511 | TRUE | S |

I = inhibit
S = synergize
Control = positive control genes, all are essential genes related to cell cycle, basic cell function

### Example 6

### Cell Surface Molecule-Focused Screen

This is a sub-library targeting all cell surface genes (subset of the genome wide library); several of the hits are consistent with the results from our genome-wide screens, such as Ptdss1.

In summary, from these genome-wide CRISPR screens, Applicants have identified several previously undescribed putative regulators of cancer-macrophage interactions. Applicants are currently investigating the roles of Ermp1, Dpm1 (Dpm2, Dpm3), PigP (all Pig gene family), Cflar, Slc35a1, Chst2, Copx, Map3k7 and Efr3a as potential “Don't Eat Me” signals, as well as Ptdss1, Mtf1, Zbtb14, and Pomp as potential “Eat Me” signals.

### Example 7

Human Cancer Cell Line PC9 Co-Culture Genomic CRISPR Screen with Human Primary Macrophages

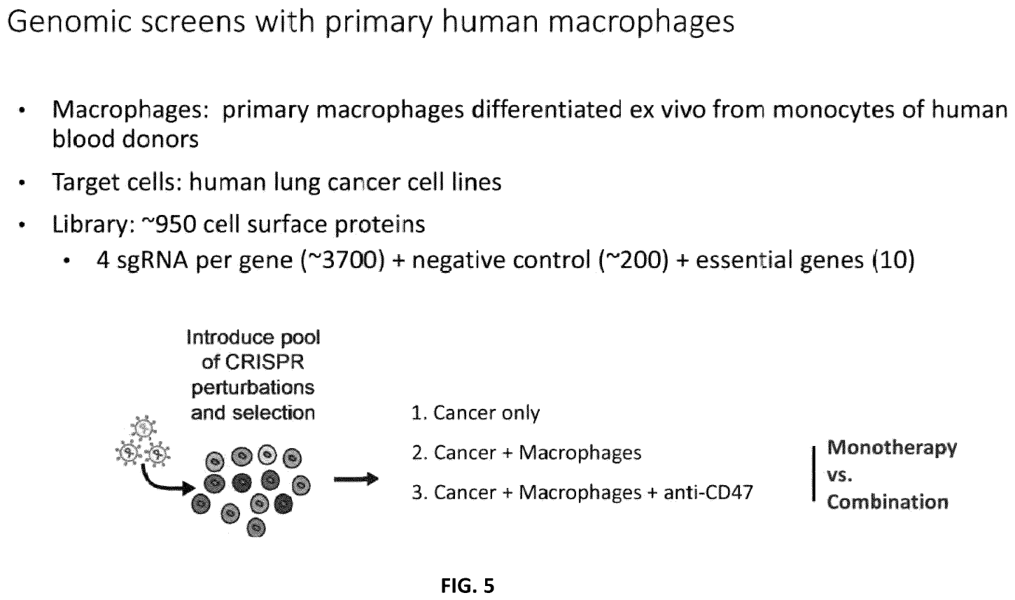

Human primary macrophages differentiated ex vivo from monocytes of human blood donors were co-cultured with a PC9 lung cancer cell line as illustrated in FIG. 5. Results are shown for cancer cell only vs. cancer cell plus macrophages from Donor 97 (Table 6); for cancer cell only vs. cancer cell plus macrophages from mixed donors (Table 7); for cancer cell only vs. cancer cell plus macrophages from Donor 97 plus anti-CD47 (Table 8); for cancer cell only vs. cancer cell plus macrophages from mixed donors plus anti-CD47 (Table 9); for cancer cell only vs. cancer cell plus macrophages from Donor 97 plus anti-CD47 plus IFN-γ (IFN-γ polarizes macrophages to M1-like; Table 10); for cancer cell only vs. cancer cell plus macrophages from mixed donors plus anti-CD47 plus IL-10 (IL-10 polarizes macrophages to M2-like; Table 11). EM=Eat Me, DEM=Don't Eat Me. The genes (e.g., human genes) shown in these tables can be used in the methods described herein in accordance with their characterization as EM or DEM genes. In some particular embodiments of the methods the gene (e.g., the DEM gene) is AXL. In some particular embodiments of the methods the gene (e.g., the EM gene) is one or more genes selected from the group consisting of TNFRSF1A, LTBR, IL6ST, and OSMR.

TABLE 6

| | Mann- | average | | | |
|---|---|---|---|---|---|
| | Whitney | phenotype of | | | |
| Gene | p-value | strongest 3 | discScore | thres | Hits |
| PTDSS1 | 0.000735321 | −0.527820366 | 22.61428357 | TRUE | EM |
| TFRC | 0.00065616 | −0.510483291 | 22.21675631 | TRUE | EM |
| ATP6AP2 | 0.003077349 | −0.613616615 | 21.07414543 | TRUE | EM |
| AXL | 0.003888393 | 0.446282734 | 14.70727945 | TRUE | DEM |
| COPA | 0.015521017 | −0.551678698 | 13.64607469 | TRUE | EM |
| LPAR2 | 0.006117379 | 0.269045368 | 8.142467051 | TRUE | DEM |
| CSF2RB | 0.002875794 | −0.218791276 | 7.602210184 | TRUE | EM |
| STX4 | 0.017938613 | 0.304012368 | 7.258582297 | TRUE | DEM |
| F2RL1 | 0.00521504 | 0.212206526 | 6.623376351 | TRUE | DEM |
| CD5 | 0.005926393 | −0.203531244 | 6.198062626 | TRUE | EM |
| MUSK | 0.006724045 | −0.19502279 | 5.792724027 | TRUE | EM |
| NPY4R | 0.015567425 | −0.230085399 | 5.687209231 | TRUE | EM |
| FAS | 0.00556046 | −0.184388791 | 5.684909249 | TRUE | EM |
| KCND1 | 0.008881661 | −0.202084308 | 5.668512899 | TRUE | EM |
| FPR1 | 0.007856125 | −0.194785315 | 5.605690441 | TRUE | EM |
| CD300LF | 0.006117379 | 0.185149674 | 5.603423445 | TRUE | DEM |
| IGF1R | 0.013873672 | 0.218668322 | 5.554572385 | TRUE | DEM |
| GPR1 | 0.006724045 | 0.185488181 | 5.509519391 | TRUE | DEM |
| KLB | 0.004581745 | −0.168981753 | 5.404158624 | TRUE | EM |
| ERBB4 | 0.011637793 | −0.192187423 | 5.082463681 | TRUE | EM |
| CD79A | 0.017440528 | −0.209109657 | 5.027655756 | TRUE | EM |

TABLE 7

| | Mann- | average | | | |
|---|---|---|---|---|---|
| | Whitney | phenotype of | | | |
| Gene | p-value | strongest 3 | discScore | thres | Hits |
| TFRC | 0.00114914 | −0.6295545 | 21.5479552 | TRUE | EM |
| PTDSS1 | 0.00076362 | −0.5885713 | 21.3615788 | TRUE | EM |
| GIPR | 0.00234127 | 0.3882226 | 11.8907014 | TRUE | DEM |
| STX4 | 0.00651607 | 0.44389764 | 11.2983835 | TRUE | DEM |
| AXL | 0.01844912 | 0.52136531 | 10.5263492 | TRUE | DEM |
| ATP6AP2 | 0.01897229 | −0.4384683 | 8.79066195 | TRUE | EM |
| EPOR | 0.00351959 | 0.30320746 | 8.66179414 | TRUE | DEM |
| SIGLEC1 | 0.0012819 | −0.2369521 | 7.97923144 | TRUE | EM |
| IL6ST | 0.00363883 | 0.26369796 | 7.48869037 | TRUE | DEM |
| HJV | 0.00259598 | −0.2178152 | 6.55762393 | TRUE | EM |
| LILRB4 | 0.00761692 | 0.24975711 | 6.15984131 | TRUE | DEM |
| CD46 | 0.00401892 | −0.2170881 | 6.05596694 | TRUE | EM |
| ADAM8 | 0.00835483 | 0.2350694 | 5.68767972 | TRUE | DEM |
| IL1RL1 | 0.01744053 | 0.27465083 | 5.62327064 | TRUE | DEM |
| CDC20 | 0.01569904 | 0.26289012 | 5.52232214 | TRUE | DEM |
| KCND1 | 0.00401892 | −0.1971878 | 5.50082098 | TRUE | EM |
| ATP5F1B | 0.0160183 | 0.25332761 | 5.29566126 | TRUE | DEM |
| APLNR | 0.00715799 | −0.2070897 | 5.17259628 | TRUE | EM |

TABLE 8

| | Mann- | average | | | |
|---|---|---|---|---|---|
| | Whitney | phenotype of | | | |
| Gene | p-value | strongest 3 | discScore | thres | Hits |
| TFRC | 0.00065616 | −1.453789921 | 51.3249672 | TRUE | EM |
| TNFRSF1A | 0.00065616 | −0.985071577 | 34.77721621 | TRUE | EM |
| CD109 | 0.030066815 | −0.785460672 | 13.25884987 | TRUE | EM |
| LTBR | 0.00065616 | −0.739885321 | 26.12109857 | TRUE | EM |
| NAMPT | 0.037933355 | −0.69268733 | 10.91733229 | TRUE | EM |
| PTDSS1 | 0.000991851 | −0.633432145 | 21.1021739 | TRUE | EM |
| IL6ST | 0.00065616 | −0.531747516 | 18.77294885 | TRUE | EM |
| OSMR | 0.007384257 | −0.511408127 | 12.09160716 | TRUE | EM |
| RPL23A | 0.015521017 | −0.490723532 | 9.846600553 | TRUE | EM |
| ENO1 | 0.00065616 | −0.462875512 | 16.3414742 | TRUE | EM |
| TNFSF10 | 0.008102042 | −0.360595572 | 8.364699447 | TRUE | EM |
| ICAM1 | 0.001029186 | −0.358510262 | 11.87960729 | TRUE | EM |
| ALCAM | 0.002595981 | −0.338437937 | 9.706186248 | TRUE | EM |
| ARF1 | 0.009437972 | −0.32679825 | 7.340446317 | TRUE | EM |
| AMFR | 0.001967105 | −0.318295282 | 9.553827397 | TRUE | EM |
| PVR | 0.003403912 | −0.317294823 | 8.685676853 | TRUE | EM |
| VASN | 0.002341273 | −0.306998795 | 8.957248212 | TRUE | EM |
| IL12RB1 | 0.001480977 | −0.292609067 | 9.182940233 | TRUE | EM |
| CD27 | 0.001191911 | −0.292319798 | 9.479623171 | TRUE | EM |
| ROBO1 | 0.0160183 | −0.278437909 | 5.544690561 | TRUE | EM |
| DYSF | 0.00538525 | −0.273774642 | 6.88937808 | TRUE | EM |
| C3AR1 | 0.002875794 | −0.254998066 | 7.187447168 | TRUE | EM |
| TGFBR1 | 0.007384257 | −0.250850283 | 5.931042 | TRUE | EM |
| FCGR1B | 0.009437972 | −0.249149686 | 5.596327085 | TRUE | EM |
| KLRC1 | 0.002037229 | −0.243187731 | 7.258395278 | TRUE | EM |
| CD81 | 0.009156046 | −0.240441909 | 5.435860202 | TRUE | EM |
| CD101 | 0.004153411 | −0.237807732 | 6.281821673 | TRUE | EM |
| OPRD1 | 0.002508392 | −0.233098512 | 6.723656686 | TRUE | EM |
| IL37 | 0.002109641 | −0.232219633 | 6.891962167 | TRUE | EM |
| GPC4 | 0.006937972 | 0.222350015 | 5.323959359 | TRUE | DEM |
| EFNB2 | 0.007384257 | 0.231307852 | 5.468985587 | TRUE | DEM |
| GFRA3 | 0.007856125 | 0.232272654 | 5.422491612 | TRUE | DEM |
| PYY | 0.00538525 | 0.235480939 | 5.925739531 | TRUE | DEM |
| ACVR2B | 0.010330775 | 0.239919279 | 5.284538048 | TRUE | DEM |
| MARCO | 0.005926393 | 0.240995647 | 5.953357705 | TRUE | DEM |
| PDGFRA | 0.013476536 | 0.260363691 | 5.401466165 | TRUE | DEM |
| ITGAX | 0.001480977 | 0.27190025 | 8.533036157 | TRUE | DEM |
| TNFSF9 | 0.021196863 | 0.279140097 | 5.182017995 | TRUE | DEM |
| CLDN4 | 0.008881661 | 0.282045714 | 6.417767197 | TRUE | DEM |
| STRA6 | 0.014281106 | 0.286548661 | 5.864655645 | TRUE | DEM |
| LDLR | 0.013476536 | 0.291525545 | 6.047945325 | TRUE | DEM |
| MAG | 0.016954611 | 0.29975303 | 5.887125019 | TRUE | DEM |
| FAM3C | 0.001480977 | 0.309820291 | 9.723079469 | TRUE | DEM |
| NECTIN2 | 0.003888393 | 0.317596696 | 8.490360732 | TRUE | DEM |
| F2RL1 | 0.005740798 | 0.322167884 | 8.00794685 | TRUE | DEM |
| LPAR1 | 0.045204454 | 0.351635902 | 5.245038471 | TRUE | DEM |
| GIPR | 0.001769811 | 0.442857752 | 13.51810829 | TRUE | DEM |
| LPAR2 | 0.010330775 | 0.467970723 | 10.30767141 | TRUE | DEM |
| ITGB1 | 0.000991851 | 0.476032004 | 15.85854175 | TRUE | DEM |
| CANX | 0.026335491 | 0.478926147 | 8.390126204 | TRUE | DEM |
| STX4 | 0.004018919 | 0.510529614 | 13.56686959 | TRUE | DEM |
| AXL | 0.001378128 | 0.538483203 | 17.08589688 | TRUE | DEM |
| RPL3 | 0.000920913 | 0.770674882 | 25.94976192 | TRUE | DEM |
| TRAF2 | 0.000735321 | 1.010745948 | 35.12906621 | TRUE | DEM |

TABLE 9

| | Mann- | average | | | |
|---|---|---|---|---|---|
| | Whitney | phenotype of | | | |
| Gene | p-value | strongest 3 | discScore | thres | Hits |
| TFRC | 0.00065616 | −1.5010367 | 52.2173164 | TRUE | EM |
| TNFRSF1A | 0.00065616 | −0.8296231 | 28.8605137 | TRUE | EM |
| LTBR | 0.00065616 | −0.6974783 | 24.2635276 | TRUE | EM |
| NAMPT | 0.03251695 | −0.6752451 | 10.9804524 | TRUE | EM |
| IL6ST | 0.00065616 | −0.63205 | 21.9874404 | TRUE | EM |
| PTDSS1 | 0.00148098 | −0.619819 | 19.1670398 | TRUE | EM |
| OSMR | 0.00538525 | | 12.6864727 | TRUE | EM |
| ENO1 | 0.0017082 | −0.4310197 | 13.036666 | TRUE | EM |
| ALCAM | 0.00082328 | −0.4289161 | 14.4590082 | TRUE | EM |
| ICAM1 | 0.00176981 | −0.4131674 | 12.4272174 | TRUE | EM |
| ADAM19 | 0.02852302 | −0.3340145 | 5.63932006 | TRUE | EM |
| GPR19 | 0.00972762 | −0.3087145 | 6.78846376 | TRUE | EM |
| CD27 | 0.00972762 | −0.2939466 | 6.46372507 | TRUE | EM |
| PVRL3 | 0.01064466 | −0.2854034 | 6.1538259 | TRUE | EM |
| TREM2 | 0.00458175 | −0.2816265 | 7.1992285 | TRUE | EM |
| C3AR1 | 0.00297501 | −0.2740704 | 7.56782766 | TRUE | EM |
| EPHA6 | 0.00972762 | −0.2630631 | 5.78461356 | TRUE | EM |
| ERBB3 | 0.00329171 | −0.2532716 | 6.87190859 | TRUE | EM |
| ACVR1 | 0.00388839 | −0.2457917 | 6.47460882 | TRUE | EM |
| TGFBR3 | 0.00972762 | −0.2311195 | 5.08219216 | TRUE | EM |
| IL21R | 0.00443471 | 0.21589821 | 5.55243732 | TRUE | DEM |
| IL10RB | 0.00592639 | 0.21977864 | 5.34976192 | TRUE | DEM |
| FGFR1 | 0.00738426 | 0.22849035 | 5.32329416 | TRUE | DEM |
| AFDN | 0.00651607 | 0.23380686 | 5.585965 | TRUE | DEM |
| ADAM11 | 0.01096699 | 0.23827336 | 5.10387527 | TRUE | DEM |
| CD151 | 0.01096699 | 0.24866838 | 5.32653937 | TRUE | DEM |
| TNFSF13B | 0.01387367 | 0.24898026 | 5.05537969 | TRUE | DEM |
| MARCO | 0.01234475 | 0.26016751 | 5.42671714 | TRUE | DEM |
| CANX | 0.00785613 | 0.26142676 | 6.01377319 | TRUE | DEM |
| ITGAX | 0.01428111 | 0.26404863 | 5.32505685 | TRUE | DEM |
| ITGAD | 0.00631389 | 0.26480367 | 6.36613631 | TRUE | DEM |
| ITGB2 | 0.00972762 | 0.28544255 | 6.27672663 | TRUE | DEM |
| F11R | 0.00761692 | 0.29032637 | 6.72118154 | TRUE | DEM |
| ADAM17 | 0.00785613 | 0.29367025 | 6.75549166 | TRUE | DEM |
| CD209 | 0.00351959 | 0.30097517 | 8.07060553 | TRUE | DEM |
| JMJD6 | 0.00943797 | 0.30443068 | 6.73794346 | TRUE | DEM |
| ITGB1 | 0.01096699 | 0.30502801 | 6.53377666 | TRUE | DEM |
| CD180 | 0.00340391 | 0.31333308 | 8.45168168 | TRUE | DEM |
| SLAMF7 | 0.00943797 | 0.3163209 | 7.0011089 | TRUE | DEM |
| IL2RB | 0.02495903 | 0.33415885 | 5.85346047 | TRUE | DEM |
| DLL3 | 0.0027796 | 0.34707339 | 9.69556281 | TRUE | DEM |
| NPY2R | 0.00268636 | 0.35503113 | 9.97536342 | TRUE | DEM |
| LPAR2 | 0.00102919 | 0.35507213 | 11.5934656 | TRUE | DEM |
| FAM3C | 0.00088724 | 0.36493716 | 12.1726462 | TRUE | DEM |
| CLDN4 | 0.00307735 | 0.37141383 | 10.1961225 | TRUE | DEM |
| NECTIN2 | 0.00119191 | 0.38389943 | 12.2672302 | TRUE | DEM |
| FCGR2A | 0.03513615 | 0.3953191 | 6.28309344 | TRUE | DEM |
| GIPR | 0.00810204 | 0.41760151 | 9.54526983 | TRUE | DEM |
| KIR2DL3 | 0.06945062 | 0.42495198 | 5.37969647 | TRUE | DEM |
| AXL | 0.00102919 | 0.61076952 | 19.9422451 | TRUE | DEM |
| RPL3 | 0.00943797 | 0.66605262 | 14.7416973 | TRUE | DEM |
| STX4 | 0.05108946 | 0.73237518 | 10.33886 | TRUE | DEM |
| PRTG | 0.08509811 | 0.91487078 | 10.6995115 | TRUE | DEM |
| EGFR | 0.0039048 | 0.99926566 | 26.3025388 | TRUE | DEM |
| TRAF2 | 0.00068162 | 1.14682672 | 39.6880088 | TRUE | DEM |

TABLE 10

| | Mann- | average | | | |
|---|---|---|---|---|---|
| | Whitney | phenotype of | | | |
| Gene | p-value | strongest 3 | discScore | thres | HITS |
| TFRC | 0.00065616 | −1.132859252 | 34.56975095 | TRUE | EM |
| TNFRSF1A | 0.00065616 | −0.870259158 | 26.55638139 | TRUE | EM |
| ATP6AP2 | 0.069450617 | −0.681867084 | 7.572071589 | TRUE | EM |
| PTDSS1 | 0.000991851 | −0.670795122 | 19.31569159 | TRUE | EM |
| IFNGR1 | 0.00065616 | −0.637058473 | 19.44014908 | TRUE | EM |
| IFNGR2 | 0.00065616 | −0.532419596 | 16.24704287 | TRUE | EM |
| TNFRSF1B | 0.000955773 | −0.434995248 | 12.59289056 | TRUE | EM |
| LTBR | 0.018972288 | −0.364781913 | 6.021729253 | TRUE | EM |
| ICAM1 | 0.001107789 | −0.353751278 | 10.02352009 | TRUE | EM |
| ENO1 | 0.023645308 | −0.351323296 | 5.477477103 | TRUE | EM |
| CD27 | 0.002109641 | −0.319669007 | 8.20045124 | TRUE | EM |
| IL6ST | 0.017440528 | −0.298587291 | 5.033660696 | TRUE | EM |
| FAS | 0.005049705 | −0.29665774 | 6.532077782 | TRUE | EM |
| TNFRSF10B | 0.002184407 | −0.272468621 | 6.950113569 | TRUE | EM |
| VASN | 0.004018919 | −0.263395577 | 6.050070805 | TRUE | EM |
| FAM3C | 0.004581745 | 0.224311193 | 5.029912103 | TRUE | DEM |
| ITGA2B | 0.004889124 | 0.240013467 | 5.317127822 | TRUE | DEM |
| GJC2 | 0.006724045 | 0.253289893 | 5.275170594 | TRUE | DEM |
| MC5R | 0.006516066 | 0.254779166 | 5.339516479 | TRUE | DEM |
| CD244 | 0.009437972 | 0.273356425 | 5.307198104 | TRUE | DEM |
| NRXN3 | 0.004434715 | 0.274139207 | 6.184474985 | TRUE | DEM |
| ARF1 | 0.003403912 | 0.27796317 | 6.576899501 | TRUE | DEM |
| ENPP1 | 0.003182885 | 0.281508123 | 6.739467645 | TRUE | DEM |
| SEMA6B | 0.008354827 | 0.297874776 | 5.93440676 | TRUE | DEM |
| PDGFRA | 0.008614645 | 0.300774961 | 5.953834836 | TRUE | DEM |
| KIR2DL1 | 0.01198665 | 0.301606059 | 5.555468791 | TRUE | DEM |
| CD8B | 0.003761729 | 0.305900574 | 7.110622196 | TRUE | DEM |
| IL27RA | 0.008354827 | 0.31334304 | 6.242573063 | TRUE | DEM |
| TNFRSF4 | 0.003638826 | 0.313903519 | 7.340063981 | TRUE | DEM |
| VEGFB | 0.004733177 | 0.317364025 | 7.073544645 | TRUE | DEM |
| GIPR | 0.002109641 | 0.325080699 | 8.339277095 | TRUE | DEM |
| ITGAX | 0.024959031 | 0.335273181 | 5.151759835 | TRUE | DEM |
| EPOR | 0.004291974 | 0.345817471 | 7.848616032 | TRUE | DEM |
| MAG | 0.014281106 | 0.348452765 | 6.164265218 | TRUE | DEM |
| CLEC4G | 0.001149139 | 0.356742831 | 10.05385295 | TRUE | DEM |
| SEMA7A | 0.0160183 | 0.379476631 | 6.531715412 | TRUE | DEM |
| GPR1 | 0.029286165 | 0.458254626 | 6.736426697 | TRUE | DEM |
| ADAM8 | 0.00556046 | 0.465234908 | 10.0573251 | TRUE | DEM |
| RIPK1 | 0.006724045 | 0.503983952 | 10.49627874 | TRUE | DEM |
| ITGB1 | 0.000920913 | 0.51500284 | 14.98873789 | TRUE | DEM |
| LDLR | 0.000955773 | 0.521378487 | 15.09364126 | TRUE | DEM |
| LPAR2 | 0.001281902 | 0.538558824 | 14.93269619 | TRUE | DEM |
| ITGA3 | 0.001191911 | 0.602426963 | 16.88614495 | TRUE | DEM |
| TRAF2 | 0.000707998 | 1.375531339 | 41.53953163 | TRUE | DEM |
| RPL3 | 0.001329211 | 1.407426882 | 38.81155031 | TRUE | DEM |
| EGFR | 0.003219156 | 3.314951918 | 79.20547609 | TRUE | DEM |

TABLE 11

| | Mann- | average | | | |
|---|---|---|---|---|---|
| | Whitney | phenotype of | | | |
| Gene | p-value | strongest 3 | discScore | thres | hits |
| TRAF2 | 0.00068162 | 0.93131521 | 38.6048366 | TRUE | DEM |
| STX4 | 0.00164857 | 0.73995535 | 26.9571154 | TRUE | DEM |
| AXL | 0.0017082 | 0.68648871 | 24.8706042 | TRUE | DEM |
| AFDN | 0.01744053 | 0.49436191 | 11.3800474 | TRUE | DEM |
| CNTN6 | 0.10570497 | 0.41656329 | 5.32181496 | TRUE | DEM |
| LILRB4 | 0.00095577 | 0.41597261 | 16.4434181 | TRUE | DEM |
| LPAR2 | 0.00079293 | 0.41581435 | 16.8787336 | TRUE | DEM |
| IL27RA | 0.03990158 | 0.34752354 | 6.3646783 | TRUE | DEM |
| POLR1C | 0.0019671 | 0.29731226 | 10.5327005 | TRUE | DEM |
| MRPL4 | 0.00176981 | 0.29342566 | 10.5713262 | TRUE | DEM |
| CD180 | 0.00287579 | 0.26273397 | 8.74044879 | TRUE | DEM |
| CD151 | 0.00521504 | 0.24473701 | 7.31354573 | TRUE | DEM |
| GRPR | 0.00504971 | 0.24107594 | 7.24829701 | TRUE | DEM |
| ADORA2B | 0.01950839 | 0.23969955 | 5.36510881 | TRUE | DEM |
| CD33 | 0.01271229 | 0.23314085 | 5.78597962 | TRUE | DEM |
| CANX | 0.00538525 | 0.23071154 | 6.85229158 | TRUE | DEM |
| F2RL1 | 0.00972762 | 0.228466 | 6.01754842 | TRUE | DEM |
| ITGB2 | 0.00415341 | 0.2272597 | 7.08535777 | TRUE | DEM |
| MARCO | 0.00159086 | 0.22467407 | 8.23055795 | TRUE | DEM |
| LRP11 | 0.01308948 | 0.22081605 | 5.4434004 | TRUE | DEM |
| GPR1 | 0.00363883 | 0.22012652 | 7.02849716 | TRUE | DEM |
| FAM3C | 0.00715799 | 0.21691041 | 6.09145967 | TRUE | DEM |
| CD177 | 0.01648062 | 0.21482111 | 5.01425252 | TRUE | DEM |
| CD34 | 0.01198665 | 0.21002002 | 5.2823575 | TRUE | DEM |
| TNFSF11 | 0.01002516 | 0.19883895 | 5.20314438 | TRUE | DEM |
| DDR2 | 0.00672404 | 0.18431688 | 5.24167656 | TRUE | DEM |
| DLL3 | 0.00651607 | 0.17490321 | 5.00520944 | TRUE | DEM |
| CR1 | 0.00538525 | 0.17030715 | 5.05823976 | TRUE | DEM |
| EPHA10 | 0.00504971 | −0.1965473 | 5.909479 | TRUE | EM |
| IFNLR1 | 0.00363883 | −0.2017248 | 6.44094257 | TRUE | EM |
| CD27 | 0.00234127 | −0.2064569 | 7.10963241 | TRUE | EM |
| ERBB2 | 0.01096699 | −0.2110891 | 5.41594101 | TRUE | EM |
| ICAM1 | 0.01129797 | −0.2198667 | 5.60398281 | TRUE | EM |
| HHLA2 | 0.00443471 | −0.2311634 | 7.12093873 | TRUE | EM |
| ERBB4 | 0.00307735 | −0.2436455 | 8.01159182 | TRUE | EM |
| ICAM4 | 0.00176981 | −0.2480851 | 8.93783082 | TRUE | EM |
| NPY4R | 0.01744053 | −0.2597584 | 5.97955301 | TRUE | EM |
| ALCAM | 0.00363883 | −0.2753742 | 8.79251855 | TRUE | EM |
| LTBR | 0.00073532 | −0.2915065 | 11.9578344 | TRUE | EM |
| COPA | 0.01974381 | −0.3224327 | 7.1949053 | TRUE | EM |
| CD101 | 0.00972762 | −0.3523792 | 9.28128953 | TRUE | EM |
| OSMR | 0.00401892 | −0.4201457 | 13.1776616 | TRUE | EM |
| ENO1 | 0.00065616 | −0.4490868 | 18.7127246 | TRUE | EM |
| PCNA | 0.14098063 | −0.4720133 | 5.25743423 | TRUE | EM |
| ATP6AP2 | 0.00268636 | −0.5053889 | 17.0087146 | TRUE | EM |
| CD109 | 0.14668484 | −0.5165324 | 5.63682261 | TRUE | EM |
| IL6ST | 0.00065616 | −0.5896581 | 24.5701035 | TRUE | EM |
| NAMPT | 0.00176981 | −0.6165868 | 22.2139394 | TRUE | EM |
| TNFRSF1A | 0.00065616 | −0.6329289 | 26.3731302 | TRUE | EM |
| PTDSS1 | 0.00065616 | −0.6679979 | 27.8343981 | TRUE | EM |
| TFRC | 0.00065616 | −1.2251479 | 51.0499454 | TRUE | EM |

### Example 8

Human Cancer Cell Line NCI-H358 Co-Culture CRISPR Screen with Human Primary Macrophages

Human primary macrophages differentiated ex vivo from monocytes of human blood donors were co-cultured with an NCI-H358 lung cancer cell line as illustrated in FIG. 5. Results are shown for cancer cell only vs. cancer cell plus macrophages from Donor 97 (Table 12); for cancer cell only vs. cancer cell plus macrophages from mixed donors (Table 13); for cancer cell only vs. cancer cell plus macrophages from Donor 97 plus anti-CD47 (Table 14); and for cancer cell only vs. cancer cell plus macrophages from mixed donors plus anti-CD47 (Table 15). EM=Eat Me, DEM=Don't Eat Me. The genes shown in these tables can be used in the methods described herein in accordance with their characterization as EM or DEM genes. In some particular embodiments of the methods the gene (e.g., the DEM gene) is AXL. In some particular embodiments of the methods the gene (e.g., the EM gene) is one or more genes selected from the group consisting of TNFRSF1A, LTBR, IL6ST, and OSMR.

TABLE 12

| | Mann- | average | | | |
|---|---|---|---|---|---|
| | Whitney | phenotype of | | | |
| Gene | p-value | strongest 3 | discScore | thres | Hits |
| CD72 | 0.01002516 | −0.4175804 | 7.28346916 | TRUE | EM |
| FPR2 | 0.00473318 | −0.3280216 | 6.65429464 | TRUE | EM |
| FCGR1A | 0.03990158 | −0.4658603 | 5.68697967 | TRUE | EM |
| SCARF1 | 0.01002516 | 0.29844116 | 5.20543368 | TRUE | DEM |
| SHANK1 | 0.01950839 | 0.34454295 | 5.14029999 | TRUE | DEM |

TABLE 13

| | Mann- | average | | | |
|---|---|---|---|---|---|
| | Whitney | phenotype of | | | |
| Gene | p-value | strongest 3 | discScore | thres | Hits |
| TNFRSF11B | 0.01347654 | 0.37741207 | 7.80027383 | TRUE | DEM |
| CXCR1 | 0.01844912 | 0.3032164 | 5.80981684 | TRUE | DEM |
| PTH2R | 0.01096699 | 0.28678125 | 6.21072002 | TRUE | DEM |
| CXCR3 | 0.00203723 | 0.27292413 | 8.11528397 | TRUE | DEM |
| MRGPRX2 | 0.01198665 | 0.26600623 | 5.64731505 | TRUE | DEM |
| ICOSLG | 0.01198665 | 0.2643721 | 5.61262241 | TRUE | DEM |
| CD7 | 0.00287579 | −0.2337937 | 6.56497868 | TRUE | EM |
| CNTN4 | 0.00473318 | −0.2463607 | 6.32878584 | TRUE | EM |
| S1PR1 | 0.01428111 | −0.2506004 | 5.10962135 | TRUE | EM |
| PTPRU | 0.00761692 | −0.2518245 | 5.89418526 | TRUE | EM |
| OSMR | 0.03513615 | −0.313889 | 5.0439222 | TRUE | EM |
| SEMA4G | 0.01129797 | −0.3163469 | 6.8058753 | TRUE | EM |
| GPR135 | 0.00242351 | −0.3165534 | 9.14882369 | TRUE | EM |
| DDR1 | 0.02563927 | −0.330831 | 5.81643275 | TRUE | EM |
| IL12RB1 | 0.01064466 | −0.3383717 | 7.37643721 | TRUE | EM |
| EGFR | 0.04409876 | −0.3734552 | 5.59391913 | TRUE | EM |
| RHBDL2 | 0.06631553 | −0.3983882 | 5.18737995 | TRUE | EM |
| CD72 | 0.00132921 | −0.4205546 | 13.3667905 | TRUE | EM |
| SLC16A7 | 0.07106322 | −0.8579795 | 10.8869858 | TRUE | EM |

TABLE 14

| | Mann- | average | | | |
|---|---|---|---|---|---|
| | Whitney | phenotype of | | | |
| gene | p-value | strongest 3 | discScore | thres | Hits |
| HLA-A | 0.00065616 | 1.00267734 | 27.4623459 | TRUE | DEM |
| STX4 | 0.05898511 | 0.53748978 | 5.68531092 | TRUE | DEM |
| SORT1 | 0.00504971 | −0.2662631 | 5.26214401 | TRUE | EM |
| LRP2 | 0.00556046 | −0.2722304 | 5.28205579 | TRUE | EM |
| LRP5 | 0.00458175 | −0.2757906 | 5.55066487 | TRUE | EM |
| CD86 | 0.00340391 | −0.2783747 | 5.9118021 | TRUE | EM |
| ACVRL1 | 0.00363883 | −0.295884 | 6.2098526 | TRUE | EM |
| NCR3 | 0.00761692 | −0.2969311 | 5.4121284 | TRUE | EM |
| TNFSF11 | 0.00861464 | −0.2993145 | 5.31788517 | TRUE | EM |
| CD40 | 0.00429197 | −0.3044127 | 6.20104821 | TRUE | EM |
| BAI2 | 0.00351959 | −0.3085094 | 6.51324074 | TRUE | EM |
| FZD1 | 0.00351959 | −0.3085832 | 6.51479711 | TRUE | EM |
| ADAM29 | 0.01064466 | −0.3087029 | 5.24058697 | TRUE | EM |
| FCGR2A | 0.00835483 | −0.3182665 | 5.69102738 | TRUE | EM |
| IL17RB | 0.01387367 | −0.3192562 | 5.10365602 | TRUE | EM |
| SLC40A1 | 0.0027796 | −0.3216698 | 7.07481594 | TRUE | EM |
| ANXA2 | 0.00972762 | −0.3234694 | 5.60016562 | TRUE | EM |
| TGFBR2 | 0.01234475 | −0.3239228 | 5.31959891 | TRUE | EM |
| GPIHBP1 | 0.00307735 | −0.3255329 | 7.03598747 | TRUE | EM |
| PITPNM3 | 0.00672404 | −0.3262692 | 6.09889051 | TRUE | EM |
| EFNB3 | 0.01512776 | −0.3275281 | 5.12997055 | TRUE | EM |
| HTR6 | 0.01163779 | −0.334624 | 5.56908349 | TRUE | EM |
| TLR5 | 0.01897229 | −0.3389969 | 5.02272997 | TRUE | EM |
| LRP6 | 0.01793861 | −0.3403786 | 5.11446321 | TRUE | EM |
| TFRC | 0.00268636 | −0.3404632 | 7.53157152 | TRUE | EM |
| ACVR1C | 0.01096699 | −0.3423796 | 5.7741184 | TRUE | EM |
| XCR1 | 0.00210964 | −0.3450109 | 7.94376169 | TRUE | EM |
| TNFRSF12A | 0.00376173 | −0.3460202 | 7.21913139 | TRUE | EM |
| F2RL1 | 0.01096699 | −0.3461878 | 5.83834281 | TRUE | EM |
| PROKR2 | 0.01387367 | −0.3494581 | 5.58646555 | TRUE | EM |
| GP1BA | 0.01033078 | −0.3533117 | 6.03739134 | TRUE | EM |
| CD3G | 0.00203723 | −0.3537122 | 8.19027433 | TRUE | EM |
| FPR2 | 0.00297501 | −0.3538181 | 7.69205387 | TRUE | EM |
| ITGB6 | 0.00307735 | −0.3559105 | 7.69256243 | TRUE | EM |
| IL20RB | 0.00401892 | −0.3563899 | 7.34739795 | TRUE | EM |
| SEMA6A | 0.00488912 | −0.3617179 | 7.19229802 | TRUE | EM |
| PECAM1 | 0.0027796 | −0.3635879 | 7.99676504 | TRUE | EM |
| GPR35 | 0.00203723 | −0.3678028 | 8.51654489 | TRUE | EM |
| CD72 | 0.00972762 | −0.3686332 | 6.38207882 | TRUE | EM |
| CD93 | 0.00148098 | −0.3690034 | 8.98408199 | TRUE | EM |
| ADIPOR2 | 0.00164857 | −0.3690312 | 8.83691436 | TRUE | EM |
| THBD | 0.02364531 | −0.3721204 | 5.20730799 | TRUE | EM |
| ACKR2 | 0.02704799 | −0.3748841 | 5.05762741 | TRUE | EM |
| NRXN1 | 0.00651607 | −0.3754041 | 7.0614383 | TRUE | EM |
| GNRHR | 0.00568773 | −0.3759693 | 7.26309464 | TRUE | EM |
| CAV1 | 0.00915605 | −0.3761644 | 6.59758753 | TRUE | EM |
| LRP11 | 0.02239201 | −0.3810231 | 5.40943486 | TRUE | EM |
| TGFBR3 | 0.00388839 | −0.3818557 | 7.91951992 | TRUE | EM |
| ITGA9 | 0.01129797 | −0.3845629 | 6.44279574 | TRUE | EM |
| PTCH2 | 0.00611738 | −0.3852348 | 7.3372492 | TRUE | EM |
| PLXNB2 | 0.00183346 | −0.3885581 | 9.1501645 | TRUE | EM |
| CD7 | 0.02364531 | −0.3895611 | 5.45136544 | TRUE | EM |
| SDC2 | 0.0027796 | −0.3908489 | 8.59634474 | TRUE | EM |
| TNFSF15 | 0.00401892 | −0.3924191 | 8.09018033 | TRUE | EM |
| IL22RA1 | 0.00363883 | −0.4001121 | 8.3973348 | TRUE | EM |
| HAVCR2 | 0.01033078 | −0.4028976 | 6.88471505 | TRUE | EM |
| LMBR1L | 0.00153501 | −0.4078393 | 9.87499879 | TRUE | EM |
| IFNLR1 | 0.01271229 | −0.4094011 | 6.67847418 | TRUE | EM |
| PDGFRA | 0.00415341 | −0.4116614 | 8.43624567 | TRUE | EM |
| ULBP2 | 0.02633549 | −0.4149744 | 5.63989039 | TRUE | EM |
| FGFR4 | 0.00297501 | −0.4169418 | 9.06437219 | TRUE | EM |
| GFRA4 | 0.01096699 | −0.4169788 | 7.03220851 | TRUE | EM |
| PTPRG | 0.00611738 | −0.4194838 | 7.98956201 | TRUE | EM |
| KLB | 0.02777706 | −0.422439 | 5.65720975 | TRUE | EM |
| VASN | 0.04091775 | −0.4233568 | 5.05667136 | TRUE | EM |
| APLP2 | 0.0333708 | −0.4281451 | 5.44007394 | TRUE | EM |
| CXCR1 | 0.03006682 | −0.4366321 | 5.71803075 | TRUE | EM |
| MMP24 | 0.00488912 | −0.4443698 | 8.83572369 | TRUE | EM |
| AXL | 0.04301599 | −0.4470455 | 5.2560712 | TRUE | EM |
| CD3D | 0.03086528 | −0.4582906 | 5.95677655 | TRUE | EM |
| IL9R | 0.01744053 | −0.4608045 | 6.97245087 | TRUE | EM |
| TMEM222 | 0.00242351 | −0.4648309 | 10.4616427 | TRUE | EM |
| TIE1 | 0.05108946 | −0.4972766 | 5.52700994 | TRUE | EM |
| FGFR3 | 0.0014287 | −0.5041196 | 12.341445 | TRUE | EM |
| NECTIN4 | 0.00458175 | −0.5232965 | 10.5320624 | TRUE | EM |
| TRHR | 0.00401892 | −0.5486814 | 11.3117129 | TRUE | EM |
| ENPEP | 0.00473318 | −0.5569049 | 11.1408043 | TRUE | EM |
| EPHA2 | 0.02364531 | −0.5970876 | 8.35541136 | TRUE | EM |
| CD8B | 0.00164857 | −0.6510728 | 15.5907538 | TRUE | EM |
| HHLA2 | 0.00234127 | −0.6564801 | 14.8596619 | TRUE | EM |
| ADAM19 | 0.02563927 | −0.8013781 | 10.9717144 | TRUE | EM |
| FCGR1A | 0.08509811 | −0.8986657 | 8.27476321 | TRUE | EM |

TABLE 15

| | Mann- | average | | | |
|---|---|---|---|---|---|
| | Whitney | phenotype of | | | |
| gene | p-value | strongest 3 | discScore | thres | Hits |
| HLA-A | 0.00065616 | 1.58169914 | 45.7784631 | TRUE | DEM |
| STX4 | 0.0160183 | 0.4982518 | 8.13407419 | TRUE | DEM |
| CCR4 | 0.03168189 | 0.43708124 | 5.95827489 | TRUE | DEM |
| APP | 0.01387367 | 0.35455574 | 5.98945749 | TRUE | DEM |
| NECTIN2 | 0.01387367 | 0.32214592 | 5.44196331 | TRUE | DEM |
| GAS1 | 0.00458175 | −0.2606198 | 5.54286009 | TRUE | EM |
| CALCRL | 0.00429197 | −0.2673643 | 5.7552811 | TRUE | EM |
| MC3R | 0.00401892 | −0.2762161 | 6.01752554 | TRUE | EM |
| FAP | 0.00388839 | −0.2791092 | 6.1169463 | TRUE | EM |
| PLXNA4 | 0.00738426 | −0.2887959 | 5.59780393 | TRUE | EM |
| HHLA2 | 0.00631389 | −0.2932504 | 5.86549552 | TRUE | EM |
| TFRC | 0.00183346 | −0.2940247 | 7.31673972 | TRUE | EM |
| GPR151 | 0.00861464 | −0.2941058 | 5.52173782 | TRUE | EM |
| EPHB4 | 0.01198665 | −0.2948537 | 5.15114999 | TRUE | EM |
| MUSK | 0.00761692 | −0.2960559 | 5.70225934 | TRUE | EM |
| IGF2R | 0.00538525 | −0.3006138 | 6.20163226 | TRUE | EM |
| GP6 | 0.00234127 | −0.3124306 | 7.47311629 | TRUE | EM |
| SLAMF8 | 0.00415341 | −0.3128491 | 6.77493121 | TRUE | EM |
| CX3CR1 | 0.01271229 | −0.3145543 | 5.4223151 | TRUE | EM |
| ADAM29 | 0.01744053 | −0.3169735 | 5.06818703 | TRUE | EM |
| EPHA10 | 0.01428111 | −0.317096 | 5.32041102 | TRUE | EM |
| TNFRSF13B | 0.00835483 | −0.3193164 | 6.03367407 | TRUE | EM |
| IFNAR1 | 0.00443471 | −0.323283 | 6.91722097 | TRUE | EM |
| PDGFRA | 0.00259598 | −0.325485 | 7.65263215 | TRUE | EM |
| EFNB3 | 0.00164857 | −0.3264209 | 8.25993196 | TRUE | EM |
| ADIPOR2 | 0.00159086 | −0.3264557 | 8.30675002 | TRUE | EM |
| NPSR1 | 0.00835483 | −0.3269187 | 6.17732383 | TRUE | EM |
| SDC4 | 0.00458175 | −0.3284174 | 6.9847795 | TRUE | EM |
| TIE1 | 0.00738426 | −0.3323835 | 6.44267375 | TRUE | EM |
| CDH1 | 0.01950839 | −0.3323943 | 5.1676778 | TRUE | EM |
| LMBR1L | 0.00429197 | −0.3324742 | 7.15683681 | TRUE | EM |
| FZD10 | 0.0160183 | −0.3325988 | 5.42975115 | TRUE | EM |
| AGTR1 | 0.00242351 | −0.3326615 | 7.91167362 | TRUE | EM |
| TRHR | 0.01064466 | −0.3336544 | 5.98545173 | TRUE | EM |
| GNRHR | 0.00817464 | −0.3366583 | 6.39034489 | TRUE | EM |
| FCGR2B | 0.01387367 | −0.3366729 | 5.68736462 | TRUE | EM |
| ROBO2 | 0.00861464 | −0.3402954 | 6.38893191 | TRUE | EM |
| SELP | 0.00888166 | −0.3415111 | 6.3705893 | TRUE | EM |
| OPRK1 | 0.00307735 | −0.346834 | 7.92159742 | TRUE | EM |
| ROBO4 | 0.01950839 | −0.347059 | 5.39566777 | TRUE | EM |
| NCAM1 | 0.00473318 | −0.3475973 | 7.34806236 | TRUE | EM |
| CANX | 0.01744053 | −0.3490848 | 5.58162455 | TRUE | EM |
| NTRK1 | 0.01198665 | −0.3508741 | 6.12983723 | TRUE | EM |
| ACKR1 | 0.01897229 | −0.3519678 | 5.51071462 | TRUE | EM |
| EPCAM | 0.00183346 | −0.3529145 | 8.78219965 | TRUE | EM |
| GJC3 | 0.00631389 | −0.3577947 | 7.15648798 | TRUE | EM |
| TNFRSF12A | 0.01347654 | −0.3599898 | 6.12254185 | TRUE | EM |
| CD79B | 0.00203723 | −0.3609088 | 8.83093542 | TRUE | EM |
| IL20RB | 0.00340391 | −0.3638805 | 8.16600704 | TRUE | EM |
| HAVCR1 | 0.00810204 | −0.3642231 | 6.92640214 | TRUE | EM |
| GPR75 | 0.00242351 | −0.3663588 | 8.71309283 | TRUE | EM |
| CHRNA7 | 0.01002516 | −0.3676251 | 6.68190244 | TRUE | EM |
| NECTIN4 | 0.00123615 | −0.3735001 | 9.87589561 | TRUE | EM |
| TNFRSF9 | 0.00672404 | −0.3830489 | 7.56641018 | TRUE | EM |
| LHCGR | 0.00176981 | −0.3842204 | 9.61484629 | TRUE | EM |
| IL9R | 0.01469906 | −0.3866084 | 6.4426882 | TRUE | EM |
| FZD6 | 0.03086528 | −0.3884747 | 5.33573242 | TRUE | EM |
| IL12RB1 | 0.01950839 | −0.3899907 | 6.06311926 | TRUE | EM |
| GPR35 | 0.01271229 | −0.3917904 | 6.75371679 | TRUE | EM |
| ITGB4 | 0.03604833 | −0.4060547 | 5.32828535 | TRUE | EM |
| TMEM222 | 0.01695461 | −0.4099604 | 6.60072881 | TRUE | EM |
| LRP5 | 0.01198665 | −0.4114899 | 7.18880716 | TRUE | EM |
| UTS2R | 0.02495903 | −0.4142401 | 6.03706706 | TRUE | EM |
| NPBWR1 | 0.0008547 | −0.4180557 | 11.6631991 | TRUE | EM |
| CD93 | 0.00672404 | −0.4463292 | 8.81639439 | TRUE | EM |
| ADORA1 | 0.04633343 | −0.451827 | 5.4810576 | TRUE | EM |
| PLXNB2 | 0.00119191 | −0.4521923 | 12.0217122 | TRUE | EM |
| NRXN2 | 0.01428111 | −0.4526406 | 7.59465275 | TRUE | EM |
| GFRA4 | 0.00234127 | −0.4536495 | 10.8509726 | TRUE | EM |
| FPR2 | 0.03168189 | −0.4545872 | 6.19691523 | TRUE | EM |
| ART1 | 0.03793336 | −0.4598267 | 5.94133149 | TRUE | EM |
| AXL | 0.00715799 | −0.4711894 | 9.19109037 | TRUE | EM |
| SSTR4 | 0.00376173 | −0.4721364 | 10.4090683 | TRUE | EM |
| ERBB4 | 0.06479226 | −0.4751191 | 5.13446521 | TRUE | EM |
| IL6R | 0.00340391 | −0.4778627 | 10.723934 | TRUE | EM |
| CD84 | 0.03604833 | −0.5076363 | 6.66124802 | TRUE | EM |
| TLR9 | 0.01064466 | −0.5397081 | 9.68186583 | TRUE | EM |
| DCC | 0.02364531 | −0.5420151 | 8.01497247 | TRUE | EM |
| ENPEP | 0.0019671 | −0.546108 | 13.4380427 | TRUE | EM |
| EPHA2 | 0.01428111 | −0.5580714 | 9.36362982 | TRUE | EM |
| CLEC4G | 0.00376173 | −0.5761521 | 12.7022739 | TRUE | EM |
| IL11RA | 0.07270639 | −0.5991756 | 6.20242409 | TRUE | EM |
| FGFR3 | 0.01950839 | −0.6018733 | 9.35722249 | TRUE | EM |
| PVR | 0.00210964 | −0.6080361 | 14.7939327 | TRUE | EM |
| JAG1 | 0.00110779 | −0.6080976 | 16.3422761 | TRUE | EM |
| CD8B | 0.00065616 | −0.7318988 | 21.1830439 | TRUE | EM |
| FCGR1A | 0.05898511 | −0.794692 | 8.88267489 | TRUE | EM |

### BIBLIOGRAPHY

1. Reck, M. et al. Pembrolizumab versus Chemotherapy for PD-L1-Positive Non-Small-Cell Lung Cancer. N. Engl. J. Med. 375, 1823-1833 (2016).
2. Hodi, F. S. et al. Improved survival with ipilimumab in patients with metastatic melanoma. N. Engl. J. Med. 363, 711-723 (2010).
3. Pardoll, D. M. The blockade of immune checkpoints in cancer immunotherapy. Nature Reviews Cancer vol. 12 252-264 (2012).
4. Ribas, A. & Wolchok, J. D. Cancer immunotherapy using checkpoint blockade. Science vol. 359 1350-1355 (2018).
5. Korman, A. J., Peggs, K. S. & Allison, J. P. Checkpoint Blockade in Cancer Immunotherapy. Advances in Immunology 297-339 (2006) doi:10.1016/s0065-2776(06)90008-x.
6. Freeman, G. J. et al. Engagement of the PD-1 immunoinhibitory receptor by a novel B7 family member leads to negative regulation of lymphocyte activation. J. Exp. Med. 192, 1027-1034 (2000).
7. Chen, D. S. & Mellman, I. Elements of cancer immunity and the cancer-immune set point. Nature vol. 541 321-330 (2017).
8. Forte, C. I. B., Almodovar, T., Duarte, I. & Da Costa, J. D. Immune-Related Adverse Events Associated with Immune Checkpoint Blockade in Lung Cancer—A restrospective study. Lung cancer (2019) doi:10.1183/13993003.congress-2019.pa372.
9. Chan, T. A., Wolchok, J. D. & Snyder, A. Genetic Basis for Clinical Response to CTLA-4 Blockade in Melanoma. The New England journal of medicine vol. 373 1984 (2015).
10. Brahmer, J. R. et al. Safety and activity of anti-PD-L1 antibody in patients with advanced cancer. N. Engl. J. Med. 366, 2455-2465 (2012).
11. Marin-Acevedo, J. A., Kimbrough, E. O. & Lou, Y. Next generation of immune checkpoint inhibitors and beyond. J. Hematol. Oncol. 14, 1-29 (2021).
12. Binnewies, M. et al. Understanding the tumor immune microenvironment (TIME) for effective therapy. Nat. Med. 24, 541-550 (2018).
13. Lavin, Y. et al. Innate Immune Landscape in Early Lung Adenocarcinoma by Paired Single-Cell Analyses. Cell 169, 750-765.e17 (2017).
14. Mantovani, A., Marchesi, F., Malesci, A., Laghi, L. & Allavena, P. Tumour-associated macrophages as treatment targets in oncology. Nature Reviews Clinical Oncology vol. 14 399-416 (2017).
15. Gentles, A. J. et al. The prognostic landscape of genes and infiltrating immune cells across human cancers. Nat. Med. 21, 938-945 (2015).
16. Mantovani, A., Marchesi, F., Jaillon, S., Garlanda, C. & Allavena, P. Tumor-associated myeloid cells: diversity and therapeutic targeting. Cell. Mol. Immunol. 18, 566-578 (2021).
17. Binnewies, M. et al. Targeting TREM2 on tumor-associated macrophages enhances immunotherapy. Cell Rep. 37, 109844 (2021).
18. Jahchan, N. S. et al. Tuning the Tumor Myeloid Microenvironment to Fight Cancer. Front. Immunol. 10, 1611 (2019).
19. Weiskopf, K. Targeting the CD47-SIRP Alpha Interaction to Stimulate Macrophage Destruction of Cancer. (2016).
20. DeNardo, D. G. & Ruffell, B. Macrophages as regulators of tumour immunity and immunotherapy. Nat. Rev. Immunol. 19, 369-382 (2019).
21. Pyonteck, S. M. et al. CSF-1R inhibition alters macrophage polarization and blocks glioma progression. Nat. Med. 19, 1264-1272 (2013).
22. Noel, M. S. et al. Orally administered CCR2 selective inhibitor CCX872-b clinical trial in pancreatic cancer. Journal of Clinical Oncology vol. 35 276-276 (2017).
23. Willingham, S. B. et al. The CD47-signal regulatory protein alpha (SIRPa) interaction is a therapeutic target for human solid tumors. Proc. Natl. Acad. Sci. U.S.A. 109, 6662-6667 (2012).
24. Advani, R. et al. CD47 Blockade by Hu5F9-G4 and Rituximab in Non-Hodgkin's Lymphoma. New England Journal of Medicine vol. 379 1711-1721 (2018).
25. Volkmer, A. K. et al. CD47 antibody blockade as novel therapy for ovarian and breast cancer. Geburtshilfe und Frauenheilkunde vol. 74 (2014).
26. Kosaka, A. et al. CD47 blockade enhances the efficacy of intratumoral STING-targeting therapy by activating phagocytes. J. Exp. Med. 218, (2021).
27. Upton, R. et al. Combining CD47 blockade with trastuzumab eliminates HER2-positive breast cancer cells and overcomes trastuzumab tolerance. Proc. Natl. Acad. Sci. U.S.A 118, (2021).
28. Gholamin, S. et al. Disrupting the CD47-SIRPa anti-phagocytic axis by a humanized anti-CD47 antibody is an efficacious treatment for malignant pediatric brain tumors. Science Translational Medicine vol. 9 eaaf2968 (2017).
29. Chao, M. P. et al. Calreticulin is the dominant pro-phagocytic signal on multiple human cancers and is counterbalanced by CD47. Sci. Transl. Med. 2, 63ra94 (2010).
30. Liu, X. et al. CD47 blockade triggers T cell-mediated destruction of immunogenic tumors. Nat. Med. 21, (2015).
31. Tseng, D. et al. Anti-CD47 antibody-mediated phagocytosis of cancer by macrophages primes an effective antitumor T-cell response. Proc. Natl. Acad. Sci. U.S.A 110, 11103-11108 (2013).
32. Vonderheide, R. H. CD47 blockade as another immune checkpoint therapy for cancer. Nature Medicine vol. 21 1122-1123 (2015).
33. Oldenborg, P. A. et al. Role of CD47 as a marker of self on red blood cells. Science 288, 2051-2054 (2000).
34. Oldenborg, P.-A. Role of CD47 in erythroid cells and in autoimmunity. Leuk. Lymphoma 45, 1319-1327 (2004).
35. Sikic, B. I. et al. First-in-Human, First-in-Class Phase I Trial of the Anti-CD47 Antibody Hu5F9-G4 in Patients With Advanced Cancers. J. Clin. Oncol. 37, 946-953 (2019).
36. Gordon, S. R. et al. PD-1 expression by tumour-associated macrophages inhibits phagocytosis and tumour immunity. Nature 545, 495-499 (2017).
37. Barkal, A. A. et al. Engagement of MHC class I by the inhibitory receptor LILRB1 suppresses macrophages and is a target of cancer immunotherapy. Nature Immunology vol. 19 76-84 (2018).
38. Barkal, A. A. et al. CD24 signalling through macrophage Siglec-10 is a target for cancer immunotherapy. Nature 572, 392-396 (2019).
39. Kamber, R. A. et al. Inter-cellular CRISPR screens reveal regulators of cancer cell phagocytosis. Nature 597, 549-554 (2021).
40. Logtenberg, M. E. W. et al. Glutaminyl cyclase is an enzymatic modifier of the CD47-SIRPα axis and a target for cancer immunotherapy. Nat. Med. 25, 612-619 (2019).
41. Weiskopf, K. et al. CD47-blocking immunotherapies stimulate macrophage-mediated destruction of small-cell lung cancer. J. Clin. Invest. 126, 2610-2620 (2016).
42. Engblom, C., Pfirschke, C. & Pittet, M. J. The role of myeloid cells in cancer therapies. Nat. Rev. Cancer 16, 447-462 (2016).
43. Horlbeck, M. A. et al. Compact and highly active next-generation libraries for CRISPR-mediated gene repression and activation. Elife 5, (2016).
44. Mair, B. et al. High-throughput genome-wide phenotypic screening via immunomagnetic cell sorting. Nature Biomedical Engineering 3, 796-805 (2019).

What is claimed is:

1. A method of screening for a modulator of myeloid cell-mediated killing of cancer cells, comprising
a) providing a population of cancer cells expressing a targetable endonuclease and an sgRNA library targeting genes,
b) contacting the cancer cells with myeloid cells capable of having an anticancer response,
c) coculturing the cancer cells and the myeloid cells, and
d) measuring the relative abundance of each sgRNA of the sgRNA library in the cocultured cancer cells as compared to the abundance of each gRNA in control cancer cells not contacted with the myeloid cells,
wherein the differential relative abundance of an sgRNA as compared to the control indicates that the gene targeted by the sgRNA is a candidate modulator of myeloid cell-mediated killing of cancer cells.

2. The method of claim 1, wherein the population of cancer cells of step a) has been transduced with a pool of sgRNA targeting every gene expressed on the cell surface in the genome of the cancer cell at a multiplicity of infection of about 0.2 to 0.4.

3. The method of claim 1, wherein the population of cancer cells of step a) has been transduced with a pool of sgRNA targeting every gene in the genome of the cancer cell at a multiplicity of infection of about 0.2 to 0.4.

4. The method of claims 1-3, wherein at least two sgRNA target each gene of the targeted genes.

5. The method of claims 1-4, wherein the cancer cells are a cancer cell line.

6. The method of claims 1-5, wherein the myeloid cells are macrophages, preferably polarized macrophages, M1 macrophages, M2 macrophages, resident macrophages, or tumor-associated macrophages.

7. The method of claims 1-6, wherein the population of cancer cells express the targetable endonuclease.

8. The method of claims 1-7, wherein the cancer cells and myeloid cells are cocultured for 1 day or more in step c).

9. The method of claims 1-8, wherein an increased abundance of sgRNA targeting a gene as compared to the control indicates that the product of the gene enhances myeloid cell-mediated killing of the cancer cells.

10. The method of claims 1-8, wherein a decreased abundance of sgRNA targeting a gene as compared to the control indicates that the product of the gene inhibits myeloid cell-mediated killing of the cancer cells.

11. A method of screening for a modulator of myeloid cell-mediated killing of cancer cells, comprising
a) providing a population of cancer cells expressing an RNAi library,
b) contacting the cancer cells with myeloid cells capable of having an anticancer response,
c) coculturing the cancer cells and the myeloid cells, and
d) measuring the relative abundance of each RNAi agent of the RNAi library in the cocultured cancer cells as compared to the abundance of each RNAi agent in control cancer cells not contacted with the myeloid cells,
wherein the differential relative abundance of an RNAi agent as compared to the control indicates that the gene targeted by the RNAi is a candidate modulator of myeloid cell-mediated killing of cancer cells.

12. A method of screening for a modulator of myeloid cell-mediated killing of cancer cells, comprising
a) providing a population of myeloid cells capable of having an anticancer response and expressing a targetable endonuclease and an sgRNA library targeting genes,
b) contacting cancer cells with the myeloid cells,
c) coculturing the cancer cells and the myeloid cells, and
d) identifying targeted genes in the myeloid cells that enhance or inhibit myeloid cell-mediated killing of cancer cells.

13. A method of screening for a modulator of myeloid cell-mediated killing of cancer cells, comprising
a) providing a population of myeloid cells capable of having an anticancer response and expressing an RNAi library,
b) contacting cancer cells with the myeloid cells,
c) coculturing the cancer cells and the myeloid cells, and
d) identifying targeted genes in the myeloid cells that enhance or inhibit myeloid cell-mediated killing of cancer cells.

14. A method of screening for a modulator of myeloid cell-mediated killing of cancer cells in the presence of a myeloid cell checkpoint inhibitor, comprising
a) providing a population of cancer cells expressing a targetable endonuclease and an sgRNA library targeting genes,
b) contacting the cancer cells with myeloid cells capable of having an anticancer response and the myeloid cell checkpoint inhibitor,
c) coculturing the cancer cells and myeloid cells with the myeloid cell checkpoint inhibitor, and
d) measuring the relative abundance of each sgRNA of the sgRNA library in the cocultured cancer cells as compared to the abundance of each gRNA in control cancer cells cocultured with myeloid cells but not the myeloid cell checkpoint inhibitor,
wherein the differential relative abundance of an sgRNA as compared to the control indicates that the gene targeted by the sgRNA is a candidate modulator of myeloid cell-mediated killing of cancer cells in the presence of the myeloid cell checkpoint inhibitor.

15. The method of claim 14, wherein the population of cancer cells of step a) has been transduced with a pool of sgRNA targeting every gene expressed on the cell surface in the genome of the cancer cell at a multiplicity of infection of about 0.2 to 0.4.

16. The method of claim 14, wherein the population of cancer cells of step a) has been transduced with a pool of sgRNA targeting every gene in the genome of the cancer cell at a multiplicity of infection of about 0.2 to 0.4.

17. The method of claims 14-16, wherein at least two sgRNA target each gene of the targeted genes.

18. The method of claims 14-17, wherein the cancer cells are a cancer cell line.

19. The method of claims 14-18, wherein the myeloid cells are macrophages.

20. The method of claims 14-19, wherein the population of cancer cells express the targetable endonuclease.

21. The method of claims 14-20, wherein the myeloid cell checkpoint inhibitor is a CD24 antibody, a CD47 antagonist, a CD40 agonist, or a PD-L1 antagonist.

22. The method of claims 14-21, wherein the cancer cells and myeloid cells are cocultured with the myeloid cell checkpoint inhibitor for 1 day or more in step c).

23. The method of claims 14-22, wherein an increased abundance of sgRNA targeting a gene as compared to the control indicates that the product of the gene enhances myeloid cell-mediated killing of cancer cells in the presence of a myeloid cell checkpoint inhibitor.

24. The method of claims 14-23, wherein a decreased abundance of sgRNA targeting a gene as compared to the control indicates that the product of the gene inhibits myeloid cell-mediated killing of cancer cells in the presence of a myeloid cell checkpoint inhibitor.

25. A method of screening for a modulator of myeloid cell-mediated killing of cancer cells in the presence of a myeloid cell checkpoint inhibitor, comprising
a) providing a population of cancer cells expressing a RNAi library,
b) contacting the cancer cells with myeloid cells capable of having an anticancer response and the myeloid cell checkpoint inhibitor,
c) coculturing the cancer cells and myeloid cells with the myeloid cell checkpoint inhibitor, and
d) measuring the relative abundance of each RNAi agent of the RNAi library in the cocultured cancer cells as compared to the abundance of each RNAi agent in control cancer cells cocultured with myeloid cells but not the myeloid cell checkpoint inhibitor,
wherein the differential relative abundance of an RNAi agent as compared to the control indicates that the gene targeted by the RNAi agent is a candidate modulator of myeloid cell-mediated killing of cancer cells in the presence of the myeloid cell checkpoint inhibitor.

26. A method of screening for a modulator of myeloid cell-mediated killing of cancer cells in the presence of a myeloid cell checkpoint inhibitor, comprising
a) providing a population of myeloid cells capable of having an anticancer response and expressing a targetable endonuclease and an sgRNA library targeting genes,
b) contacting cancer cells with the myeloid cells,
c) coculturing the cancer cells and myeloid cells with the myeloid cell checkpoint inhibitor, and
d) identifying targeted genes in the myeloid cells that enhance or inhibit myeloid cell-mediated killing of cancer cells.

27. A method of screening for a modulator of myeloid cell-mediated killing of cancer cells in the presence of a myeloid cell checkpoint inhibitor, comprising
a) providing a population of myeloid cells capable of having an anticancer response and expressing an RNAi library,
b) contacting cancer cells with the myeloid cells,
c) coculturing the cancer cells and myeloid cells with the myeloid cell checkpoint inhibitor, and
d) identifying targeted genes in the myeloid cells that enhance or inhibit myeloid cell-mediated killing of cancer cells.

28. A method of treating cancer in a subject comprising administering to the subject an agent that modulates the level or activity of a cancer cell gene that modulates macrophage-mediated cancer cell killing (MMCCK).

29. The method of claim 28, wherein the agent decreases the level or activity of the cancer cell gene and the cancer cell gene inhibits MMCCK.

30. The method of claim 28, wherein the cancer cell gene is selected from Met, Cd47, Igf1r, Arf1, Notch2, Afdn, Art1, Msn, Slc16a1, Gnai2, Sdc1, Cd4, Cd163, Cftr, Cd8a, Jam2, Icos, Nrg1, Ide, I112rb2, Has2, Gpc1, Insr, Epha2, Jmjd6, and Lrrc4.

31. The method of claims 28-30, wherein the agent is an antibody or functional fragment or derivative thereof to a cell surface receptor.

32. The method of claim 28, wherein the agent increases the level or activity of the cancer cell gene and the cancer cell gene enhances MMCCK.

33. The method of claim 28, wherein the cancer cell gene is selected from Acvr1b, Acvr2a, Adam9, Adcy1, Atp6ap2, Bmpr2, C5ar2, Cd320, Cd7, Cdc20, Cdh1, Cdh11, Epha4, Fxyd6, Gjb1, Hras, Ifn1r1, I110ra, I113ra1, 1121r, Itgav, Itgb1, Itgb3, Lamc2, Lrfn3, Plxnb2, Po1r1c, Psen1, Ptdss1, Pth2r, Ror2, Rtn4r12, Sor11, St14, Stx4a, Tfrc, T1r6, and Tspan1.

34. A method of treating cancer in a subject comprising administering to the subject a myeloid cell checkpoint inhibitor and an agent that modulates the level or activity of a cancer cell gene that modulates myeloid cell-mediated killing of cancer cells in the presence of the myeloid cell checkpoint inhibitor.

35. The method of claim 34, wherein the agent decreases the level or activity of the cancer cell gene and the cancer cell gene inhibits myeloid cell-mediated killing of cancer cells in the presence of the myeloid cell checkpoint inhibitor.

36. The method of claim 35, wherein the myeloid cell checkpoint inhibitor is an anti-CD24 binding antibody and the cancer cell gene is selected from Cd24a, Acvr1b, Acvr2a, Ncstn, Psen1, Itgb1, Tgfbr1, Epha2, Cd320, F2r, Nt5e, and Sdc1.

37. The method of claim 35, wherein the myeloid cell checkpoint inhibitor is an anti-CD47 blocking antibody and the cancer cell gene is selected from Rpsa, Acvr1b, Acvr2a, Ncstn, Alcam, Tmem222, Psen1, Igsf11, Fzd5, Plxnb2, Cadm1, and Lrp5.

38. The method of claim 35, wherein the myeloid cell checkpoint inhibitor is an anti-CD40 agonizing antibody and the cancer cell gene is selected from Rpsa, Cdc20, Mfrp, and Igf1r1.

39. The method of claim 35, wherein the myeloid cell checkpoint inhibitor is an anti-PD-L1 blocking antibody and the cancer cell gene is selected from Nectin2 and Ltk.

40. The method of claim 34, wherein the agent increases the level or activity of the cancer cell gene and the cancer cell gene enhances myeloid cell-mediated killing of cancer cells in the presence of the myeloid cell checkpoint inhibitor.

41. The method of claim 40, wherein the myeloid cell checkpoint inhibitor is an anti-CD24 antibody and the cancer cell gene is selected from Efnb3, Pdcd11g2, Hjv, Rnf43, Adam23, Havcr2, Lag3, Erbb2, Art1, Insr, T1r6, Cdh11, T1r2, I117rc, Adora2b, Tfrc, Dnajb11, Ramp3, Igf1r, Arf1, Acvr1, Afdn, Tnfsf13, Ld1r, Atp5b, Atp6ap2, Stx4a, Cdh1, and Cd47.

42. The method of claim 40, wherein the myeloid cell checkpoint inhibitor is an anti-CD47 blocking antibody and the cancer cell gene is selected from Itgb3, Cd99, Retn, Egfr, Atp6ap2, K1rb1a, Adam10, Lamp1, C5ar1, Sstr5, Lrfn3, Sema4b, Igf1r, Ld1r, Fam3c, Met, Erbb2, Cdh11, 1121r, I117rc, Adgrb2, Atp5b, Arf1, Copa, Acvr1, and Stx4a.

43. The method of claim 34, wherein the myeloid cell checkpoint inhibitor is an anti-CD40 activating antibody and the cancer cell gene is selected from I118r1, I127ra, Ephb2, Adam19, Pdcd1, and Copa.

44. The method of claim 34, wherein the myeloid cell checkpoint inhibitor is an anti-PD-L1 blocking antibody and the cancer cell gene is selected from Erbb3, Mp1, Ptprd, Mrc1, Tspan1, Egfr, I117rc, Sdc2, Stx3, Ntrk1, Sstr5, Cdh11, and Copa.

45. The method of claims 34-44, wherein the subject is a human or a mouse.

46. A pharmaceutical composition comprising a pharmaceutically acceptable excipient and an antibody or functional fragment or derivative thereof, small molecule, peptide or other agent specifically binding to Ermp1, Cflar, Slc35a1, Chst2, Copx, Map3k7, Efr3a, Dpm1, Dpm2, Dpm3, or PigP.

47. A pharmaceutical composition comprising a pharmaceutically acceptable excipient and an agent that increases the level or activity of Ptdss1, Mtf1, Zbtb14, or Pomp.
